(19) United States
(12) Patent Application Publication
(10) Pub. No.: US 2010/0222326 A1
(43) Pub. Date: Sep. 2, 2010

(54) New Heterocyclic Derivatives Useful For The Treatment of CNS Disorders

(75) Inventors: Benoit Kenda, Brussels (BE); Laurent Turet, Hamme-Mille (BE); Yannick Quesnel, Brussels (BE); Philippe Michel, Brussels (BE); Ali Ates, Brussels (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Brussels (BE)

(21) Appl. No.: 12/597,772

(22) PCT Filed: Apr. 24, 2008
(86) PCT No.: PCT/EP08/55022

§ 371 Date: May 4, 2010

(30) Foreign Application Priority Data
Apr. 27, 2007 (EP) EP 07008595.6

(51) Int. Cl. A61K 31/55; A61K 31/4164; A61K 31/421; A61K 31/40; A61K 31/426; A61K 31/5025; A61K 31/454; A61K 31/437; A61K 31/4178; A61K 31/4725; A61K 31/4709; A61P 25/00; A61P 25/16; A61P 25/24; A61P 25/22; A61P 25/06; A61P 9/00; A61P 9/10; A61P 35/00; C07D 233/32; C07D 403/06; C07D 471/04; C07D 487/04; C07D 263/34; C07D 207/36; C07D 211/76; C07D 223/10; C07D 223/14; C07D 215/227; C07D 495/04

(52) U.S. Cl. 514/212.07; 514/386; 514/376; 514/424; 514/369; 514/248; 514/326; 514/300; 514/212.08; 514/397; 514/309; 514/312; 548/324.1; 548/312.7; 546/121; 544/236; 548/187; 548/229; 548/543; 546/243; 540/485; 540/523; 546/158; 546/139; 548/453

(57) ABSTRACT

The present invention relates to new compounds, processes for preparing them, pharmaceutical compositions containing them and their use as pharmaceuticals.

### INTRODUCTION

The present invention relates to heterocyclic compounds, processes for preparing them, pharmaceutical compositions containing them and their use as pharmaceuticals.

European Patent No. 0 162 036 B1 discloses compound (S)-α-ethyl-2-oxo-1-pyrrolidine acetamide, which is known under the International Nonproprietary Name (INN) Levetiracetam.

Levetiracetam, a laevorotary compound, is disclosed as a protective agent for the treatment and prevention of hypoxic and ischemic type aggressions of the central nervous system. This compound is also effective in the treatment of epilepsy, a therapeutic indication for which it has been demonstrated that its dextrorotatory enantiomer (R)-α-ethyl-2-oxo-1-pyrrolidine acetamide, also known from European Patent No. 0 165 919 B1, completely lacks activity (Gower A. J. et al., Eur. J. Pharmacol. (1992), 222, 193-203).

Belavin I. Yu. et al. (Khimiko-Farmatsevticheskii Zhurnal (1992), 26 (9-10), 74-76) discloses 1-[1-(1H-benzimidazol-1-yl)ethyl]-2-pyrrolidinone and its anticonvulsant activity.

WO 01/62726 discloses pyrrolidinone compounds having the following formula:

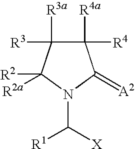

WO 2005/054188 discloses imidazole derivatives having the following formula:

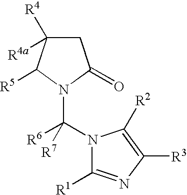

The imidazole or benzimidazole is attached by a nitrogen to the methylene linker of the pyrrolidinone.

WO 02/094787 discloses.compounds having the formula:

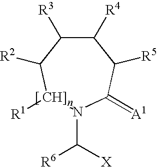

WO 2005/118561 discloses benzoxazolone compounds of the formula:

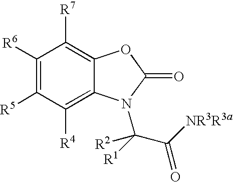

WO 2006/128692 discloses compounds of the formula:

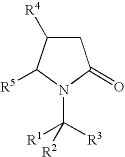

GB-1,036,280 discloses imidazole derivatives.

U.S. Pat. No. 4,650,796 discloses 3-acylaminomethylimidazo[1,2-a]pyridine derivatives.

FR-2,320,742 as well as Pinza et al in II Farmaco-Ed Sc.-vol. 33-fasc 2, 1977 disclose pyrrolidine acetamides.

BE-857,191 discloses 1,2,4,5-tetrahydro-3H-2-benzazepine-3-ones.

It has now surprisingly been found that certain heterocyclic derivatives are useful in the treatment of various CNS disorders including epilepsy.

### SUMMARY OF THE INVENTION

The invention provides compounds having the formula (I) their geometrical isomers, enantiomers, diastereoisomers and mixtures, or a pharmaceutically acceptable salt thereof,

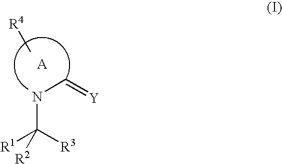

in particular for the manufacture of a medicament for the treatment or prevention of epilepsy, epileptogenesis, seizure disorders, convulsions, Parkinson's disease, dyskinesia induced by dopamine replacement therapy, tardive dyskinesia induced by administration of neuroleptic drugs, Huntington Chorea, and other neurological disorders including bipolar disorders, mania, depression, anxiety, panic disorders, attention deficit hyperactivity disorder (ADHD), migraine, trigeminal and other neuralgia, chronic pain, neuropathic pain, cerebral ischemia, cardiac arrhythmia, myotonia, cocaine abuse, stroke, myoclonus, tremor, essential tremor, simple or complex tics, Tourette syndrome, restless leg syndrome and other movement disorders, neonatal cerebral haemorrhage, amyotrophic lateral sclerosis, spasticity and degenerative diseases, subjective tinnitus, apathy syndrome, bronchial asthma, asthmatic status and allergic bronchitis, asthmatic syndrome, bronchial hyperreactivity and bronchospastic syndromes, lower urinary tract disorders, as well as allergic and vasomotor rhinitis and rhinoconjunctivitis.

Further aspects of the invention will become apparent from the detailed description.

A first aspect of the invention consists in compounds having the formula (I), their geometrical isomers, enantiomers, diastereomers and mixtures, or a pharmaceutically acceptable salt thereof, for the treatment or prevention of epilepsy, epileptogenesis, seizure disorders, convulsions, Parkinson's disease, dyskinesia induced by dopamine replacement therapy, tardive dyskinesia induced by administration of neuroleptic drugs, Huntington Chorea, and other neurological disorders including bipolar disorders, mania, depression, anxiety, panic disorders, attention deficit hyperactivity disorder (ADHD), migraine, trigeminal and other neuralgia, chronic pain, neuropathic pain, cerebral ischemia, cardiac arrhythmia, myotonia, cocaine abuse, stroke, myoclonus, tremor, essential tremor, simple or complex tics, Tourette syndrome, restless leg syndrome and other movement disorders, neonatal cerebral haemorrhage, amyotrophic lateral sclerosis, spasticity and degenerative diseases, subjective tinnitus, apathy syndrome, bronchial asthma, asthmatic status and allergic bronchitis, asthmatic syndrome, bronchial hyperreactivity and bronchospastic syndromes, lower urinary tract disorders, as well as allergic and vasomotor rhinitis and rhinoconjunctivitis.

Compounds of formula (I) are as follows:

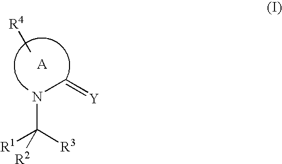

wherein

Y is O, S or NR⁸;

R¹ is hydrogen or C₁₋₆ alkyl;

R² is hydrogen;

R³ is —CONR⁵R⁶, —COR⁷, an imidazolyl, an imidazopyridinyl, an imidazopyridazinyl or a 1H-indol-1-yl;

R⁵, R⁶ are the same or different and are independently selected from hydrogen and C₁₋₆ alkyl;

R⁷ is C₁₋₆ alkyl;

A is a monocyclic or bicyclic heterocyclic moiety selected from the group consisting of imidazolidin-1-yl, 1,3-oxazolidin-3-yl, 2,5-dihydro-1H-pyrrol-1-yl, 1,3-thiazol-3(2H)-yl, 1,3-thiazolidin-3-yl, pyrrolidin-1-yl, piperidin-1-yl, azepan-1-yl, 5,6-dihydro-4H-thieno[3,2-b]pyrrol-4-yl, hexahydro-4H-thieno[3,2-b]pyrrol-4-yl, 2,3-dihydro-1H-thieno[3,4-b]pyrrol-1-yl, 1,3-benzothiazol-3(2H)-yl, 1,3-benzoxazol-3(2H)-yl, pyrazolo[1,5-a]pyridin-1(2H)-yl, 3,4-dihydroisoquinolin-2(1H)-yl, 3,4-dihydroquinolin-1(2H)-yl, 1,3,4,5-tetrahydro-2H-2-benzazepin-2-yl, 1,2,4,5-tetrahydro-3H-3-benzazepin-3-yl;

R⁴ is either R^{4a} or R^{4b} depending on whether A being is a monocyclic or a bicyclic heterocycle:
where A is a monocyclic heterocyclic moiety, R⁴ is R^{4a} which is selected from the group consisting of hydrogen; C₁₋₆ alkyl optionally substituted by a substituent selected from halogen, C₁₋₄ alkoxy, C₁₋₄ alkylthio, azido, nitrooxy or an aryl; C₂₋₆ alkenyl optionally substituted by halogen; C₂₋₆ alkynyl optionally substituted by halogen; azido; alkoxycarbonylamino; arylsulfonyloxy; a substituted or unsubstituted aryl; or a 3-8 membered substituted or unsubstituted heterocycle;
where A is a bicyclic heterocyclic moiety R⁴ is R^{4b} which is selected from the group comprising or consisting of hydrogen; nitro; cyano; halogen; heterocycle; amino; aryl; C₁₋₆ alkyl optionally substituted by at least one halogen; or C₁₋₆ alkoxy optionally substituted by at least one halogen;

R⁸ is cyano (CN) or C₁₋₆ alkylsulfonyl(—SO₂-alkyl);

with the proviso that:
For compounds where A is selected from a piperidin-1-yl, azepan-1-yl, a 1,3-benzothiazol-3(2H)-yl or a 1,3-benzoxazol-3(2H)-yl and Y is O or S, R³ must be selected from an imidazolyl, an imidazopyridinyl, an imidazopyridazinyl or a 1H-indol-1-yl.
For compounds where A is a imidazolidin-1-yl, Y is O, R¹ and R² are hydrogen, R³ is —CONR⁵R⁶, R⁵ and R⁶ are as above defined, then R^{4a} may not be an alkyl, aralkyl or substituted aralkyl.
Where A is either of a piperidin-1-yl and a azepan-1-yl and Y is O, R¹, R² and R^{4a} are all hydrogen, then R³ could not be a 2-phenylimidazo[1,2-a]pyridin-3-yl.
where A is pyrrolidin-1-yl, Y is NR⁸.
where A is pyrrolidin-1-yl, piperidin-1-yl or a azepan-1-yl, R³ is —CONR⁵R⁶ or —COR⁷, Y is NR⁸ and R⁸ is CN, then R^{4a} is different from hydrogen.

In a specific embodiment, compounds of formula (I) are as follows:

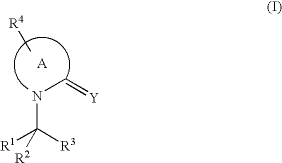

wherein

Y is O or S; preferably Y is O.

R¹ is hydrogen or C₁₋₆ alkyl;

R² is hydrogen;

R³ is —CONR⁵R⁶, —COR⁷, an imidazolyl, an imidazopyridinyl, an imidazopyridazinyl;

R⁵, R⁶ are the same or different and are independently selected from hydrogen and C₁₋₆ alkyl;

R⁷ is C₁₋₆ alkyl;

A is a monocyclic or bicyclic heterocyclic moiety selected from the group consisting of imidazolidin-1-yl, 1,3-oxazolidin-3-yl, 2,5-dihydro-1H-pyrrol-1-yl, 1,3-thiazol-3(2H)-yl, 1,3-thiazolidin-3-yl, piperidin-1-yl, azepan-1-yl, 5,6-dihydro-4H-thieno[3,2-b]pyrrol-4-yl, hexahydro-4H-thieno[3,2-b]pyrrol-4-yl, 2,3-dihydro-1H-thieno[3,4-b]pyrrol-1-yl, 1,3-benzothiazol-3(2H)-yl, 1,3-benzoxazol-3(2H)-yl, pyrazolo[1,5-a]pyridin-1(2H)-yl, 3,4-dihydroisoquinolin-2(1H)-yl, 3,4-dihydroquinolin-1(2H)-yl, 1,3,4,5-tetrahydro-2H-2-benzazepin-2-yl, 1,2,4,5-tetrahydro-3H-3-benzazepin-3-yl;

R⁴ is either R^{4a} or R^{4b} depending on whether A being is a monocyclic or a bicyclic heterocycle:
where A is a monocyclic heterocyclic moiety, R⁴ is R^{4a} which is selected from the group consisting of hydrogen; C₁₋₆ alkyl optionally substituted by a substituent selected from halogen, C₁₋₄ alkoxy, C₁₋₄ alkylthio, azido, nitrooxy or an aryl; C₂₋₆ alkenyl optionally substituted by halogen; C₂₋₆ alkynyl optionally substituted by halogen; azido; alkoxycarbonylamino; arylsulfonyloxy; a substituted or unsubstituted aryl; or a 3-8 membered substituted or unsubstituted heterocycle;
where A is a bicyclic heterocyclic moiety R⁴ is R^{4b} which is selected from the group comprising or consisting of hydrogen; nitro; cyano; halogen; heterocycle; amino; aryl; C₁₋₆ alkyl optionally substituted by at least one halogen; or C₁₋₆ alkoxy optionally substituted by at least one halogen;

with the proviso that:
For compounds where A=Y is selected from a 2-oxo-piperidin-1-yl, a 2-oxo-azepan-1-yl, a 2-oxo-1,3-benzothiazol-3(2H)-yl or a 2-oxo-1,3-benzoxazol-3(2H)-yl, R³ must be selected from an imidazolyl, an imidazopyridinyl or an imidazopyridazinyl.
For compounds where A=Y is a 5-oxoimidazolidin-1-yl, R¹ and R² are hydrogen, R³ is —CONR⁵R⁶, R⁵ and R⁶ are as above defined, then R⁴a may not be an alkyl, aralkyl or substituted aralkyl.
Where A=Y is either of a 2-oxo-piperidin-1-yl and a 2-oxo-azepan-1-yl, R¹, R² and R^{4a} are all hydrogen, then R³ could not be a 2-phenylimidazo[1,2-a]pyridin-3-yl.

In another specific embodiment, compounds of formula (I) are as follows:

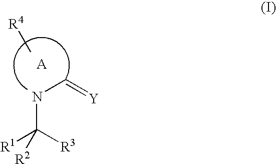

wherein

Y is NR⁸;

R¹ is hydrogen or C₁₋₆ alkyl;

R² is hydrogen;

R³ is —CONR⁵R⁶, —COR⁷, an imidazolyl, an imidazopyridinyl, an imidazopyridazinyl or an 1H-indol-1-yl;

R⁵, R⁶ are the same or different and are independently selected from hydrogen and C₁₋₆ alkyl;

R⁷ is C₁₋₆ alkyl;

A is a monocyclic or bicyclic heterocyclic moiety selected from the group consisting of imidazolidin-1-yl, 1,3-oxazolidin-3-yl, 2,5-dihydro-1H-pyrrol-1-yl, 1,3-thiazol-3(2H)-yl, 1,3-thiazolidin-3-yl, pyrrolidin-1-yl, piperidin-1-yl, azepan-1-yl, 5,6-dihydro-4H-thieno[3,2-b]pyrrol-4-yl, hexahydro-4H-thieno[3,2-b]pyrrol-4-yl, 2,3-dihydro-1H-thieno[3,4-b]pyrrol-1-yl, 1,3-benzothiazol-3(2H)-yl, 1,3-benzoxazol-3(2H)-yl, pyrazolo[1,5-a]pyridin-1(2H)-yl, 3,4-dihydroisoquinolin-2(1H)-yl, 3,4-dihydroquinolin-1(2H)-yl, 1,3,4,5-tetrahydro-2H-2-benzazepin-2-yl, 1,2,4,5-tetrahydro-3H-3-benzazepin-3-yl;

R⁴ is either R^{4a} or R^{4b} depending on whether A being is a monocyclic or a bicyclic heterocycle:
where A is a monocyclic heterocyclic moiety, R⁴ is R^{4a} which is selected from the group consisting of hydrogen; C₁₋₆ alkyl optionally substituted by a substituent selected from halogen, C₁₋₄ alkoxy, C₁₋₄ alkylthio, azido, nitrooxy or an aryl; C₂₋₆ alkenyl optionally substituted by halogen; C₂₋₆ alkynyl optionally substituted by halogen; azido; alkoxycarbonylamino; arylsulfonyloxy; a substituted or unsubstituted aryl; or a 3-8 membered substituted or unsubstituted heterocycle;
where A is a bicyclic heterocyclic moiety R⁴ is R^{4b} which is selected from the group comprising or consisting of hydrogen; nitro; cyano; halogen; heterocycle; amino; aryl; C₁₋₆ alkyl optionally substituted by at least one halogen; or C₁₋₆ alkoxy optionally substituted by at least one halogen;

R⁸ is cyano (CN) or C₁₋₆ alkylsulfonyl(—SO₂-alkyl);

with the proviso that where A is pyrrolidin-1-yl, piperidin-1-yl or a azepan-1-yl, R³ is —CONR⁵R⁶ or —COR⁷, Y is NR⁸ and R⁸ is CN, then R^{4a} is different from hydrogen.

In another specific embodiment, compounds of formula (I) are as follows:

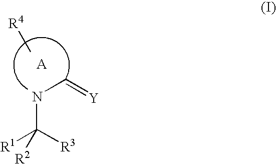

wherein

Y is O, S or NR⁸;

R¹ is hydrogen or C₁₋₆ alkyl;

R² is hydrogen;

R³ is an 1H-indol-1-yl:

A is a monocyclic or bicyclic heterocyclic moiety selected from the group consisting of imidazolidin-1-yl, 1,3-oxazolidin-3-yl, 2,5-dihydro-1H-pyrrol-1-yl, 1,3-thiazol-3(2H)-yl, 1,3-thiazolidin-3-yl, pyrrolidin-1-yl, piperidin-1-yl, azepan-1-yl, 5,6-dihydro-4H-thieno[3,2-b]pyrrol-4-yl, hexahydro-4H-thieno[3,2-b]pyrrol-4-yl, 2,3-dihydro-1H-thieno[3,4-b]pyrrol-1-yl, 1,3-benzothiazol-3(2H)-yl, 1,3-benzoxazol-3(2H)-yl, pyrazolo[1,5-a]pyridin-1(2H)-yl, 3,4-dihydroisoquinolin-2(1H)-yl, 3,4-dihydroquinolin-1(2H)-yl, 1,3,4,5-tetrahydro-2H-2-benzazepin-2-yl, 1,2,4,5-tetrahydro-3H-3-benzazepin-3-yl;

R⁴ is either R^{4a} or R^{4b} depending on whether A being is a monocyclic or a bicyclic heterocycle:
where A is a monocyclic heterocyclic moiety, R⁴ is R^{4a} which is selected from the group consisting of hydrogen; C₁₋₆ alkyl optionally substituted by a substituent selected from halogen, C₁₋₄ alkoxy, C₁₋₄ alkylthio, azido, nitrooxy or an aryl; C₂₋₆ alkenyl optionally substituted by halogen; C₂₋₆ alkynyl optionally substituted by halogen; azido; alkoxycarbonylamino; arylsulfonyloxy; a substituted or unsubstituted aryl; or a 3-8 membered substituted or unsubstituted heterocycle;
where A is a bicyclic heterocyclic moiety R⁴ is R^{4b} which is selected from the group comprising or consisting of hydrogen; nitro; cyano; halogen; heterocycle; amino; aryl; C₁₋₆ alkyl optionally substituted by at least one halogen; or C₁₋₆ alkoxy optionally substituted by at least one halogen;

R⁸ is cyano (CN) or C₁₋₆ alkylsulfonyl(—SO₂-alkyl);

In a specific embodiment the A=Y group is selected from the list consisting of:

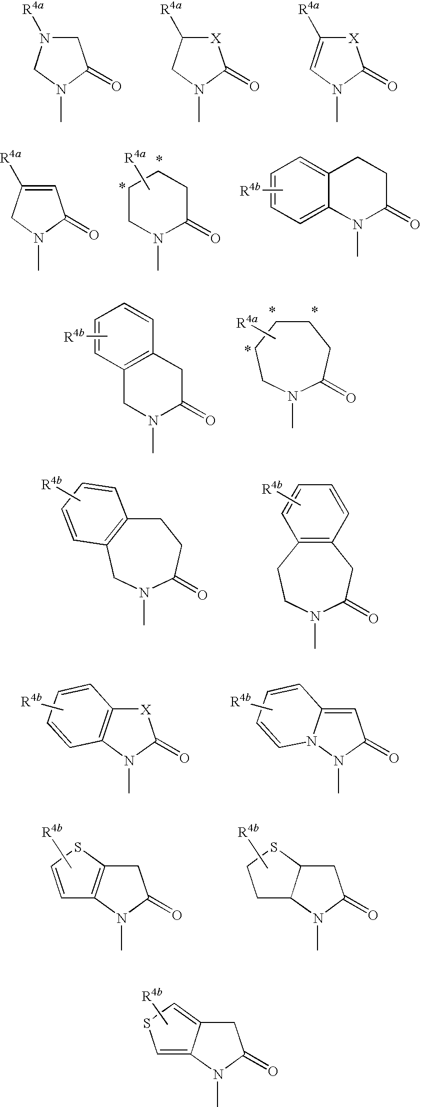

wherein X is O or S, in a more specific embodiment O; in another embodiment, X is S.

The asterisks in the above illustration indicate the attachment sites of the substituent R^{4a}.

In another specific embodiment the A=Y group is selected from the list consisting of:

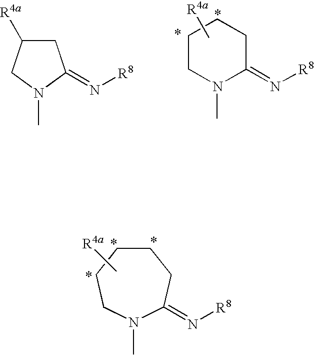

wherein R⁸ is cyano (CN) or C₁₋₆ alkylsulfonyl.

The asterisks in the above illustration indicate the attachment sites of the substituent R^{4a}.

The compounds of the present invention are particularly useful for the treatment of epilepsy.

In a specific embodiment, Y is O.

In a further specific embodiment, Y is NR⁸.

In a specific embodiment, when R³ is —CONR⁵R⁶ and R¹ is C₁₋₆ alkyl, the carbon atom to which R¹ and R³ are attached is preferably in the “S”-configuration.

In a specific embodiment R¹ is hydrogen, methyl, ethyl and R² is hydrogen.

In a specific embodiment R³ is —CONH₂.

In a more specific embodiment, R³ is —CONH₂ and the carbon atom to which R³ is attached is in the “S”-configuration.

In a further specific embodiment R³ is 1H-imidazol-1-yl, 1H-imidazol-4-yl, 1H-imidazol-5-yl, imidazo[1,2-a]pyridin-3-yl or imidazo[1,2-b]pyridazin-3-yl.

In another specific embodiment R³ is 1H-indol-1-yl.

In a specific embodiment R^{4a} is a C₁₋₆ alkyl which may optionally be substituted by a halogen; or a phenyl.

In another specific embodiment R^{4b} is hydrogen, halogen, nitro, cyano or a C₁₋₆ alkyl optionally substituted by a halogen.

In still a further embodiment compounds may be used in the treatment of the above mentioned disorders, in particular of epilepsy, having the formula (I-E), as wells as its geometrical isomers, enantiomers, diastereomers and mixtures, or a pharmaceutically acceptable salt thereof,

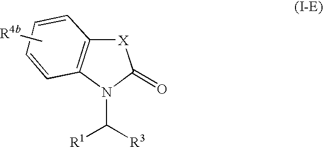

wherein

X is O or S;

R¹ is hydrogen or C₁₋₆ alkyl, in a more specific embodiment hydrogen;

R³ is an imidazolyl, an imidazopyridinyl, an imidazopyridazinyl;

R^{4b} is hydrogen; nitro; cyano; halogen; C₁₋₆ alkyl optionally substituted by halogen; C₁₋₆ alkoxy optionally substituted by halogen.

A further aspect of the present invention consists in novel compounds having the formula (I-A), their geometrical isomers, enantiomers, diastereomers and mixtures, or a pharmaceutically acceptable salt thereof,

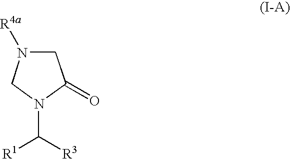

wherein

R¹ is hydrogen or C₁₋₆ alkyl, preferably hydrogen, methyl or ethyl; in a more specific embodiment R¹ is ethyl.

R³ is —CONH₂, an imidazolyl, an imidazopyridinyl, an imidazopyridazinyl, preferably R³ is —CONH₂.

R^{4a} is either hydrogen or an aryl; with the proviso that 2-(5-oxoimidazolidin-1-yl)acetamide is excluded. Preferably R^{4a} is an aryl, e.g. a phenyl which may be substituted preferably by halogen, nitro, alkoxy, in particular by nitro.

In a particular embodiment, when R³ is —CONH₂ and R¹ is C₁₋₆ alkyl, the carbon atom to which R¹ and R³ are attached is preferably in the “S”-configuration.

A further aspect of the present invention consists in novel compounds having the formula (I-B1 or I-B2), their geometrical isomers, enantiomers, diastereomers and mixtures, or a pharmaceutically acceptable salt thereof,

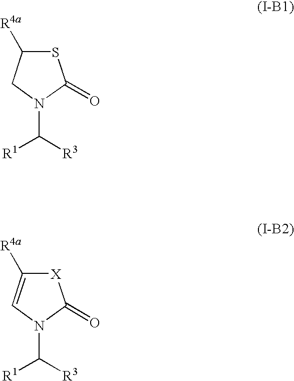

wherein X in formula (I-B2) is either S or O, in a more specific embodiment S;

R¹ is hydrogen or C₁₋₆ alkyl, preferably hydrogen, methyl or ethyl; in a more specific embodiment R¹ is ethyl.

R³ is —CONH₂, an imidazolyl, an imidazopyridinyl, an imidazopyridazinyl; preferably R³ is —CONH₂.

R^{4a} is hydrogen; C₁₋₆ alkyl optionally substituted by halogen or C₁₋₄ alkoxy; an aryl; or C₂₋₆ alkenyl optionally substituted by halogen. Preferably, R^{4a} is C₁₋₆ alkyl optionally substituted by halogen or C₂₋₆ alkenyl optionally substituted by halogen or an aryl. In a more specific embodiment R^{4a} is C₁₋₆ alkyl optionally substituted by halogen or aryl.

In a particular embodiment, when R³ is —CONH₂ and R¹ is C₁₋₆ alkyl, the carbon atom to which R¹ and R³ are attached is preferably in the “S”-configuration.

A further aspect of the present invention consists in novel compounds having the formula (I-B3), their geometrical isomers, enantiomers, diastereomers and mixtures, or a pharmaceutically acceptable salt thereof,

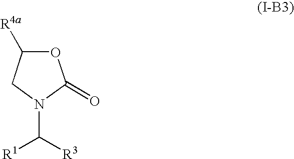

wherein

R¹ is either hydrogen or C₁₋₆ alkyl, preferably hydrogen, methyl or ethyl; more preferably R¹ is ethyl.

R³ is —CONH₂, an imidazolyl, an imidazopyridinyl, an imidazopyridazinyl; preferably R³ is —CONH₂.

R^{4a} is C₁₋₆ alkyl optionally substituted by halogen or C₁₋₄ alkoxy; an aryl; or C₂₋₆ alkenyl optionally substituted by halogen. Preferably, R^{4a} is C₁₋₆ alkyl optionally substituted by halogen or C₂₋₆ alkenyl optionally substituted by halogen.

In a particular embodiment, when R³ is —CONH₂ and R¹ is C₁₋₆ alkyl, the carbon atom to which R¹ and R³ are attached is preferably in the “S”-configuration.

A further aspect of the present invention consists in novel compounds having the formula (I-C), their geometrical isomers, enantiomers, diastereomers and mixtures, or a pharmaceutically acceptable salt thereof,

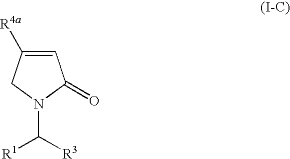

wherein

R¹ is hydrogen or C₁₋₆ alkyl, in particular hydrogen, methyl or ethyl.

R³ is —CONH₂, an imidazolyl, an imidazopyridinyl, an imidazopyridazinyl; in particular R³ is —CONH₂

R^{4a} is methyl, ethyl, butyl optionally substituted by halogen or C₁₋₄ alkoxy, an unsubstituted phenyl or a phenyl substituted by halogen, a C₁₋₆ alkyl optionally substituted by halogen or a C₁₋₄ alkoxy; or R^{4a} is a C₂₋₆ alkenyl optionally substituted by halogen. Preferably, R^{4a} is methyl, optionally substituted by halogen, an unsubstituted phenyl or a phenyl substituted by halogen.

In a particular embodiment, when R³ is —CONH₂ and R¹ is C₁₋₆ alkyl, the carbon atom to which R¹ and R³ are attached is preferably in the “S”-configuration.

A further aspect of the present invention consists in compounds having the formula (I-D1 or I-D2), their geometrical isomers, enantiomers, diastereomers and mixtures, or a pharmaceutically acceptable salt thereof,

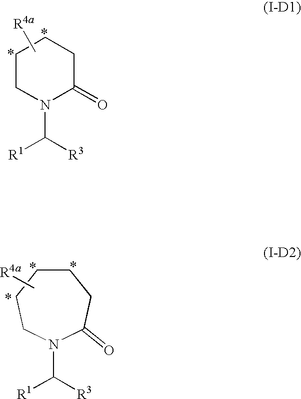

wherein

R¹ is hydrogen or C₁₋₆ alkyl, in particular hydrogen;

R³ is an imidazolyl, an imidazopyridinyl, an imidazopyridazinyl or a 1H-indol-1-yl. In one embodiment, R³ is 1H-imidazol-1-yl, 1H-imidazol-4-yl, 1H-imidazol-5-yl, imidazo[1,2-a]pyridin-3-yl, imidazo[1,2-b]pyridazin-3-yl or 1H-indol-1-yl. In a more specific embodiment, R³ is 1H-imidazol-1-yl, 1H-imidazol-4-yl, 1H-imidazol-5-yl, imidazo[1,2-a]pyridin-3-yl or 1H-indol-1-yl;

R^{4a} is hydrogen, C₁₋₆ alkyl optionally substituted by halogen or C₁₋₄ alkoxy; aryl; or C₂₋₆ alkenyl optionally substituted by halogen. In a specific embodiment, R^{4a} is C₁₋₆ alkyl optionally substituted by halogen; aryl; or C₂₋₆ alkenyl optionally substituted by halogen. In a more specific embodiment R^{4a} is C₁₋₆ alkyl optionally substituted by halogen; or aryl; e.g, propyl or phenyl;

with the proviso that when R¹ and R^{4a} are hydrogen, R³ is not 2-phenylimidazo[1,2-a]pyridin-3-yl.

A further aspect of the present invention consists in compounds having the formula (I-F1, I-F2 or I-F3), their geometrical isomers, enantiomers, diastereomers and mixtures, or a pharmaceutically acceptable salt thereof,

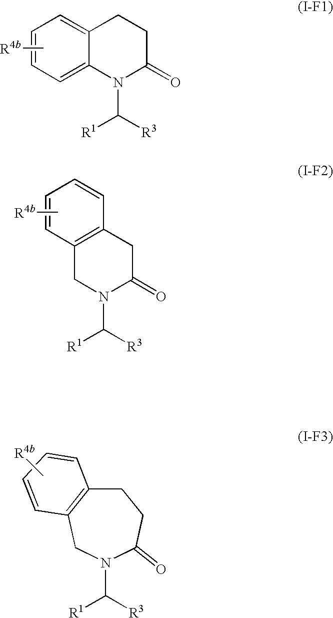

wherein

R¹ is hydrogen or C₁₋₆ alkyl, preferably hydrogen, methyl or ethyl; more preferably, R¹ is hydrogen.

R³ is —CONH₂, an imidazolyl, an imidazopyridinyl or an imidazopyridazinyl; in a more specific embodiment R³ is —CONH₂, 1H-imidazol-1-yl, 1H-imidazol-4-yl, 1H-imidazol-5-yl, imidazo[1,2-a]pyridin-3-yl or imidazo[1,2-b]pyridazin-3-yl.

R^{4b} is hydrogen; halogen; nitro; cyano; C₁₋₄ alkyl optionally substituted by halogen; C₁₋₄ alkoxy optionally substituted by halogen. In a more specific embodiment R^{4b} is hydrogen, halogen or cyano, more specifically halogen.

In a particular embodiment, when R³ is —CONH₂ and R¹ is C₁₋₆ alkyl, the carbon atom to which R¹ and R³ are attached is preferably in the “S”-configuration.

A further aspect of the present invention consists in compounds having the formula (I-F4), their geometrical isomers, enantiomers, diastereomers and mixtures, or a pharmaceutically acceptable salt thereof,

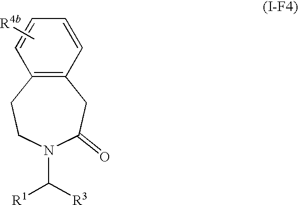

wherein

R¹ is hydrogen or C₁₋₆ alkyl, preferably hydrogen;

R³ is an imidazolyl, an imidazopyridinyl or an imidazopyridazinyl; more specifically R³ is 1H-imidazol-1-yl, 1H-imidazol-4-yl, 1H-imidazol-5-yl, imidazo[1,2-a]pyridin-3-yl or imidazo[1,2-b]pyridazin-3-yl. More specifically R³ is 1H-imidazol-4-yl or imidazo[1,2-a]pyridin-3-yl.

R^{4b} is hydrogen; halogen; nitro; cyano; C₁₋₄ alkyl optionally substituted by halogen; C₁₋₄ alkoxy optionally substituted by halogen; specifically R^{4b} is hydrogen, halogen or cyano.

In a particular embodiment, when R³ is —CONH₂ and R¹ is C₁₋₆ alkyl, the carbon atom to which R¹ and R³ are attached is preferably in the “S”-configuration.

A further aspect of the present invention consists in compounds having either of the formula (I-G1, I-G2 or I-G3), their geometrical isomers, enantiomers, diastereomers and mixtures, or a pharmaceutically acceptable salt thereof,

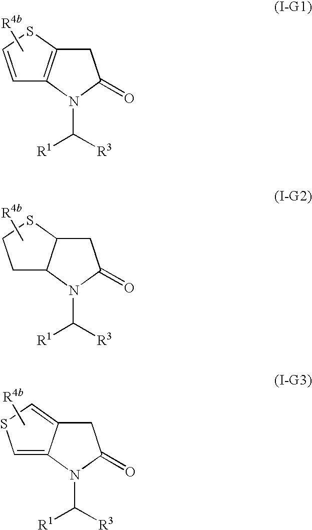

wherein

R¹ is hydrogen or C₁₋₆ alkyl; preferably hydrogen;

R³ is —CONH₂, an imidazolyl, an imidazopyridinyl, an imidazopyridazinyl; in a more specific embodiment R³ is —CONH₂, 1H-imidazol-1-yl, 1H-imidazol-4-yl, 1H-imidazol-5-yl, imidazo[1,2-a]pyridin-3-yl or imidazo[1,2-b]pyridazin-3-yl. In a even more specific embodiment R³ is an 1H-imidazol-4-yl or imidazo[1,2-a]pyridin-3-yl;

R^{4b} is hydrogen; halogen; C₁₋₄ alkyl optionally substituted by halogen; C₁₋₄ alkoxy optionally substituted by halogen.

In a particular embodiment, when R³ is —CONH₂ and R¹ is C₁₋₆ alkyl, the carbon atom to which R¹ and R³ are attached is preferably in the “S”-configuration.

A further aspect of the present invention consists in compounds having either of the formula (I-H1, I-H2 or I-H3), their geometrical isomers, enantiomers, diastereomers and mixtures, or a pharmaceutically acceptable salt thereof,

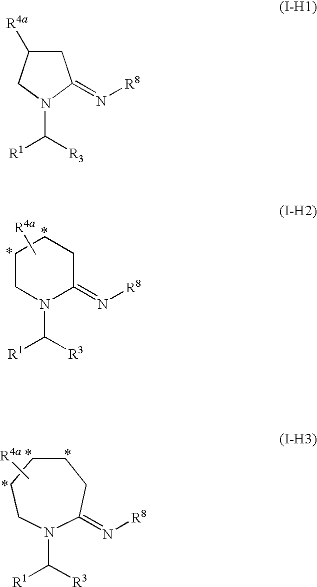

wherein

R¹ is hydrogen or C₁₋₆ alkyl; preferably hydrogen or methyl or ethyl;

R³ is —CONH₂ or an imidazolyl; preferably —CONH₂;

R⁸ is cyano or C₁₋₆ alkylsulfonyl;

R^{4a} is hydrogen, C₁₋₆ alkyl optionally substituted by halogen or C₁₋₄ alkoxy; aryl; or C₂₋₆ alkenyl optionally substituted by halogen. In a specific embodiment, R^{4a} is C₁₋₆ alkyl optionally substituted by halogen; aryl; or C₂₋₆ alkenyl optionally substituted by halogen. In a more specific embodiment R^{4a} is C₁₋₆ alkyl optionally substituted by halogen; or aryl; e.g, propyl;

with the proviso that where R⁸ is CN and R³ is —CONH2, then R^{4a} is not hydrogen.

In a particular embodiment, when R³ is —CONH₂ and R¹ is C₁₋₆ alkyl, the carbon atom to which R¹ and R³ are attached is preferably in the “S”-configuration.

Specific compounds of the present invention are those selected from the group consisting of: (2S)-2-[3-(4-nitrophenyl)-5-oxoimidazolidin-1-yl]butanamide; (2S)-2-[3-(2,4-dinitrophenyl)-5-oxoimidazolidin-1-yl]butanamide; (2S)-2-(5-oxo-3-phenylimidazolidin-1-yl)butanamide; 2[5-(iodomethyl)-2-oxo-1,3-oxazolidin-3-yl]butanamide; 2-(2-oxo-2,5-dihydro-1H-pyrrol-1-yl)butanamide; 2-(2-oxo-4-phenyl-2,5-dihydro-1H-pyrrol-1-yl)butanamide; 2-(4-methyl-2-oxo-2,5-dihydro-1H-pyrrol-1-yl)butanamide; (2S)-2-(2-oxo-5-propyl-1,3-thiazol-3(2H)-yl)butanamide; 2-(2-oxo-5-propyl-1,3-thiazol-3(2H)-yl)propanamide; 2-(5-butyl-2-oxo-1,3-thiazolidin-3-yl)butanamide; 2-(5-butyl-2-oxo-1,3-thiazolidin-3-yl)propanamide; 2-(2-oxo-5-phenyl-1,3-thiazolidin-3-yl)propanamide; 2-(2-oxo-5-propyl-1,3-thiazolidin-3-yl)butanamide; 2-(2-oxo-5-phenyl-1,3-thiazolidin-3-yl)butanamide; 2-(2-oxo-5-propyl-1,3-thiazolidin-3-yl)propanamide; (2S)-2-[2-oxo-5-(2,2,2-trifluoroethyl)-1,3-thiazolidin-3-yl]butanamide; 1-{[6-chloro-2-(trifluoromethyl)imidazo[1,2-b]pyridazin-3-yl]methyl}piperidin-2-one; 1-(1H-imidazol-4-ylmethyl)-5-propylpiperidin-2-one; 1-(1H-imidazol-1-ylmethyl)-5-propylpiperidin-2-one; 1-(imidazo[1,2-a]pyridin-3-ylmethyl)-5-propylpiperidin-2-one; 1-(1H-imidazol-1-ylmethyl)-5-phenylpiperidin-2-one; 1-(imidazo[1,2-a]pyridin-3-ylmethyl)-5-phenylpiperidin-2-one; 1-(imidazo[1,2-a]pyridin-3-ylmethyl)-4-phenylpiperidin-2-one; 1-(1H-imidazol-1-ylmethyl)-4-phenylpiperidin-2-one; 1-(imidazo[1,2-a]pyridin-3-ylmethyl)-4-propylpiperidin-2-one; 1-(1H-imidazol-5-ylmethyl)-4-propylpiperidin-2-one; 1-(1H-imidazol-1-ylmethyl)-4-propylpiperidin-2-one; 1-{[6-chloro-2-(trifluoromethyl)imidazo[1,2-b]pyridazin-3-yl]methyl}azepan-2-one; 1-(1H-imidazol-5-ylmethyl)-5-propylazepan-2-one; 5-propyl-1-{[2-(trifluoromethyl)imidazo[1,2-a]pyridin-3-yl]methyl}azepan-2-one; 5-phenyl-1-{[2-(trifluoromethyl)imidazo[1,2-a]pyridin-3-yl]methyl}azepan-2-one; 1-(1H-imidazol-5-ylmethyl)-6-propylazepan-2-one; 1-(1H-imidazol-4-ylmethyl)-4-propylazepan-2-one; 4-(1H-imidazol-4-ylmethyl)-4,6-dihydro-5H-thieno[3,2-b]pyrrol-5-one; 2-(5-oxo-5,6-dihydro-4H-thieno[3,2-b]pyrrol-4-yl)acetamide; 4-{[2-(trifluoromethyl)imidazo[1,2-a]pyridin-3-yl]methyl}-4,6-dihydro-5H-thieno[3,2-b]pyrrol-5-one; 4-{[2-(trifluoromethyl)imidazo[1,2-a]pyridin-3-yl]methyl}hexahydro-5H-thieno[3,2-b]pyrrol-5-one; 1-(1H-imidazol-4-ylmethyl)-1H-thieno[3,4-b]pyrrol-2(3H)-one; 2-(6-chloro-2-oxo-1,3-benzothiazol-3(2H)-yl)acetamide; 6-bromo-3-(1H-imidazol-1-ylmethyl)-1,3-benzothiazol-2(3H)-one; 2-(6-bromo-2-oxo-1,3-benzothiazol-3(2H)-yl)propanamide; 2-(6-bromo-2-oxo-1,3-benzothiazol-3(2H)-yl)propanamide; 2-(6-fluoro-2-oxo-1,3-benzothiazol-3(2H)-yl)acetamide; 2-(6-methyl-2-oxo-1,3-benzothiazol-3(2H)-yl)acetamide; 6-fluoro-3-(1H-imidazol-1-ylmethyl)-1,3-benzoxazol-2(3H)-one; 1-(1H-imidazol-4-ylmethyl)pyrazolo[1,5-a]pyridin-2(1H)-one; 2-(6-chloro-3-oxo-3,4-dihydroisoquinolin-2(1H)-yl)propanamide; 5-chloro-2-(1H-imidazol-4-ylmethyl)-1,4-dihydroisoquinolin-3(2H)-one; 2-(6-chloro-2-oxo-3,4-dihydroquinolin-1(2H)-yl)acetamide; 2-(6-bromo-2-oxo-3,4-dihydroquinolin-1(2H)-yl)acetamide; 1-(1H-imidazol-4-ylmethyl)-3,4-dihydroquinolin-2(1H)-one; 2-(6-iodo-2-oxo-3,4-dihydroquinolin-1(2H)-yl)acetamide; 2-(6-cyano-2-oxo-3,4-dihydroquinolin-1(2H)-yl)acetamide; 7-chloro-2-{[2-(trifluoromethyl)imidazo[1,2-a]pyridin-3-yl]methyl}-1,2,4,5-tetrahydro-3H-2-benzazepin-3-one; 7-chloro-2-(1H-imidazol-4-ylmethyl)-1,2,4,5-tetrahydro-3H-2-benzazepin-3-one; 7-chloro-3-(1H-imidazol-4-ylmethyl)-1,3,4,5-tetrahydro-2H-3-benzazepin-2-one; 7-chloro-3-{[2-(trifluoromethyl)imidazo[1,2-a]pyridin-3-yl]methyl}-1,3,4,5-tetrahydro-2H-3-benzazepin-2-one; 1-[(5-fluoro-2-phenyl-1H-indol-1-yl)methyl]piperidin-2-one; 1-[(2-phenyl-1H-indol-1-yl)methyl]piperidin-2-one; [(2E)-1-(1H-imidazol-4-ylmethyl)-4-propylpyrrolidin-2-ylidene]cyanamide; 2-[(2E)-2-(cyanoimino)-4-propylpyrrolidin-1-yl]acetamide; 2-[(2E)-2-(cyanoimino)-4-propylpyrrolidin-1-yl]propanamide; 2-[(2E)-2-(cyanoimino)-4-propylpyrrolidin-1-yl]butanamide; and N-[(2E)-1-(1H-imidazol-4-ylmethyl)-4-propylpyrrolidin-2-ylidene]methanesulfonamide.

Most preferred compounds of the present invention are those selected from the group consisting of: 1-(1H-imidazol-4-ylmethyl)-5-propylpiperidin-2-one; 1-(1H-imidazol-1-ylmethyl)-5-propylpiperidin-2-one; 1-(imidazo[1,2-a]pyridin-3-ylmethyl)-5-propylpiperidin-2-one; 1-(1H-imidazol-1-ylmethyl)-5-phenylpiperidin-2-one; 1-(imidazo[1,2-a]pyridin-3-ylmethyl)-4-phenylpiperidin-2-one; 1-(1H-imidazol-1-ylmethyl)-4-phenylpiperidin-2-one; 1-(imidazo[1,2-a]pyridin-3-ylmethyl)-4-propylpiperidin-2-one; 1-(1H-imidazol-5-ylmethyl)-4-propylpiperidin-2-one; 1-(1H-imidazol-1-ylmethyl)-4-propylpiperidin-2-one; 1-(1H-imidazol-4-ylmethyl)-1H-thieno[3,4-b]pyrrol-2(3H)-one; 6-bromo-3-(1H-imidazol-1-ylmethyl)-1,3-benzothiazol-2(3H)-one; 2-(6-bromo-2-oxo-1,3-benzothiazol-3(2H)-yl)propanamide; 5-chloro-2-(1H-imidazol-4-ylmethyl)-1,4-dihydroisoquinolin-3(2H)-one; 1-[(5-fluoro-2-phenyl-1H-indol-1-yl)methyl]piperidin-2-one; and [(2E)-1-(1H-imidazol-4-ylmethyl)-4-propylpyrrolidin-2-ylidene]cyanamide.

The compounds of the present invention are for use as a medicament, in particular for disorder is selected from the group consisting of epilepsy, epileptogenesis, seizure disorders, convulsions, Parkinson's disease, dyskinesia induced by dopamine replacement therapy, tardive dyskinesia induced by administration of neuroleptic drugs, Huntington Chorea, and other neurological disorders including bipolar disorders, mania, depression, anxiety, panic disorders, attention deficit hyperactivity disorder (ADHD), migraine, trigeminal and other neuralgia, chronic pain, neuropathic pain, cerebral ischemia, cardiac arrhythmia, myotonia, cocaine abuse, stroke, myoclonus, tremor, essential tremor, simple or complex tics, Tourette syndrome, restless leg syndrome and other movement disorders, neonatal cerebral haemorrhage, amyotrophic lateral sclerosis, spasticity and degenerative diseases, subjective tinnitus, apathy syndrome; bronchial asthma, asthmatic status and allergic bronchitis, asthmatic syndrome, bronchial hyperreactivity and bronchospastic syndromes, lower urinary tract disorders, as well as allergic and vasomotor rhinitis and rhinoconjunctivitis.

Specific disorders are epilepsy, dyskinesia induced by dopamine replacement therapy, chronic pain, neuropathic pain.

A further aspect of the present invention relates to a pharmaceutical composition comprising an effective amount of a compound of formula (I) in combination with a pharmaceutically acceptable diluent or carrier.

The following paragraphs provide definitions of the various chemical moieties that make up the compounds according to the invention and are intended to apply uniformly through-out the specification and claims unless an otherwise expressly set out definition provides a broader definition.

“C₁₋₆ alkyl” refers to alkyl groups having 1 to 6, or 1 to 4 carbon atoms. This term is exemplified by groups such as methyl, ethyl, n-propyl, isopropyl, n-butyl, isobutyl, tert-butyl, n-pentyl, n-hexyl, trifluoromethyl and the like.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.

“Heterocycle” refers to a saturated or unsaturated ring system containing, in addition to carbon atoms, at least one hetero atom, such as nitrogen, oxygen and/or sulfur. “Heterocycle” includes both “heteroaryl” and “heterocycloalkyl”.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl,1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazopyridinyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl,benzoquinolyl, imidazopyrimidinyl, imidazopyridazinyl, imidazothiazolyl or imidazothiadiazolyl.

“C₂₋₆ alkenyl” refers to alkenyl groups preferably having from 2 to 6 carbon atoms and having at least 1 or 2 sites of alkenyl unsaturation. Preferable alkenyl groups include ethenyl (vinyl, —CH═CH₂), n-2-propenyl (allyl, —CH₂CH═CH₂) and the like.

“C₂₋₆ alkynyl” refers to alkynyl groups preferably having from 2 to 6 carbon atoms and having at least 1-2 sites of alkynyl unsaturation, preferred alkynyl groups include ethynyl(—C≡CH), propargyl(—CH₂C≡CH), and the like.

“C₃₋₈ cycloalkyl” refers to a saturated carbocyclic group of from 3 to 8 carbon atoms having a single ring (e.g., cyclohexyl) or multiple condensed rings (e.g., norbornyl).

Preferred cycloalkyl include cyclopropyl, cyclobutyl, cyclopentyl, cyclohexyl, norbornyl and the like.

“Heterocycloalkyl” refers to a C₃₋₈ cycloalkyl group according to the definition above, in which 1 to 3 carbon atoms are replaced by hetero atoms chosen from the group consisting of O, S, NR, R being defined as hydrogen or C₁₋₆ alkyl.

“Alkoxy” refers to the group —O—R where R includes “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”.

“Amino” refers to the group —NRR′ where each R, R′ is independently hydrogen, “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, and where R and R′, together with the nitrogen atom to which they are attached, can optionally form a 3-8-membered heterocycloalkyl ring.

“Amido” refers to the group —C(═O)NRR′ where each R, R′ is independently hydrogen, “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, and where R and R′, together with the nitrogen atom to which they are attached, can optionally form a 3-8-membered heterocycloalkyl ring.

“Acylamino” refers to the group —NRC(O)R′ wherein R and R′ are as defined hereabove for the amino group.

“Ureido” refers to the group —NR″C(O)NRR′ wherein R and R′ are as defined hereabove for the amino group, and R″ is as defined hereabove.

“Sulfanyl” refers to the group —SR where R is “C₁₋₆ alkyl”, “C₂₋₆ alkenyl”, “C₂₋₆ alkynyl”, “C₃₋₈ cycloalkyl”, “heterocycloalkyl”, “aryl” or “heteroaryl”.

“Sulfinyl” refers to the group —S(═O)R where R is “C₁₋₆ alkyl”, “C₂₋₆ alkenyl”, “C₂₋₆ alkynyl”, “C₃₋₈ cycloalkyl”, “heterocycloalkyl”, “aryl” or “heteroaryl”.

“Sulfonyl” refers to the group —S(═O)₂R where R is “C₁₋₆ alkyl”, “C₂₋₆ alkenyl”, “C₂₋₆ alkynyl”, “C₃₋₈ cycloalkyl”, “heterocycloalkyl”, “aryl” or “heteroaryl”.

“Alkylsulfonyl” refers to the group —S(═O)₂R wherein R is an alkyl moiety, e.g. a “C₁₋₆ alkyl”.

“Halogen” refers to fluoro, chloro, bromo and iodo atoms.

“Substituted or unsubstituted”: Unless otherwise constrained by the definition of the individual substituent, the above set out groups, like “alkyl”, “alkenyl”, “alkynyl”, “aryl” and “heteroaryl” etc. groups can optionally be substituted with from 1 to 5 substituents selected from the group consisting of “C₁₋₆ alkyl”, “C₂₋₆ alkenyl”, “C₂₋₆ alkynyl”, “cycloalkyl”, “heterocycloalkyl”, “amino”, “amido”, “acylamino”, “ureido”, “aryl”, “heteroaryl”, “alkoxy”, “halogen”, cyano, hydroxy, mercapto, nitro, “amido”, “sulfanyl”, “sulfinyl”, “sulfonyl” and the like.

The “pharmaceutically acceptable salts” according to the invention include therapeutically active, non-toxic acid or base salt forms which the compounds of formula (I) are able to form.

The acid addition salt form of a compound of formula (I) that occurs in its free form as a base can be obtained by treating the free base with an appropriate acid such as an inorganic acid, for example, a hydrohalic such as hydrochloric or hydrobromic, sulfuric, nitric, phosphoric and the like; or an organic acid, such as, for example, acetic, trifluoroacetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, pamoic and the like.

The compounds of formula (I) containing acidic protons may be converted into their therapeutically active, non-toxic base addition salt forms, e.g. metal or amine salts, by treatment with appropriate organic and inorganic bases. Appropriate base salt forms include, for example, ammonium salts, alkali and earth alkaline metal salts, e.g. lithium, sodium, potassium, magnesium, calcium salts and the like, salts with organic bases, e.g. N-methyl-D-glucamine, hydrabamine salts, and salts with amino acids such as, for example, arginine, lysine and the like.

Conversely said salt forms can be converted into the free forms by treatment with an appropriate base or acid.

Compounds of the formula (I) and their salts can be in the form of a solvate, which is included within the scope of the present invention. Such solvates include for example hydrates, alcoholates and the like.

Many of the compounds of formula (I) and some of their intermediates have at least one stereogenic center in their structure. This stereogenic center may be present in a R or a S configuration, said R and S notation is used in correspondence with the rules described in Pure Appl. Chem., 45 (1976) 11-30.

The invention also relates to all stereoisomeric forms such as enantiomeric and diastereoisomeric forms of the compounds of formula (I) or mixtures thereof (including all possible mixtures of stereoisomers).

With respect to the present invention reference to a compound or compounds is intended to encompass that compound in each of its possible isomeric forms and mixtures thereof, unless the particular isomeric form is referred to specifically.

Compounds according to the present invention may exist in different polymorphic forms. Although not explicitly indicated in the above formula, such forms are intended to be included within the scope of the present invention.

Some of the compounds of formula (I) may also exist in tautomeric forms. Such forms although not explicity indicated in the above formula are intended to be included within the scope of the present invention.

The invention also includes within its scope pro-drug forms of the compounds of formula (I) and its various sub-scopes and sub-groups.

The compounds of formula (I) according to the invention can be prepared analogously to conventional methods as understood by the person skilled in the art of synthetic organic chemistry.

1. Synthesis of Compounds of Formula I-A.

According to one embodiment, some compounds having the general formula I-A wherein R^{4a} is H, R¹ and R³ having the same definitions as above for compounds of formula I-A, may be prepared by transformation of a compound of formula II into the corresponding amide of formula A-1, reaction of this amide with formaldehyde and deprotection according to the equation:

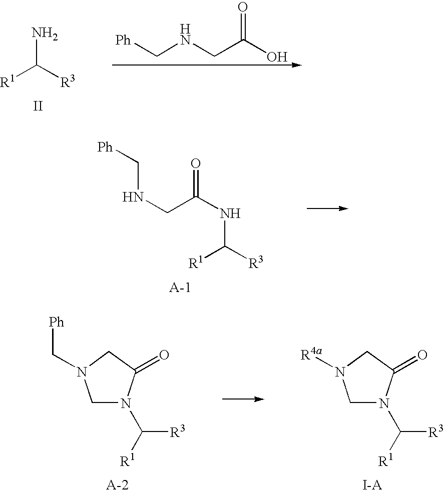

wherein R¹ and R³ have the same definitions as above for compounds of formula I-A.
Amides of formula A-1 may be obtained from amines of formula II and N-benzylglycine, or any suitable protected glycine derivative, under conventional peptide synthesis conditions, using for example N,N′-dicyclohexylcarbodiimide as a coupling agent.
Compounds of formula A-2 may be prepared by reaction of an amide of formula A-1 with formaldehyde, for example by heating an amide of formula A-1 in aqueous formaldehyde at a temperature comprised between 20 and 80° C., or according to any conventional method known to the person skilled in the art.
Imidazolidinones of formula I-A wherein R^{4a} is H may be prepared by deprotection of a compound of formula A-2 according to any conventional method known to the person skilled in the art.

According to another embodiment, some compounds of formula I-A wherein R^{4a} is an activated aromatic group may be prepared by reaction of a compound of formula I-A wherein R^{4a} is H with a compound of formula R^{4a}—F according to the equation:

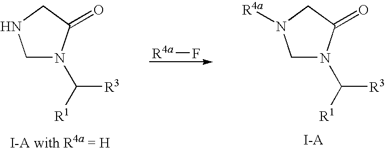

wherein R¹ and R³ have the same definitions as above for compounds of formula I-A.

This reaction may be carried out in DMSO or EtOH, between 0° C. and 60° C., in the presence of an inorganic base, for example K₂CO₃.

According to another embodiment, some compounds having the general formula I-A wherein R^{4a} is an aniline may be prepared by conventional reduction of the corresponding compound of formula I-A wherein R^{4a} is a nitrophenyl. This transformation may be performed according to conditions described by Cristau, P. et al. in Tetrahedron (2003), 59 (40), 7859-7870.

According to another embodiment, some compounds having the general formula I-A wherein R^{4a} is a phenyl moiety may be prepared by reduction of the corresponding compound of formula I-A wherein R^{4a} is an aniline. This reaction may be carried out using the conditions described by Van Loon, A. et al. in Recl. Tray. Chim. Pays-Bas (1960), 79, 977.

2. Synthesis of Compounds of Formula I-B1.

According to another embodiment, some compounds having the general formula I-B1 may be prepared by transformation of a compound of formula B1-1 into the corresponding thiazolidinone of formula IV-B1 and subsequent reaction with a compound of formula III according to the equation:

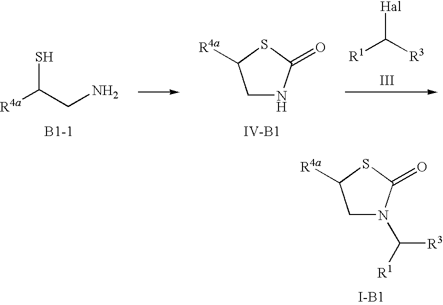

wherein Hal is a halogen atom, preferably Br, and R¹, R³ and R^{4a} are defined as hereabove for compounds of formula I-B1.
Intermediates of formula B1-1 may be prepared following the procedure described by Gaupp, S. and Effenberger, F. in Tetrahedron: Asymmetry (1999), 10 (9), 1777-1786.
Cyclization of aminothiols of formula B1-1 into thiazolidinones of formula IV-B1 is performed in the presence of phosgene and an inorganic base, in a mixture of solvents such as toluene/water.
Compounds of formula I-B1 may be obtained by alkylation of a thiazolidinone of formula IV-B1 with a compound of formula III. This reaction may be carried out with a strong base, preferably NaH or KOH, in an inert solvent such as DMF, THF or acetone at a temperature between 0° C. and 60° C.

According to another embodiment, some compounds of formula I-B1 wherein R^{4a} is —CH₂CF₃ may be prepared by transformation of a compound of formula B1-2 into the corresponding thiazolidinone of formula I-B1 according to the equation

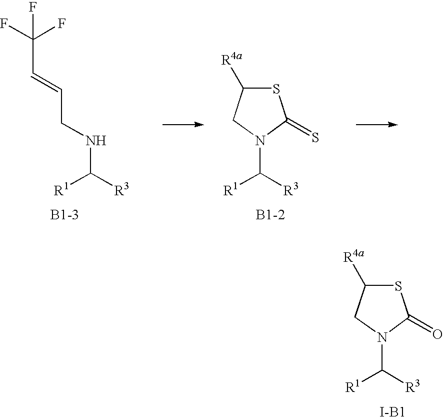

wherein R^{4a} is —CH₂CF₃, R¹ and R³ having the same definitions as described above for compounds of formula I-B1.
Compounds of formula B1-3 may be prepared as follows:

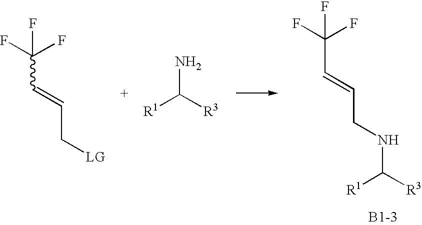

wherein LG is a suitable leaving group, including halogen, —OC(O)alkyl, —OSO₂-C₆H₄—CH₃, —OSO₂—C₆H₄—Br, —OSO₂—C₆H₄—NO₂, —OSO₂—CH₃, —OSO₂—CF₃, —OSO₂—C₄F₉, —OSO₂—CH₂—CF₃, —OSO₂—(CH₂)ₙ—N⁺Me₃, —OSO₂—F or —OClO₃.
Compounds of formula B1-2 wherein R^{4a} is —CH₂CF₃ may be prepared by treating a compound of formula B1-3 with carbon disulfide and an inorganic base such as Cs₂CO₃, in an inert solvent such as DMF and at room temperature.
Compounds of formula I-B1 may be prepared from thiazolidine thiones of formula B1-2 using potassium permanganate and benzoic acid under the conditions described by Aitken, R. A. et al. in Synthesis (1997), 7, 787-791.

3. Synthesis of Compounds of Formula I-B2.

According to another embodiment, some compounds having the general formula I-B2 wherein X is S may be prepared by transformation of a compound of formula V into the corresponding thiocarbonate of formula VIII-B2 followed by condensation with an amine of formula II according to the equation:

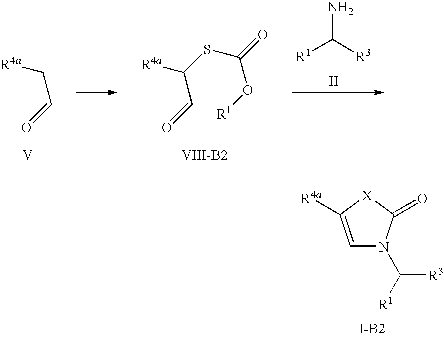

wherein X is S, R¹, R^{4a} and R³ having the same definitions as described above for compounds of formula I-B2.
Compounds of formula VIII-B2 may be prepared by reaction of an aldehyde of formula V and methoxycarbonylsulfenylchloride in CHCl₃ at room temperature as described by Sanemitsu, Y. et al. in J. Org. Chem. (1992), 57 (3), 1053-1056.
Compounds of formula I-B2 may be prepared by condensation of a compound of formula VIII-B2 with a compound of formula II in toluene under acid catalysis, for example in the presence of p-toluenesulfonic acid.

According to another embodiment, some compounds having the general formula I-B2 wherein X is S and R^{4a} is —CH₂R^{4c} may be prepared by transformation of a compound of formula B2-3 into the corresponding thiocarbamate of formula B2-2 followed by reduction/dehydration according to the equation:

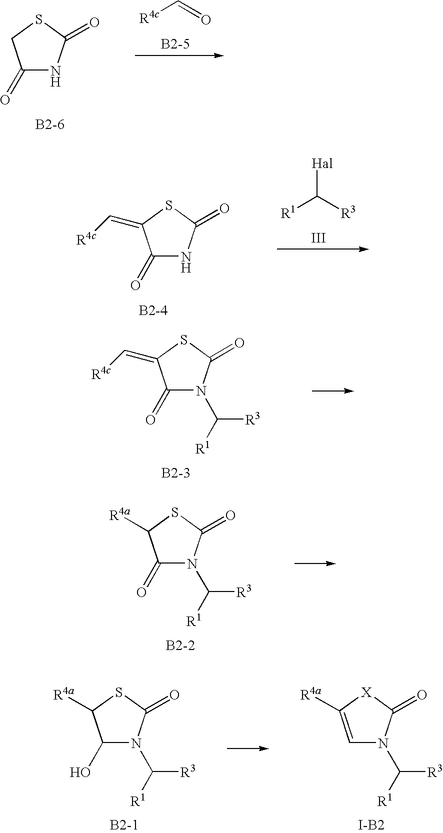

wherein R^{4a} is —CH₂R^{4c}, R^{4c} is hydrogen or C₁₋₅ alkyl optionally substituted by halogen or C₁₋₄ alkoxy, X is S, Hal is halogen, preferably Br, and R³ has the same definitions as described above for compounds of formula I-B2.
Compounds of formula B2-4 may be prepared from a thiazolidine-dione of formula B2-6 and an aldehyde of formula B2-5 (both available from commercial sources) by heating in acetic acid in the presence of sodium acetate according to the procedure described in Dundar, B. et al. in Pharmazie (2002), 57 (7), 438-441.
Compounds of formula B2-3 may be obtained by alkylation of a compound of formula B2-4 with a compound of formula Ill. This reaction may be carried out with an inorganic base such as K₂CO₃, in an inert solvent such as DMF, at a temperature comprised between 0° C. and 35° C.
Compounds of formula B2-2 may be prepared by hydrogenation of a compound of formula B2-3. This transformation may be performed according to any method known to the person skilled in the art.
Reduction of the carbonyl group of compounds B2-2 to compounds of formula B2-1 may be performed according to any method known to the person skilled in the art.
Compounds of formula I-B2 may be obtained by dehydration of compounds of formula B2-1, for example by refluxing in acetic acid.

4. Synthesis of Compounds of Formula I-B3.

According to another embodiment, some compounds having the general formula I-B3 may be prepared by transformation of a compound of formula II into the corresponding thiocarbamate of formula B3-1 followed by cyclization according to the equation:

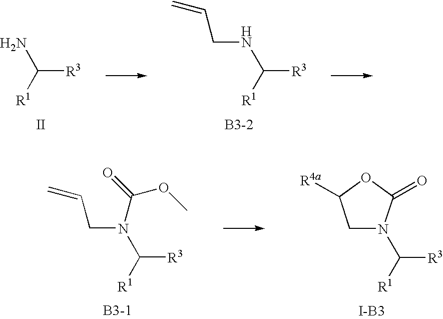

wherein R¹, R^{4a} and R³ have the same definitions as described above for compounds of formula I-B3.
Compounds of formula B3-2 may be prepared by alkylating compounds of formula II with allylbromide. This alkylation may be performed as described for the preparation of compounds of formula B2-2.
Compounds of formula B3-1 may be obtained by treatment of compounds of formula B3-2 with methylchloroformate in CH₂Cl₂, between 0° C. and room temperature, in the presence of an inorganic base such as K₂CO₃.
Compounds of formula I-B3 may be prepared by treating compounds of formula B3-1 with iodine and potassium iodide in CH₂Cl₂ and at room temperature.

5. Synthesis of Compounds of Formula I-C.

According to another embodiment, some compounds having the general formula I-C wherein R^{4a} is H may be prepared by transformation of a compound of formula II into the corresponding pyrrol of formula C-1 followed by oxydation according to the equation:

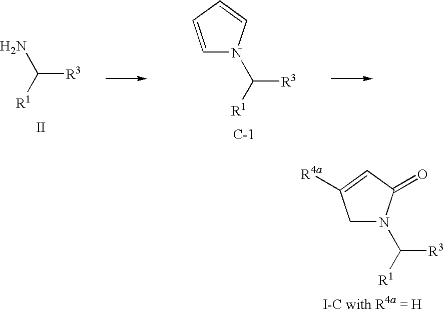

wherein R¹ and R³ have the same definitions as described above for compounds of formula I-C.
Pyrroles of formula C-1 may be obtained by refluxing an amine of formula II and 2,5-dimethoxytetrahydrofuran in acetic acid.
Compounds of formula I-C may be prepared by oxidation of pyrroles of formula C-1 with m-CPBA. This oxidation step may be performed in refluxing chloroform in the presence of an inorganic base such as K₂CO₃.

According to another embodiment, some compounds having the general formula I-C wherein R^{4a} is —CH₂R^{4d} may be prepared by transformation of a compound of formula C-2 according to the equation:

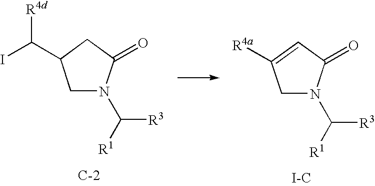

wherein R¹ and R³ have the same definitions as described above for compounds of formula I-C, R^{4a} is —CH₂R^{4d} and R^{4d} is H or C₁₋₅ alkyl.
Compounds of formula C-2 may be prepared as described in Kenda B. et al in J. Med. Chem. (2004), 47, 530, or in PCT patent applications WO 01/62726 and WO 2006/128692.
Compounds of formula I-C may be obtained by treatment of a compound of formula C-2 with a base, preferably 1,8-diazabicyclo[5.4.0]undec-7-ene, in an inert solvent such as DMF, at a temperature comprised between 20° C. and 90° C.

According to another embodiment, some compounds having the general formula I-C may be prepared by transformation of compound of formula C-3 according to the equation:

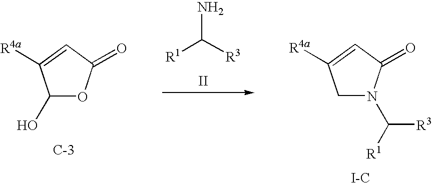

wherein R¹, R³ and R^{4d} have the same definitions as described above for compounds of formula I-C.
Compounds of formula C-3 may be prepared as described in Kenda B. et al in J. Med. Chem. (2004), 47, 530 or in PCT patent applications WO 01/62726 and WO 2006/128692.
Compounds of formula I-C may be prepared by reductive amination of a hydroxylactone of formula C-3 with an amine of formula II as described in PCT patent applications WO 01/62726 and WO 2006/128692.

6. Synthesis of Compounds of Formula I-D1.

According to another embodiment, some compounds having the general formula I-D1 may be prepared by alkylation of a compound of formula IV-D1 according to the equation:

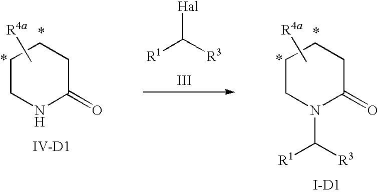

wherein R¹, R^{4a} and R³ have the same definitions as described above for compounds of formula I-D1 and Hal is halogen, preferably Br.
Compounds of formula IV-D1 are available from commercial sources or may be prepared as described by Johnson, T. A. et al. in J. Am. Chem. Soc. (2002), 124 (39),11689-11698; by Milewska, M. J. et al. in Synthesis (1996), 12, 1485-1488; and by Burger, A. and Hofstetter, A. in J. Org. Chem. (1959), 24, 1290; or according to any other method known to the person skilled in the art.
Compounds of formula I-D1 may be prepared by alkylation of a compound of formula III with a compound of formula IV-D1. This reaction may be performed as described for the synthesis of compounds of formula I-B1.

According to another embodiment, some compounds having the general formula I-D1 wherein R^{4a} is an imidazolyl, an imidazopyridinyl or an imidazopyridazinyl may be prepared by N-alkylation of compound of formula IV-D1 according to the equation:

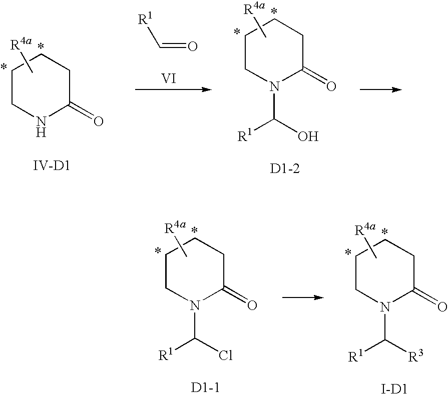

wherein R¹ and R³ have the same definitions as described above for compounds of formula I-D1.
Compounds of formula D1-2 may be prepared by hydroxyalkylation of a compound of formula IV-D1 with a carbonyl derivative of formula VI as described in PCT patent applications WO 2005/054188 and WO 2006/128692. This reaction may be carried out by heating a lactam of formula IV-D1 with an aldehyde of formula VI (or its synthetic equivalent such as paraformaldehyde in the case of formaldehyde) eventually in the presence of a base such as KOH and in a solvent such as water or a solvent mixture such as MeOH/water.
The synthesis of intermediates of formula D1-1 may be carried out using thionylchloride in toluene at a temperature between 0° C. to room temperature.
Some compounds having the general formula I-D1 may be prepared by chlorination of a compound of formula D1-2 and condensation of the corresponding derivative of formula D1-1 with a heterocycle of formula R³H as described in PCT patent applications WO 2005/054188 and WO 2006/128692.

According to another embodiment, some compounds having the general formula I-D1 may be prepared by reductive amination of compound of formula IX-D1a according to the equation:

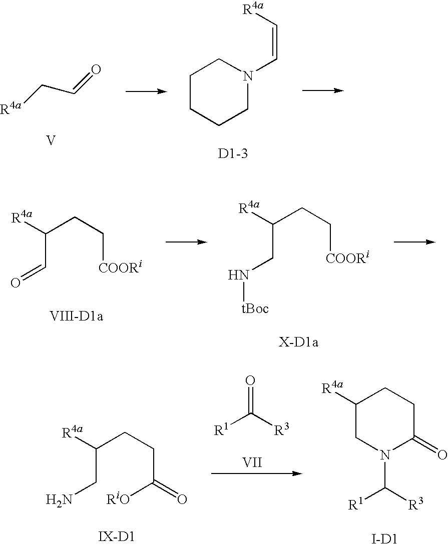

wherein R¹, R^{4a} and R³ have the same definitions as described above for compounds of formula I-D1 and Rⁱ is a C₁₋₄ alkyl.
Imines of formula D1-3 may be prepared by heating commercially available aldehydes of formula V and piperidine using conditions known to the person skilled in the art.
Compounds of formula VIII-D1a may be prepared from of formula D1-3 and ethylacrylate in an inert solvent such as acetonitrile at a temperature ranging from 0° C. to 80° C., followed by hydrolysis of the intermediate imine by heating in a mixture of acetic acid and water at a temperature of 90° C.
Compounds of formula X-D1a may be prepared by reductive amination of compounds of formula VIII-D1a with tert-butyl carbamate according to any method known to the person skilled in the art.
Compounds of formula IX-D1a can be prepared by treating compounds of formula X-D1a with a strong acid such as HCl, in a solvent such as dioxane, or according to any other method known to the person skilled in the art.
Compounds of formula I-D1 may be prepared by reductive amination of the carbonyl derivative of formula VII with an amino acid derivative of formula IX-D1a using the procedures described in PCT patent applications WO 01/62726 and WO 2006/128692.

According to another embodiment, some compounds having the general formula I-D1 wherein may be prepared by reductive amination of compound of formula VIII-D1 according to the equation:

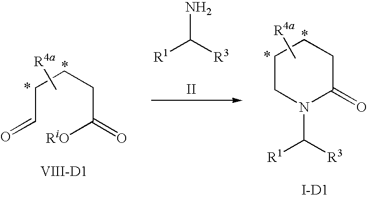

wherein R¹, R^{4a} and R³ have the same definitions as described above for compounds of formula I-D1 and Rⁱ is a C₁₋₄ alkyl.
Compounds VIII-D1 are commercially available or may be prepared as described for the synthesis of VIII-D1a.
Some compounds of formula I-D1 may be prepared by reductive amination of a carbonyl derivative of formula VIII-D1 as described in PCT patent applications WO 01/62726 and WO 2006/128692.

According to another embodiment, some compounds having the general formula I-D1 may be prepared by alkylation of compound of formula XI-D1 according to the equation:

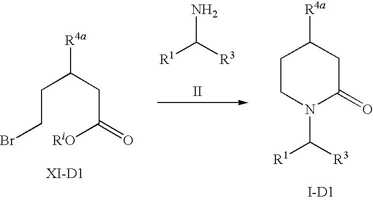

wherein R¹, R^{4a} and R³ have the same definitions as described above for compounds of formula I-D1 and Rⁱ is a C₁₋₄ alkyl.
Compounds of formula XI-D1 are commercially available or may be prepared according to the method described by Jones, J. B. and Lok, K. P. in Can. J. Chem. (1979), 57, 1025-1032 or by Burger and Hofstetter in J. Org. Chem. (1959), 24, 1290.
Compounds of formula I-D1 may be prepared by substitution of a compound of formula XI-D1 with an amine of formula II. This reaction is performed by refluxing in CH₃CN as solvent in the presence of an inorganic base such as Cs₂CO₃.

7. Synthesis of Compounds of Formula I-D2.

According to another embodiment, some compounds having the general formula I-D2 may be prepared from the protected lactam of formula XIV-D2 according to the equation:

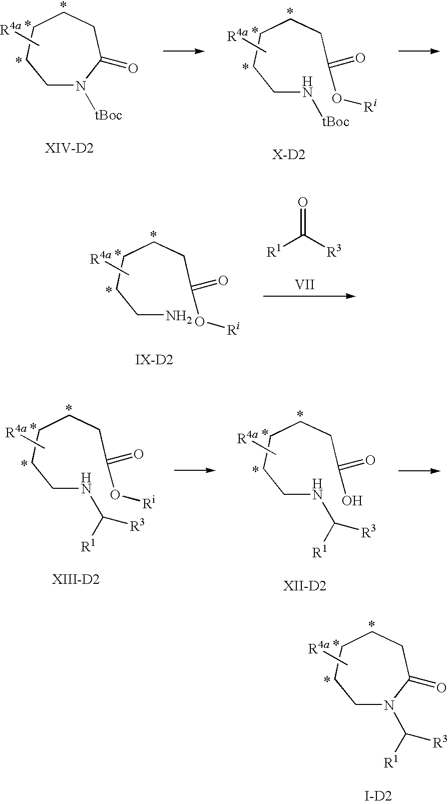

wherein R¹, R^{4a} and R³ have the same definitions as described above for compounds of formula I-D2 and Rⁱ is C₁₋₄ alkyl.
Some compounds XIV-D2 may be synthesized as a mixture of isomers according to the equation:

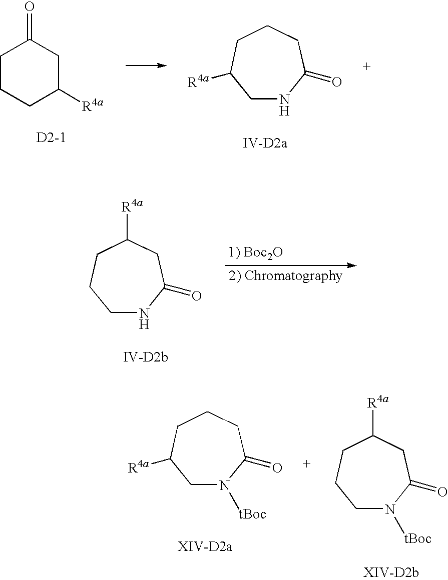

Compounds of formula D2-1 may be synthesized as described by H. O. House, W. F. Fisher in J. Org. Chem. (1969), 34, 3615-3618.
Compounds of formula IV-D2a and IV-D2b may be prepared by a Beckmann rearrangement. This transformation may be performed by treating a compound of formula D2-1 with sodiumazide and methanesulfonic acid in a solvent such as CHCl₃ and at a temperature ranging from 0° C. to room temperature.
Compounds of formula XIV-D2a and XIV-D2b may be prepared from compounds of formula IV-D2a and IV-D2b using methods known to the person skilled in the art.
Compounds of formula X-D2 may be prepared by ring opening of a tBoc-protected lactam of formula XIV-D2. This transformation may be performed by treating compounds of formula XIV-D2 with sodium methoxide in methanol at a temperature between 0° C. and 5° C.
Deprotection of compounds of formula X-D2 into IX-D2 is performed under conditions known to the person skilled in the art.
Compounds having the general formula XIII-D2 may be prepared by reductive amination of the carbonyl derivative of formula VII with a compound of formula IX-D2 according to the method described in PCT patent applications WO 01/62726 and WO 2006/128692.
Compounds of formula XII-D2 may be prepared by hydrolysis of compounds of formula XIII-D2. This transformation may be performed according to any method known to the person skilled in the art.
Compounds of formula I-D2 may be obtained from compounds of formula XII-D2 under conventional peptide synthesis conditions, by using coupling agents, for example 2-(1H-benzotriazol-1-yl)-1,1,3,3-tetramethyluronium tetrafluoroborate, or according to any other method known to the person skilled in the art.

8. Synthesis of Compounds of Formula I-F1.

According to another embodiment, some compounds having the general formula I-F1 may be prepared from the protected anilines of formula F1-2 according to the equation:

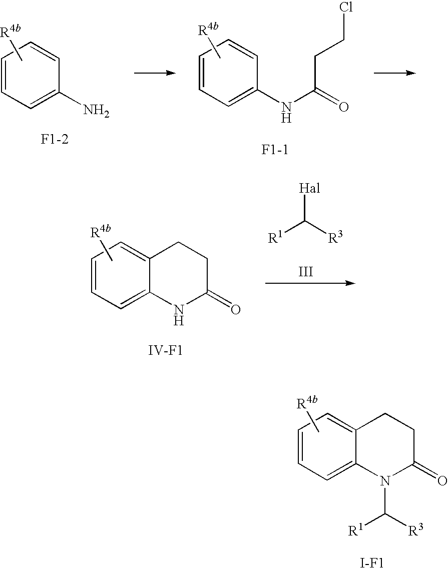

wherein R¹, R^{4b} and R³ have the same definitions as described above for compounds of formula I-F1 and Hal is a halogen atom, preferably Br.
Compounds of formula F1-1 may be prepared by acylation of anilines of formula F1-2 with 3-chloropropanoyl chloride in a solvent such as acetone, at a temperature ranging from 0° C. to 56° C.
Compounds of formula IV-F1 may be prepared by heating compounds of formula F1-1 with a Lewis acid, such as AlCl₃, at high temperature (140° C. as an example).
Compounds of formula I-F1 may be obtained by alkylation of compounds of formula IV-F1 by compounds of formula III. This reaction may be carried using the conditions described for the synthesis of I-B1.

9. Synthesis of Compounds of Formula I-F2.

According to another embodiment, some compounds having the general formula I-F2 may be prepared from the protected anilines F2-3 according to the equation:

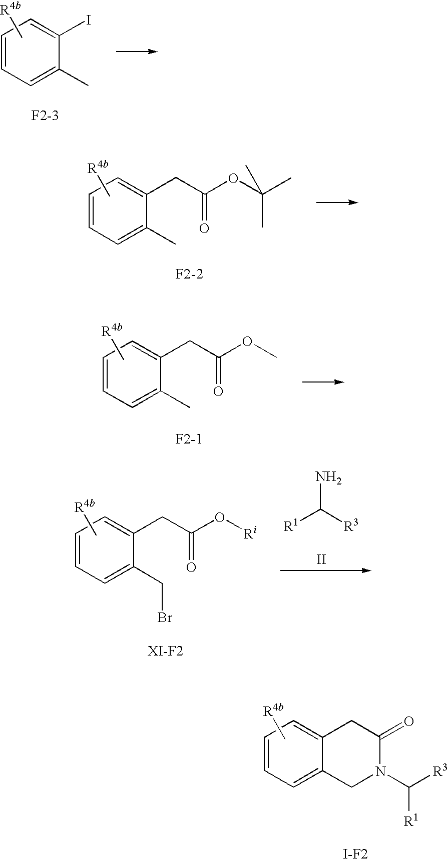

wherein R¹, R^{4b} and R³ have the same definitions as described above for compounds of formula I-F2 and Rⁱ is methyl.
Compounds of formula F2-3 are available from commercial sources.
Compounds of formula F2-2 may be prepared from compounds of formula F2-3 by treatment with t-butyl acetate in the presence of P(t-Bu) and Pd(dba)2 in a solvent such as toluene and in the presence of a base such as LiHMDS.
The transformation of compounds of formula F2-2 into compounds of formula F2-1 may be performed by transesterification in MeOH in the presence of HCl between 50 and 55° C.
Compounds of formula XI-F2 may be obtained from compounds of formula F2-1 in the presence of N-bromosuccinimide and benzoylperoxide, in an inert solvent such as benzene, and at room temperature.
Compounds of formula I-F2 may be prepared by heating compounds of formula XI-F2 and compounds II in acetonitrile at a temperature comprised between 75° C. and 80° C.

According to another embodiment, some compounds having the formula I-F2 wherein R³ is CONH₂ may be prepared by aminolysis of a compound of formula F2-4

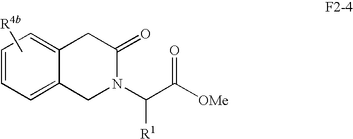

wherein R¹ and R^{4b} have the same definitions as described above for compounds of formula I-F2. This transformation may be performed in MeOH saturated with gaseous ammonia at room temperature.

10. Synthesis of Compounds of Formula I-F3.

According to another embodiment, some compounds having the general formula I-F3 may be prepared from the protected anilines of formula IV-F3 according to the equation:

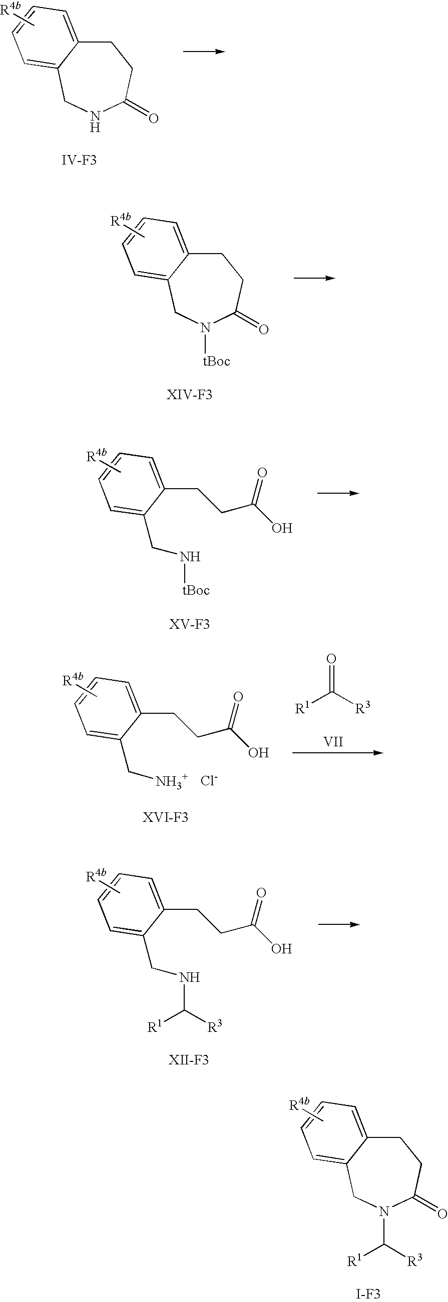

wherein R¹, R³ and R^{4b} have the same definitions as described above for compounds of formula I-F3.
Compounds of formula IV-F3 may be prepared as described by Molloy, Bryan B. in Canadian patent applications CA 1122528 and CA 1119592 and are obtained as a mixture of isomers IV-F3 and IV-F4 (see below).
Compounds of formula XIV-F3 may be prepared from benzazepines of formula IV-F3 using standard procedures known to the person skilled in the art.
Compounds of formula XV-F3 may be prepared by ring opening of compounds of formula XIV-F3. This transformation may be performed by treating compounds of io formula XIV-F3 with LiOH in a solvent such as THF.
Compounds of formula XVI-F3 may be prepared from compounds of formula XV-F3 according to any method known to the person skilled in the art.
Compounds having the general formula XII-F3 may be prepared by reductive amination of a carbonyl derivative of formula VII with a compound of formula XVI-F3, according to any method known to the person skilled in the art.
Compounds of formula I-F3 may be obtained from compounds of formula XII-F3 under conventional peptide synthesis conditions, by using coupling agents, for example 2-(1H-benzotriazol-1-yl)-1,1,3,3-tetramethyluronium tetrafluoroborate.

According to another embodiment, some compounds of formula I-F3 may be prepared by alkylation of a compound of formula IV-F3 with a compound of formula III according to the equation:

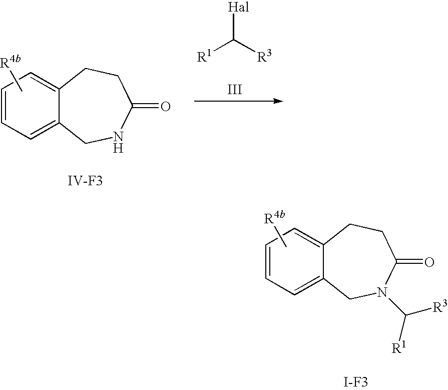

wherein R¹, R³ and R^{4b} have the same definitions as described above for compounds of formula I-F3 and Hal is a halogen, preferably Br. This reaction may be carried out in an inert solvent such as THF, in the presence of a strong base such as n-BuLi, at a temperature ranging from −70° C. to 60° C.

According to another embodiment, some compounds having the general formula I-F3 or I-F2 wherein R³ is 1H-imidazol-4-yl may be prepared from the corresponding trityl protected imidazoles of formula I-F3 or I-F4 according to the equation:

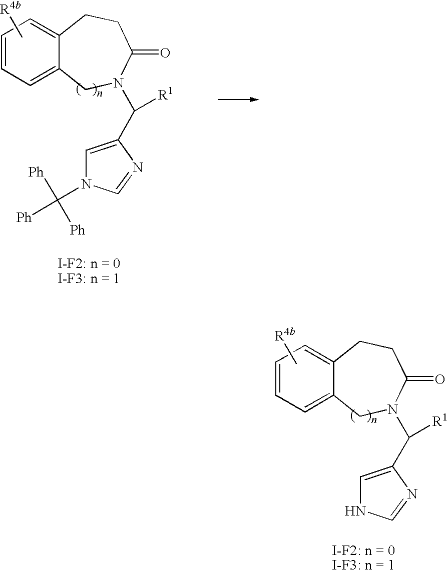

wherein R¹ and R^{4b} have the same definitions as described above for compounds of formula I-F3 or I-F2. This transformation may performed by heating the starting product with a strong acid such as HCl, eventually in the presence of a solvent such as dioxane, at a temperature ranging from room temperature to 100° C.

11. Synthesis of Compounds of Formula I-F4.

Compounds of formula I-F4 may be synthesized following the same procedure as for the synthesis of compounds of formula I-F3, using intermediate of formula IV-F4 instead of its isomer IV-F3, according to the equation:

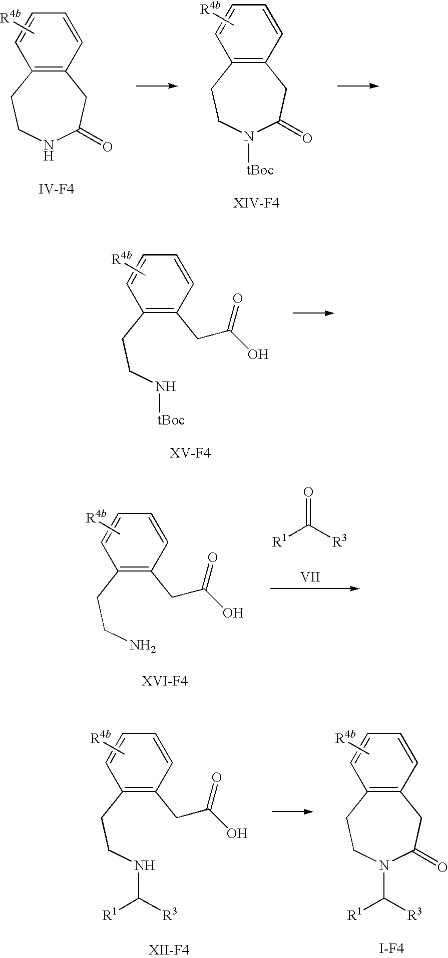

wherein R¹, R³ and R^{4b} have the same definitions as described above for compounds of formula I-F4.

12. Synthesis of Compounds of Formula I-G1.

According to another embodiment, some compounds having the general formula I-G1 may be prepared from the nitro-thiophene derivatives of formula G1-4 according to the equation:

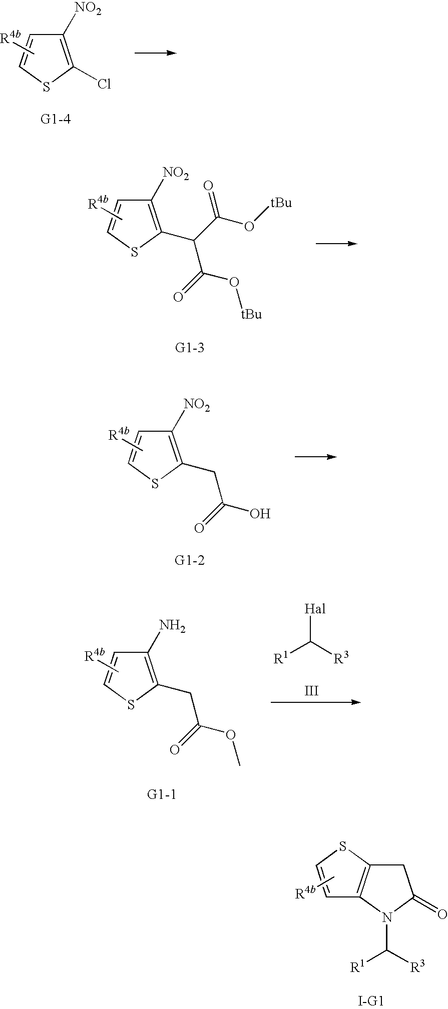

wherein R¹, R³ and R^{4b} have the same definitions as described above for compounds of formula I-G1 and Hal is a halogen atom, preferably Br.
Compounds of formula G1-3 may be prepared from compounds of formula G1-4 (available from commercial sources or synthesized using know procedure from literature) by treatment with di(tertbutyl) malonate in the presence of a strong base such as NaH, in an inert solvent, for example DMSO, at a temperature ranging from room temperature to 100° C.
Compounds of formula G1-2 may be prepared by decarboxylation of compounds of formula G1-3. This reaction is performed by refluxing compounds of formula G1-3 in TFA.
Compounds of formula G1-1 may be obtained from compounds of formula G1-2 using standard procedures known to the person skilled in the art.
Compounds of formula I-G1 may be prepared by reaction of a compound of formula G1-1 with a compound of formula III by heating with micro waves (200 W) in an inert solvent such as THF at a temperature of 100° C.

According to another embodiment, some compounds having the general formula I-G1 may be prepared from the nitro-thiophene derivatives of formula G1-3 according to the equation:

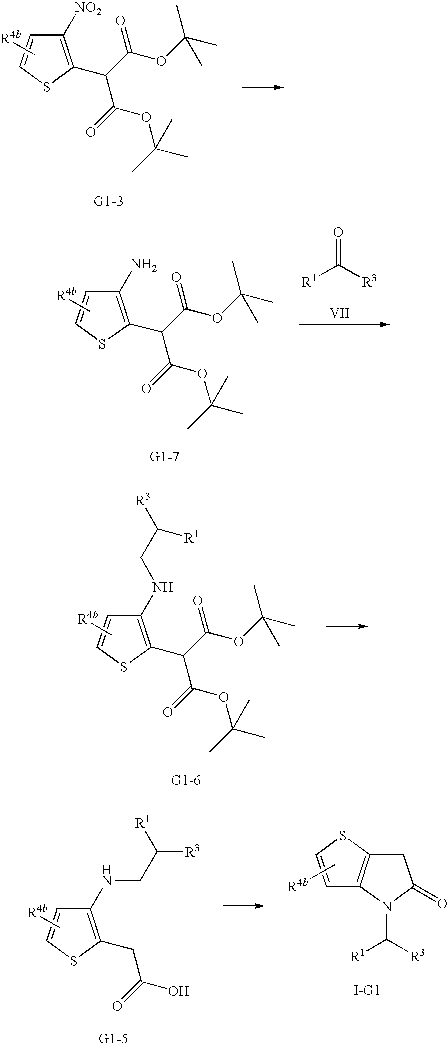

wherein R¹, R³ and R^{4b} have the same definitions as described above for compounds of formula I-G1.
Compounds of formula G1-7 may be prepared from compounds of formula G1-3 according to reduction conditions known to the person skilled in the art.
The transformation of compounds of formula G1-7 into compounds of formula G1-6 may be performed by reductive amination with a carbonyl compounds of formula VII according to the methods described in PCT patent applications WO 01/62726 and WO 2006/128692.
Compounds of formula G1-5 may be prepared by decarboxylation of compounds of formula G1-6. This reaction may be performed by heating compounds of formula G1-6 in 6N HCl at a temperature ranging from 70° C. to 75° C.
Compounds of formula I-G1 may be obtained starting from compounds of formula G1-5 under conventional peptide synthesis conditions, for example by using coupling agents such as 2-(1H-benzotriazol-1-yl)-1,1,3,3-tetramethyluronium tetrafluoroborate.

13. Synthesis of Compounds of Formula I-G2.

According to another embodiment, some compounds having the general formula I-G2 may be prepared from the thiophene G1-7 according to the equation:

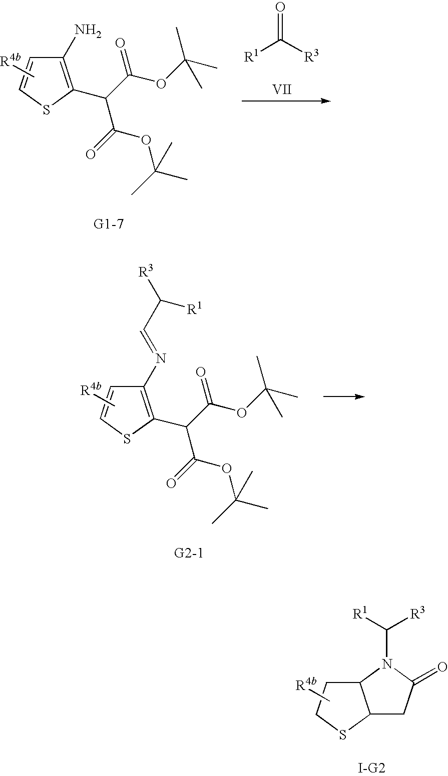

wherein R¹, R³ and R^{4b} have the same definitions as described above for compounds of formula l-G2.
The transformation of compounds of formula G1-7 into compounds of formula G2-1 may be performed in a mixture of solvents such as dichloroethane/trifluoroacetic acid, in the presence of triethylsilane, at a temperature comprised between room temperature and 40° C.
Compounds of formula I-G2 may be prepared by treating compounds of formula G2-1 with a reducing agent such as Na(CN)BH₃ in acetic acid at room temperature, or according to any other method known to the person skilled in the art.

14. Synthesis of Compounds of Formula I-G3.

According to another embodiment, some compounds having the general formula I-G3 may be prepared from compounds of formula G3-5 according to the equation:

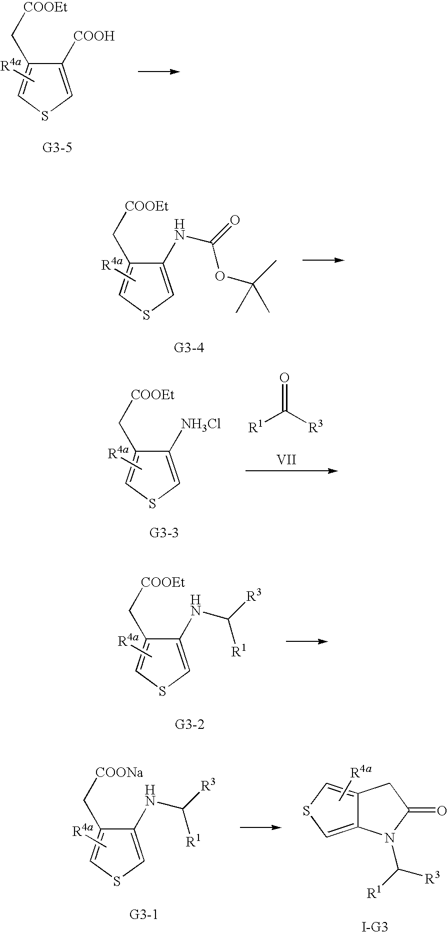

wherein R¹, R³ and R^{4b} have the same definitions as described above for compounds of formula I-G3.
Compounds of formula G3-4 may be prepared from compounds of formula G3-5 by a Curtius rearrangement. This transformation may be performed using the conditions described by Shiori, T. et al. in J. Am. Chem. Soc. (1972), 94, 6203.
Compounds of formula G3-3 may be obtained from compounds of formula G3-4 using standard procedures known to the person skilled in the art.
Compounds of formula G3-2 may be prepared using the same procedure as for the synthesis of G1-5.
Compounds of formula G3-1 may be prepared by saponification of compounds of formula G3-2 using standard procedures known to the person skilled in the art.
Compounds of formula I-G3 may be prepared from compounds of formula G3-1 under conventional peptide synthesis conditions, for example by using a coupling agent such as 2-(1H-benzotriazol-1-yl)-1,1,3,3-tetramethyluronium tetrafluoroborate (TBTU).

15. Synthesis of Compounds of Formula I-E.

According to another embodiment, some compounds having the general formula I-E may be prepared from compounds of formula IV-E according to the equation:

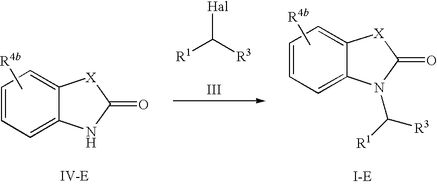

wherein R¹, R³, R^{4b} and X have the same definitions as described above for compounds of formula I-E. This reaction may be performed using the conditions described for the synthesis of compounds of formula I-B1.

According to another embodiment, some compounds having the general formula I-E wherein R¹ is hydrogen and R³ is an imidazolyl, an imidazopyridinyl or an imidazopyridazinyl may be prepared from derivatives of formula IV-E according to the equation:

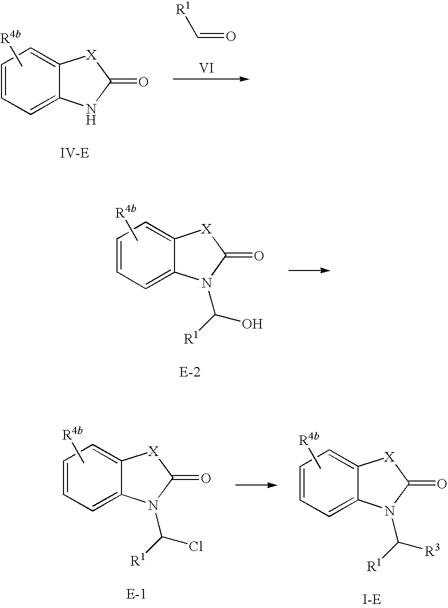

wherein R¹ is hydrogen and R³ is an imidazolyl, X and R^{4b} having the same definitions as described above for compounds of formula I-E.
Compounds of formula E-2 may be prepared by hydroxyalkylation of IV-E using the same conditions as described for the synthesis of compounds of formula D1-2.
Compounds of formula E-1 may be prepared as described for intermediates of formula D1-1.
Some compounds of formula I-E wherein R³ is imidazolyl may be prepared in one step by heating a compound E-2 with carbonyldiimidazole in a solvent such as acetonitrile.

According to another embodiment, some compounds having the general formula I-E wherein R^{4b} is Cl may be prepared by reaction of corresponding compound of formula I-E wherein R^{4b} is hydrogen with N-chlorosuccinimide in concentrated H₂SO₄, at a temperature comprised between 0° C. and room temperature.

16. Synthesis of Compounds of Formula I-H1.

According to another embodiment, some compounds having the general formula I-H1 may be prepared from compounds of formula IV-H1 according to the equation:

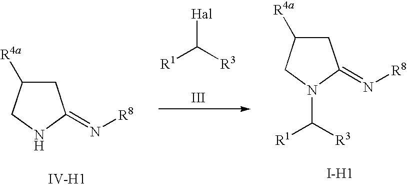

wherein Hal is halogen, preferably Br, and R¹, R³, R^{4a} and R⁸ have the same definitions as described above for compounds of formula I-H1. This reaction may be performed using the conditions described for the synthesis of compounds of formula I-B1.

According to another embodiment, some compounds having the general formula I-H1 wherein R³ is —CONH₂ may be prepared according to the equation:

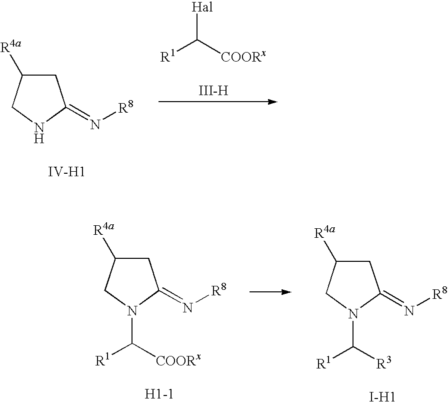

wherein Hal is halogen, preferably Br, R^{X} is C₁₋₄ alkyl and R¹, R³, R^{4a} and R⁸ have the same definitions as described above for compounds of formula I-H1.
Compounds of formula H1-1 may be prepared using the conditions described for the synthesis of compounds of formula I-B1.
Compounds of formula I-H1 may be prepared by direct ammonolysis of intermediates of formula H1-1 according to any method known to the person skilled in the art.

Compounds of formula IV-H1 wherein R⁸ is CN may be prepared according to the equation

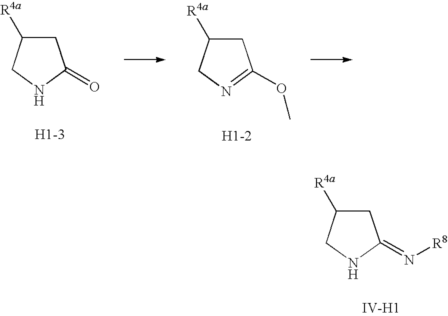

Compounds of formula H1-2 may be prepared by reaction of a compound of formula H1-3 with a trimethyloxonium borofluoride in dry dichloromethane at room temperature, or according to any method known to the person skilled in the art.
Compounds of formula IV-H1 may be prepared by reaction of a compound of formula H1-2 with cyanamide at room temperature, or according to any method known to the person skilled in the art.

Compounds of formula IV-H1 wherein R⁸ is C₁₋₆ alkylsulfonyl may be prepared by reaction of a compound of formula H1-2 with an alkylsulfonamide of formula H1-4 according to the equation:

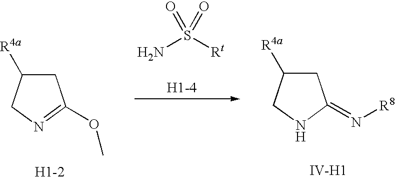

wherein R^{t} is a C₁₋₆ alkyl. This reaction may be performed in a solvent such as methanol, at reflux temperature and under anhydrous conditions, or according to any method known to the person skilled in the art.

17. Synthesis of Compounds of Formula I-H2.

According to another embodiment, some compounds having the general formula I-H2 may be prepared from compounds of formula IV-H2 according to the equation:

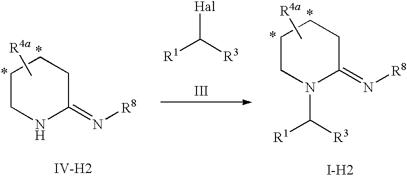

wherein Hal is halogen, preferably Br, and R¹, R³, R^{4a} and R⁸ have the same definitions as described above for compounds of formula I-H2. This reaction may be performed using the conditions described for the synthesis of compounds of formula I-B1.

According to another embodiment, some compounds having the general formula I-H2 wherein R³ is —CONH₂ may be prepared according to the equation:

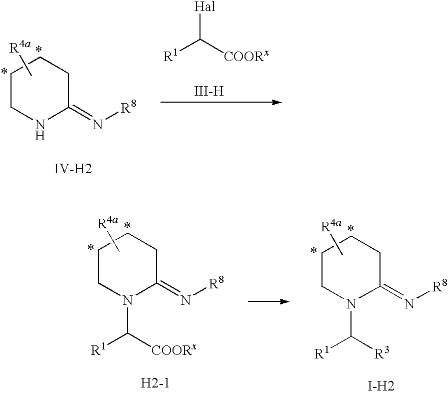

wherein Hal is halogen, preferably Br, R^{X} is C₁₋₄ alkyl and R¹, R³, R^{4a} and R⁸ have the same definitions as described above for compounds of formula I-H1.
Compounds of formula H2-1 may be prepared using the conditions described for the synthesis of compounds of formula I-B1.
Compounds of formula I-H2 may be prepared by direct ammonolysis of intermediates of formula H2-1 according to any method known to the person skilled in the art.

Compounds of formula IV-H2 wherein R⁸ is CN may be prepared according to the equation

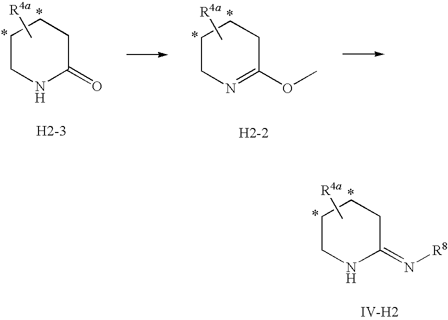

using the method described for the synthesis of compounds of formula IV-H1. Compounds of formula IV-H1 wherein R⁸ is C₁₋₆ alkylsulfonyl may be prepared by reaction of a compound of formula H1-2 with an alkylsulfonamide of formula H1-4 according to the equation:

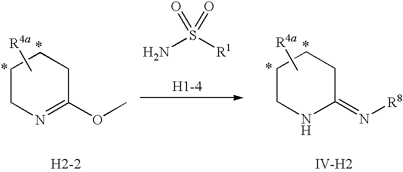

wherein R^{t} is a C₁₋₆ alkyl, using the method described for the synthesis of compounds of formula IV-H1.

18. Synthesis of Compounds of Formula I-H3.

According to another embodiment, some compounds having the general formula I-H3 may be prepared from compounds of formula IV-H3 according to the equation:

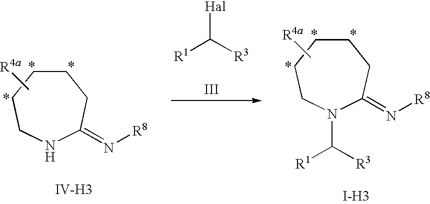

wherein Hal is halogen, preferably Br, and R¹, R³, R^{4a} and R⁸ have the same definitions as described above for compounds of formula I-H3. This reaction may be performed using the conditions described for the synthesis of compounds of formula I-B1.

According to another embodiment, some compounds having the general formula I-H3 wherein R³ is —CONH₂ may be prepared according to the equation:

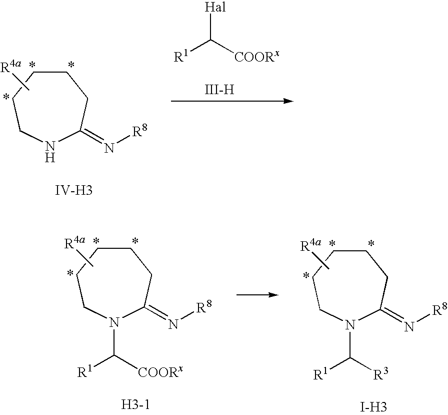

wherein Hal is halogen, preferably Br, R^{X} is C₁₋₄ alkyl and R¹, R³, R^{4a} and R⁸ have the same definitions as described above for compounds of formula I-H3.
Compounds of formula H3-1 may be prepared using the conditions described for the synthesis of compounds of formula I-B1.
Compounds of formula I-H3 may be prepared by direct ammonolysis of intermediates of formula H3-1 according to any method known to the person skilled in the art.

Compounds of formula IV-H3 wherein R⁸ is CN may be prepared according to the equation

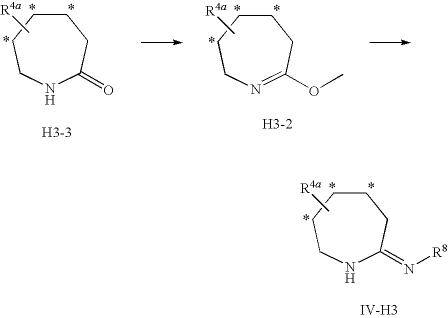

using the method described for the synthesis of compounds of formula IV-H3. Compounds of formula IV-H3 wherein R⁸ is C₁₋₆ alkylsulfonyl may be prepared by reaction of a compound of formula H3-2 with an alkylsulfonamide of formula H1-4 according to the equation:

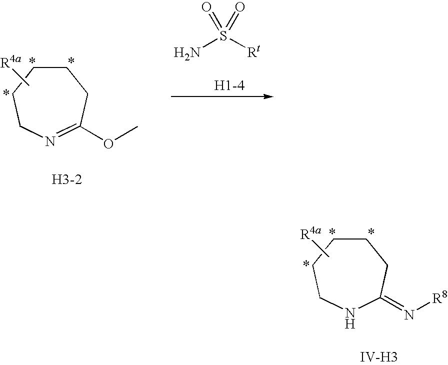

wherein R^{t} is a C₁₋₆ alkyl, using the method described for the synthesis of compounds of formula IV-H1.

According to another embodiment, the present invention consist in novel compounds selected from the group consisting of: (2S)-2-{[(benzylamino)acetyl]amino}butanamide; (2S)-2-(3-benzyl-5-oxoimidazolidin-1-yl)butanamide; (2S)-2-(5-oxoimidazolidin-1-yl)butanamide; (2S)-2-[3-(4-aminophenyl)-5-oxoimidazolidin-1-yl]butanamide; (2S)-2-(allylamino)butanamide; methyl allyl[(1S)-1-(aminocarbonyl)propyl]carbamate; (2S)-2-(1H-pyrrol-1-yl)butanamide; methyl 4-{[(1S)-1-(aminocarbonyl)propyl]amino}-3-phenylbutanoate; S-(1-formylbutyl)O-methyl thiocarbonate; 2-(2,4-dioxo-5-propyl-1,3-thiazolidin-3-yl)propanamide; 2-(4-hydroxy-2-oxo-5-propyl-1,3-thiazolidin-3-yl)propanamide; 1-cyanopentyl 4-methylbenzenesulfonate; S-[cyano(phenyl)methyl]ethanethioate; S-(1-cyanopentyl)ethanethioate; 5-butyl-1,3-thiazolidin-2-one; 5-propyl-1,3-thiazolidin-2-one; (2S)-2-[2-thioxo-5-(2,2,2-trifluoroethyl)-1,3-thiazolidin-3-yl]butanamide; 1-(hydroxymethyl)-5-phenylpiperidin-2-one; 6-fluoro-3-(hydroxymethyl)-1,3-benzoxazol-2(3H)-one; 1-(hydroxymethyl)-5-propylpiperidin-2-one; 1-(hydroxymethyl)-4-phenylpiperidin-2-one; 1-(hydroxymethyl)-4-propylpiperidin-2-one; 1-(chloromethyl)-5-phenylpiperidin-2-one; 3-(chloromethyl)-6-fluoro-1,3-benzoxazol-2(3H)-one; 1-(chloromethyl)-5-propylpiperidin-2-one; 1-(chloromethyl)-4-phenylpiperidin-2-one; ethyl 4-formylheptanoate; ethyl 4-{[(tert-butoxycarbonyl)amino]methyl}heptanoate; ethyl 4-(aminomethyl)heptanoate hydrochloride; ethyl 3-(2-bromoethyl)hexanoate; tert-butyl 2-oxo-5-propylazepane-1-carboxylate; tert-butyl 2-oxo-5-phenylazepane-1-carboxylate; 4-propylazepan-2-one; 6-propylazepan-2-one; tert-butyl 2-oxo-4-propylazepane-1-carboxylate; tert-butyl 2-oxo-6-propylazepane-1-carboxylate; methyl 4-{2-[(tert-butoxycarbonyl)amino]ethyl}heptanoate; methyl 6-[(tert-butoxycarbonyl)amino]-4-phenylhexanoate; methyl 5-{[(tert-butoxycarbonyl)amino]methyl}octanoate; methyl 6-[(tert-butoxycarbonyl)amino]-3-propylhexanoate; methyl 4-(2-aminoethyl)heptanoate hydrochloride; methyl 6-amino-4-phenylhexanoate; methyl 5-(aminomethyl)octanoate hydrochloride; methyl 6-amino-3-propylhexanoate hydrochloride; methyl 4-{2-[(1H-imidazol-4-ylmethyl)amino]ethyl}heptanoate; isopropyl 4-[2-({[2-(trifluoromethyl)imidazo[1,2-a]pyridin-3-yl]methyl}amino)ethyl]heptanoate; isopropyl 4-phenyl-6-({[2-(trifluoromethyl)imidazo[1,2-a]pyridin-3-yl]methyl}amino)hexanoate; methyl 5-{[(1H-imidazol-4-ylmethyl)amino]methyl}octanoate; methyl 6-[(1H-imidazol-4-ylmethyl)amino]-3-propylhexanoate; 4-{2-[(1H-imidazol-4-ylmethyl)amino]ethyl}heptanoic acid dihydrochloride; 4-[2-({[2-(trifluoromethyl)imidazo[1,2-a]pyridin-3-yl]methyl}amino)ethyl]heptanoic acid dihydrochloride; 4-phenyl-6-({[2-(trifluoromethyl)imidazo[1,2-a]pyridin-3-yl]methyl}amino)hexanoic acid dihydrochloride; 5-{[(1H-imidazol-4-ylmethyl)amino]methyl}octanoic acid dihydrochloride; 6-[(1H-imidazol-4-ylmethyl)amino]-3-propylhexanoic acid dihydrochloride; di-tert-butyl(3-nitro-2-thienyl)malonate; di-tert-butyl(3-amino-2-thienyl)malonate; di-tert-butyl{3-[(1H-imidazol-4-ylmethyl)amino]-2-thienyl}malonate; {3-[(1H-imidazol-4-ylmethyl)amino]-2-thienyl}acetic acid dihydrochloride; [3-({[2-(trifluoromethyl)imidazo[1,2-a]pyridin-3-yl]methylene}amino)-2-thienyl]acetic acid; [3-({[2-(trifluoromethyl)imidazo[1,2-a]pyridin-3-yl]methyl}amino)-2-thienyl]acetic acid; ethyl{4-[(tert-butoxycarbonyl)amino]-3-thienyl}acetate; ethyl(4-amino-3-thienyl)acetate hydrochloride; ethyl{4-[(1H-imidazol-4-ylmethyl)amino]-3-thienyl}acetate; sodium {4-[(1H-imidazol-4-ylmethyl)amino]-3-thienyl}acetate; 1-{(3-ethoxy-3-oxopropanoyl)[(1-trityl-1H-imidazol-4-yl)methyl]amino}pyridinium chloride; ethyl 2-oxo-1-[(1-trityl-1H-imidazol-4-yl)methyl]-1,2-dihydropyrazolo[1,5-a]pyridine-3-carboxylate-methanol (1:2); tert-butyl(5-chloro-2-methylphenyl)acetate; methyl(5-chloro-2-methylphenyl)acetate; methyl [2-(bromomethyl)-5-chlorophenyl]acetate; methyl 2-(6-chloro-3-oxo-3,4-dihydroisoquinolin-2(1H)-yl)propanoate; tert-butyl (2-chloro-6-methylphenyl)acetate; methyl (2-chloro-6-methylphenyl)acetate; methyl [2-(bromomethyl)-6-chlorophenyl]acetate; 1-[(1-trityl-1H-imidazol-4-yl)methyl]-3,4-dihydroquinolin-2(1H)-one; tert-butyl 7-chloro-3-oxo-1,3,4,5-tetrahydro-2H-2-benzazepine-2-carboxylate; tert-butyl 7-chloro-2-oxo-1,2,4,5-tetrahydro-3H-3-benzazepine-3-carboxylate; 3-(2-{[(tert-butoxycarbonyl)amino]methyl}-5-chlorophenyl)propanoic acid; (2-{2-[(tert-butoxycarbonyl)amino]ethyl}-4-chlorophenyl)acetic acid; 3-[2-(aminomethyl)-5-chlorophenyl]propanoic acid hydrochloride; [2-(2-aminoethyl)-4-chlorophenyl]acetic acid hydrochloride; 3-[5-chloro-2-({[(1-trityl-1H-imidazol-4-yl)methyl]amino}methyl)phenyl]propanoic acid; [4-chloro-2-(2-{[(1-trityl-1H-imidazol-4-yl)methyl]amino}ethyl)phenyl]acetic acid; 7-chloro-2-[(1-trityl-1H-imidazol-4-yl)methyl]-1,2,4,5-tetrahydro-3H-2-benzazepin-3-one; 7-chloro-3-[(1-trityl-1H-imidazol-4-yl)methyl]-1,3,4,5-tetrahydro-2H-3-benzazepin-2-one; 3-(chloromethyl)-2-(trifluoromethyl)-imidazo[1,2-a]pyridine; 5-methoxy-3-propyl-3,4-dihydro-2H-pyrrole; [(2E)-4-propylpyrrolidin-2-ylidene]cyanamide; ethyl 2-[(2E)-2-(cyanoimino)-4-propylpyrrolidin-1-yl]butanoate; and N-[(2E)-4-propylpyrrolidin-2-ylidene]methanesulfonamide; for their use as synthetic intermediates.

In a further aspect, the present invention concerns also the use of a compound of formula (I) or a pharmaceutically acceptable salt thereof for the manufacture of a medicament for the treatment of neurological and other disorders such as mentioned above.

In particular, the present invention concerns the use of a compound of formula (I) or a pharmaceutically acceptable salt thereof for the manufacture of a medicament for the treatment of epilepsy, Parkinson's disease, dyskinesia, migraine, tremor, essential tremor, bipolar disorders, chronic pain, neuropathic pain, or bronchial, asthmatic or allergic conditions. The compounds of the present invention may also be useful in the treatment of lower urinary tract disorders.

In a specific embodiment, the present invention concerns the use of a compound selected from the group consisting of: (2S)-2-[3-(4-nitrophenyl)-5-oxoimidazolidin-1-yl]butanamide; (2S)-2-[3-(2,4-dinitrophenyl)-5-oxoimidazolidin-1-yl]butanamide; (2S)-2-(5-oxo-3-phenylimidazolidin-1-yl)butanamide; 2-[5-(iodomethyl)-2-oxo-1,3-oxazolidin-3-yl]butanamide; 2-(2-oxo-2,5-dihydro-1H-pyrrol-1-yl)butanamide; 2-(2-oxo-4-phenyl-2,5-dihydro-1H-pyrrol-1-yl)butanamide; 2-(4-methyl-2-oxo-2,5-dihydro-1H-pyrrol-1-yl)butanamide; (+)-(2S)-2-(2-oxo-4-propyl-2,5-dihydro-1H-pyrrol-1-yl)butanamide; (2S)-2-(2-oxo-5-propyl-1,3-thiazol-3(2H)-yl)butanamide; 2-(2-oxo-5-propyl-1,3-thiazol-3(2H)-yl)propanamide; 2-(5-butyl-2-oxo-1,3-thiazolidin-3-yl)butanamide; 2-(5-butyl-2-oxo-1,3-thiazolidin-3-yl)propanamide; 2-(2-oxo-5-phenyl-1,3-thiazolidin-3-yl)propanamide; 2-(2-oxo-5-propyl-1,3-thiazolidin-3-yl)butanamide; 2-(2-oxo-5-phenyl-1,3-thiazolidin-3-yl)butanamide; 2-(2-oxo-5-propyl-1,3-thiazolidin-3-yl)propanamide; (2S)-2-[2-oxo-5-(2,2,2-trifluoroethyl)-1,3-thiazolidin-3-yl]butanamide; 1-{[6-chloro-2-(trifluoromethyl)imidazo[1,2-b]pyridazin-3-yl]methyl}piperidin-2-one; 1-(1H-imidazol-4-ylmethyl)-5-propylpiperidin-2-one; 1-(1H-imidazol-1-ylmethyl)-5-propylpiperidin-2-one; 1-(imidazo[1,2-a]pyridin-3-ylmethyl)-5-propylpiperidin-2-one; 1-(1H-imidazol-1-ylmethyl)-5-phenylpiperidin-2-one; 1-(imidazo[1,2-a]pyridin-3-ylmethyl)-5-phenylpiperidin-2-one; 1-(imidazo[1,2-a]pyridin-3-ylmethyl)-4-phenylpiperidin-2-one; 1-(1H-imidazol-1-ylmethyl)-4-phenylpiperidin-2-one; 1-(imidazo[1,2-a]pyridin-3-ylmethyl)-4-propylpiperidin-2-one; 1-(1H-imidazol-5-ylmethyl)-4-propylpiperidin-2-one; 1-(1H-imidazol-1-ylmethyl)-4-propylpiperidin-2-one; 1-{[6-chloro-2-(trifluoromethyl)imidazo[1,2-b]pyridazin-3-yl]methyl}azepan-2-one; 1-(1H-imidazol-5-ylmethyl)-5-propylazepan-2-one; 5-propyl-1-{[2-(trifluoromethyl)imidazo[1,2-a]pyridin-3-yl]methyl}azepan-2-one; 1-(1H-imidazol-5-ylmethyl)-5-phenylazepan-2-one; 5-phenyl-1-{[2-(trifluoromethyl)imidazo[1,2-a]pyridin-3-yl]methyl}azepan-2-one; 1-(1H-imidazol-5-ylmethyl)-6-propylazepan-2-one; 1-(1H-imidazol-4-ylmethyl)-4-propylazepan-2-one; 4-(1H-imidazol-4-ylmethyl)-4,6-dihydro-5H-thieno[3,2-b]pyrrol-5-one; 2-(5-oxo-5,6-dihydro-4H-thieno[3,2-b]pyrrol-4-yl)acetamide; 4-{[2-(trifluoromethyl)imidazo[1,2-a]pyridin-3-yl]methyl}-4,6-dihydro-5H-thieno[3,2-b]pyrrol-5-one; 4-{[2-(trifluoromethyl)imidazo[1,2-a]pyridin-3-yl]methyl}hexahydro-5H-thieno[3,2-b]pyrrol-5-one; 1-(1H-imidazol-4-ylmethyl)-1H-thieno[3,4-b]pyrrol-2(3H)-one; 2-(6-bromo-2-oxo-1,3-benzothiazol-3(2H)-yl)acetamide; 2-(2-oxo-1,3-benzothiazol-3(2H)-yl)acetamide; 2-(6-chloro-2-oxo-1,3-benzothiazol-3(2H)-yl)acetamide; 6-bromo-3-(1H-imidazol-1-ylmethyl)-1,3-benzothiazol-2(3H)-one; 6-bromo-3-(2-oxopropyl)-1,3-benzothiazol-2(3H)-one; 2-(6-nitro-2-oxo-1,3-benzothiazol-3(2H)-yl)acetamide; 2-(6-bromo-2-oxo-1,3-benzothiazol-3(2H)-yl)propanamide; 2-(6-bromo-2-oxo-1,3-benzothiazol-3(2H)-yl)propanamide; 2-(6-fluoro-2-oxo-1,3-benzothiazol-3(2H)-yl)acetamide; 2-(6-methyl-2-oxo-1,3-benzothiazol-3(2H)-yl)acetamide; 6-fluoro-3-(1H-imidazol-1-ylmethyl)-1,3-benzoxazol-2(3H)-one; 1-(1H-imidazol-4-ylmethyl)pyrazolo[1,5-a]pyridin-2(1H)-one; 2-(6-chloro-3-oxo-3,4-dihydroisoquinolin-2(1H)-yl)propanamide; 5-chloro-2-(1H-imidazol-4-ylmethyl)-1,4-dihydroisoquinolin-3(2H)-one; 2-(6-chloro-2-oxo-3,4-dihydroquinolin-1(2H)-yl)acetamide; 2-(6-bromo-2-oxo-3,4-dihydroquinolin-1(2H)-yl)acetamide; 1-(1H-imidazol-4-ylmethyl)-3,4-dihydroquinolin-2(1H)-one; 2-(6-iodo-2-oxo-3,4-dihydroquinolin-1(2H)-yl)acetamide; 2-(6-cyano-2-oxo-3,4-dihydroquinolin-1(2H)-yl)acetamide; 7-chloro-2-{[2-(trifluoromethyl)imidazo[1,2-a]pyridin-3-yl]methyl}-1,2,4,5-tetrahydro-3H-2-benzazepin-3-one; 7-chloro-2-(1H-imidazol-4-ylmethyl)-1,2,4,5-tetrahydro-3H-2-benzazepin-3-one; 7-chloro-3-(1H-imidazol-4-ylmethyl)-1,3,4,5-tetrahydro-2H-3-benzazepin-2-one; 7-chloro-3-{[2-(trifluoromethyl)imidazo[1,2-a]pyridin-3-yl]methyl}-1,3,4,5-tetrahydro-2H-3-benzazepin-2-one; 1-[(5-fluoro-2-phenyl-1H-indol-1-yl)methyl]piperidin-2-one; 1-[(2-phenyl-1H-indol-1-yl)methyl]piperidin-2-one; [(2E)-1-(1H-imidazol-4-ylmethyl)-4-propylpyrrolidin-2-ylidene]cyanamide; 2-[(2E)-2-(cyanoimino)-4-propylpyrrolidin-1-yl]acetamide; 2-[(2E)-2-(cyanoimino)-4-propylpyrrolidin-1-yl]propanamide; 2-[(2E)-2-(cyanoimino)-4-propylpyrrolidin-1-yl]butanamide; and N-[(2E)-1-(1H-imidazol-4-ylmethyl)-4-propylpyrrolidin-2-ylidene]methanesulfonamide.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 3 to 3000 mg, preferably 25 to 500 mg of active ingredient per unit dosage form.

The term “treatment” as used herein includes curative treatment and prophylactic treatment.

By “curative” is meant efficacy in treating a current symptomatic episode of a disorder or condition.

By “prophylactic” is meant prevention of the occurrence or recurrence of a disorder or condition.

The term “epilepsy” as used herein refers to a chronic neurologic condition characterised by unprovoked, recurrent epileptic seizures. An epileptic seizure is the manisfestation of an abnormal and excessive synchronised discharge of a set of cerebral neurons; its clinical manifestations are sudden and transient. The term “epilepsy” as used herein can also refer to a disorder of brain function characterised by the periodic occurrence of seizures. Seizures can be “nonepileptic” when evoked in a normal brain by conditions such as high fever or exposure to toxins or “epileptic” when evoked without evident provocation.

The term “seizure” as used herein refers to a transient alteration of behaviour due to the disordered, synchronous, and rhythmic firing of populations of brain neurones.

The term “Parkinsonian symptoms” relates to a syndrome characterised by slowlyness of movement (bradykinesia), rigidity and/or tremor. Parkinsonian symptoms are seen in a variety of conditions, most commonly in idiopathic parkinsonism (i.e. Parkinson's Disease) but also following treatment of schizophrenia, exposure to toxins/drugs and head injury. It is widely appreciated that the primary pathology underlying Parkinson's disease is degeneration, in the brain, of the dopaminergic projection from the substantia nigra to the striatum. This has led to the widespread use of dopamine-replacing agents (e.g. L-3,4-dihydroxyphenylalanine (L-DOPA) and dopamine agonists) as symptomatic treatments for Parkinson's disease and such treatments have been successful in increasing the quality of life of patients suffering from Parkinson's disease. However, dopamine-replacement treatments do have limitations, especially following long-term treatment. Problems can include a wearing-off of the anti-parkinsonian efficacy of the treatment and the appearance of a range of side-effects which manifest as abnormal involuntary movements, such as dyskinesias.

The term “dyskinesia” is defined as the development in a subject of abnormal involuntary movements. This appears in patients with Huntington's disease, in Parkinson's disease patients exposed to chronic dopamine replacement therapy, and in Schizophrenia patients exposed to chronic treatment with neuroleptics. Dyskinesias, as a whole, are characterised by the development in a subject of abnormal involuntary movements. One way in which dyskinesias may arise is as a side effect of dopamine replacement therapy for parkinsonism or other basal ganglia-related movement disorders.

The term “migraine” as used herein means a disorder characterised by recurrent attacks of headache that vary widely in intensity, frequency, and duration. The attacks are commonly unilateral and are usually associated with anorexia, nausea, vomiting, phonophobia, and/or photophobia. In some cases they are preceded by, or associated with, neurological and mood disturbances. Migraine headache may last from 4 hours to about 72 hours. The International Headache Society (IHS, 1988) classifies migraine with aura (classical migraine) and migraine without aura (common migraine) as the major types of migraine. Migraine with aura consists of a headache phase preceded by characteristic visual, sensory, speech, or motor symptoms. In the absence of such symptoms, the headache is called migraine without aura.

The term “bipolar disorders” as used herein refers to those disorders classified as Mood Disorders according to the Diagnostic and Statistical Manual of Mental Disorders, 4th edition (Diagnostic and Statistical Manual of Mental Disorders (DSM-IV™), American Psychiatry Association, Washington, DC, 1994). Bipolar disorders are generally characterised by spontaneously triggered repeated (i.e. at least two) episodes in which the patient's hyperexcitability, activity and mood are significantly disturbed, this disturbance consisting on some occasions of an elevation of mood and increased energy and activity (mania or hypomania), and in other occasions a lowering of mood and decreased energy and activity (depression). Bipolar disorders are separated into four main categories in the DSM-IV (bipolar (I) disorder, bipolar II disorder, cyclothymia, and bipolar disorders not otherwise specified).

The term “manic episode”, as used herein refers to a distinct period during which there is an abnormally and persistently elevated, expansive, or irritable mood with signs of pressured speech and psychomotor agitation.

The term “hypomania”, as used herein refers to a less extreme manic episode, with lower grade of severity.

The term “major depressive episode”, as used herein refers to a period of at least 2 weeks during which there is either depressed mood or the loss of interest or pleasure in nearly all activities with signs of impaired concentration and psychomotor retardation.

The term “mixed episode”, as used herein refers to a period of time (lasting at least 1 week) in which the criteria are met both for a manic episode and for a major depressive episode nearly every day.

The term “chronic pain” as used herein refers to the condition gradually being recognised as a disease process distinct from acute pain. Conventionally defined as pain that persists beyond the normal time of healing, pain can also be considered chronic at the point when the individual realises that the pain is going to be a persistent part of their lives for the foreseeable future. It is likely that a majority of chronic pain syndromes involves a neuropathic component, which is usually harder to treat than acute somatic pain.

The term “neuropathic pain” as used herein refers to pain initiated by a pathological change in a nerve which signals the presence of a noxious stimulus when no such recognisable stimulus exists, giving rise to a false sensation of pain. In other words, it appears that the pain system has been turned on and cannot turn itself off.

The term “tics” refers to common and often disabling neurological disorders. They are frequently associated with behaviour difficulties, including obsessive-compulsive disorder, attention deficit hyperactivity disorder and impulse control. Tics are involuntary, sudden, rapid, repetitive, nonrhythmic stereotype movements or vocalizations. Tics are manifested in a variety of forms, with different durations and degrees of complexity. Simple motor tics are brief rapid movements that often involve only one muscle group. Complex motor tics are abrupt movements that involve either a cluster of simple movements or a more coordinated sequence of movements. Simple vocal tics include sounds such as grunting, barking, yelping, and that clearing. Complex vocal tics include syllables, phrases, repeating other people's words and repeating one's own words.

The activity of the compounds of formula I, or their pharmaceutically acceptable salts, as anticonvulsants may be determined in the audiogenic seizure model. The objective of this test is to evaluate the anticonvulsant potential of a compound by means of audiogenic seizures induced in sound-susceptible mice, a genetic animal model with reflex seizures. In this model of primary generalised epilepsy, seizures are evoked without electrical or chemical stimulation and the seizure types are, at least in part, similar in their clinical phenomenology to seizures occurring in man (Löscher W. & Schmidt D., Epilepsy Res. (1998), 2, 145-181; Buchhalter J. R., Epilepsia (1993), 34, S31-S41).

An alternative assay indicative of potential anticonvulsant activity is binding to levetiracetam binding site (LBS) as hereinafter described. As set forth in U.S. patent applications Ser. Nos. 10/308,163 and 60/430,372, LBS has been identified as belonging to the family of SV2 proteins. As used herein reference to “LBS” is to be understood as including reference to SV2.

Activity in any of the above-mentioned indications can of course be determined by carrying out suitable clinical trials in a manner known to a person skilled in the relevant art for the particular indication and/or in the design of clinical trials in general.

For treating diseases, compounds of formula (I) or their pharmaceutically acceptable salts may be employed at an effective daily dosage and administered in the form of a pharmaceutical composition.

Therefore, another embodiment of the present invention concerns a pharmaceutical composition comprising an effective amount of a compound of formula (I) or a pharmaceutically acceptable salt thereof in combination with a pharmaceutically acceptable diluent or carrier.

To prepare a pharmaceutical composition according to the invention, one or more of the compounds of formula (I) or a pharmaceutically acceptable salt thereof is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral or intranasal.

Pharmaceutical compositions comprising compounds according to the invention can, for example, be administered orally, parenterally, i.e., intravenously, intramuscularly or subcutaneously, intrathecally, by inhalation or intranasally.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula (I) in compositions for oral administration is at least 0.5% by weight and can be up to 80% by weight with respect to the total weight of the composition.

In accordance with the invention it has also been found that the compounds of formula (I) or the pharmaceutically acceptable salts thereof can be administered alone or in combination with other pharmaceutically active ingredients. Non-limiting examples of such additional compounds which can be cited for use in combination with the compounds according to the invention are antivirals, antispastics (e.g. baclofen), antiemetics, antimanic mood stabilizing agents, analgesics (e.g. aspirin, ibuprofen, paracetamol), narcotic analgesics, topical anesthetics, opioid analgesics, lithium salts, antidepressants (e.g. mianserin, fluoxetine, trazodone), tricyclic antidepressants (e.g. imipramine, desipramine), anticonvulsants (e.g. valproic acid, carbamazepine, phenytoin), antipsychotics (e.g. risperidone, haloperidol), neuroleptics, benzodiazepines (e.g. diazepam, clonazepam), phenothiazines (e.g. chlorpromazine), calcium channel blockers, amphetamine, clonidine, lidocaine, mexiletine, capsaicin, caffeine, quetiapine, serotonin antagonists, β-blockers, antiarrhythmics, triptans, ergot derivatives and amantadine.

Of particular interest in accordance with the present invention are combinations of at least one compound of formula (I) or a pharmaceutically acceptable salt thereof and at least one compound inducing neural inhibition mediated by GABA_{A} receptors. The compounds of formula (I) exhibit a potentiating effect on the compounds inducing neural inhibition mediated by GABA_{A} receptors enabling, in many cases, effective treatment of conditions and disorders under reduced risk of adverse effects.

Examples of compounds inducing neural inhibition mediated by GABA_{A} receptors include the following: benzodiazepines, barbiturates, steroids, and anticonvulsants such as valproate, viagabatrine, tiagabine or pharmaceutical acceptable salts thereof.

Benzodiazepines include the 1,4-benzodiazepines, such as diazepam and clonazepam, and the 1,5-benzodiazepines, such as clobazam. Preferred compound is clonazepam.

Barbiturates include phenobarbital and pentobarbital. Preferred compound is phenobarbital.

Steroids include adrenocorticotropic hormones such as tetracosactide acetate, etc.

Anticonvulsants include hydantoins (phenytoin, ethotoin, etc), oxazolidines (trimethadione, etc.), succinimides (ethosuximide, etc.), phenacemides (phenacemide, acetylpheneturide, etc.), sulfonamides (sulthiame, acetoazolamide, etc.), aminobutyric acids (e.g. gamma-amino-beta-hydroxybutyric acid, etc.), sodium valproate and derivatives, carbamazepine and so on.

Preferred compounds include valproic acid, valpromide, valproate pivoxil, sodium valproate, semi-sodium valproate, divalproex, clonazepam, phenobarbital, vigabatrine, tiagabine, amantadine.

For the preferred oral compositions, the daily dosage is in the range 3 to 3000 milligrams (mg) of compounds of formula (I).

In compositions for parenteral administration, the quantity of compound of formula (I) present is at least 0.5% by weight and can be up to 33% by weight with respect to the total weight of the composition. For the preferred parenteral compositions, the dosage unit is in the range 3 mg to 3000 mg of compounds of formula I.

The daily dose can fall within a wide range of dosage units of compound of formula (I) and is generally in the range 3 to 3000 mg. However, it should be understood that the specific doses can be adapted to particular cases depending on the individual requirements, at the physician's discretion.

The LBS binding compounds provided by this invention and labeled derivatives thereof may be useful as standards and reagents in determining the ability of tested compounds (e.g., a potential pharmaceutical) to bind to the LBS receptor.

Labeled derivatives of LBS ligands provided by this invention may also be useful as radiotracers for positron emission tomography (PET) imaging or for single photon emission computerized tomography (SPECT).

The present invention therefore further provides labelled ligands as tools to screen chemical libraries for the discovery of potential pharmaceutical agents, in particular for treatment and prevention of the conditions set forth herein, on the basis of more potent binding to LBS/SV2 proteins, for localizing SV2 proteins in tissues, and for characterizing purified SV2 proteins. SV2 proteins include SV2A, SV2B, and SV2C whereby SV2A is the binding site for the anti-seizure drug levetiracetam and its analogs. The SV2 isoforms SV2A, SV2B, or SV2C can be derived from tissues, especially brain, from any mammal species, including human, rat or mice. Alternately the isoforms may be cloned versions of any mammalian species, including human, rat, and mice, heterologously expressed and used for assays. The screening method comprises exposing brain membranes, such as mammalian or human brain membranes, or cell lines expressing SV2 proteins or fragments thereof, especially SV2A, but including SV2B and SV2C, to a putative agent and incubating the membranes or proteins or fragments and the agent with labelled compound of formula I. The method further comprises determining if the binding of the compound of formula (I) to the protein is inhibited by the putative agent, thereby identifying binding partners for the protein. Thus, the screening assays enable the identification of new drugs or compounds that interact with LBS/SV2. The present invention also provides photoactivable ligands of SV2/LBS.

The labelled-ligands can also be used as tools to assess the conformation state of SV2 proteins after solubilization, purification and chromatography. The labelled-ligands may be directly or indirectly labeled. Examples of suitable labels include a radiolabel, such as ³H, a fluorescent label, an enzyme, europium, biotin and other conventional labels for assays of this type.

Screening assays of the present invention include methods of identifying agents or compounds that compete for binding to the LBS (especially SV2A). Labelled compounds of formula (I) are useful in the methods of the invention as probes in assays to screen for new compounds or agents that bind to the LBS (especially SV2A). In such assay embodiments, ligands can be used without modification or can be modified in a variety of ways; for example, by labelling, such as covalently or non-covalently joining a moiety which directly or indirectly provides a detectable signal. In any of these assays, the materials can be labelled either directly or indirectly. Possibilities for direct labelling include label groups such as: radiolabels including, but not limited to, [³H], [¹⁴C], [³²P], [³⁵S] or [¹²⁵I], enzymes such as peroxidase and alkaline phosphatase, and fluorescent labels capable of monitoring the change in fluorescence intensity, wavelength shift, or fluorescence polarization, including, but not limited to, fluorescein or rhodamine. Possibilities for indirect labelling include biotinylation of one constituent followed by binding to avidin coupled to one of the above label groups or the use of anti-ligand antibodies. The compounds may also include spacers or linkers in cases where the compounds are to be attached to a solid support. To identify agents or compounds which compete or interact with labelled ligands according to the invention for binding to the LBS (especially SV2A), intact cells, cellular or membrane fragments containing SV2A or the entire SV2 protein or a fragment comprising the LBS of the SV2 protein can be used. The agent or compound may be incubated with the cells, membranes, SV2 protein or fragment prior to, at the same time as, or after incubation with Levetiracetam or an analog or derivative thereof. Assays of the invention may be modified or prepared in any available format, including high-throughput screening (HTS) assays that monitor the binding of Levetiracetam or the binding of derivatives or analogs thereof to SV2 or to the LBS of the SV2 protein. In many drug screening programs which test libraries of compounds, high throughput assays are desirable in order to maximize the number of compounds surveyed in a given period of time. Such screening assays may use intact cells, cellular or membrane fragments containing SV2 as well as cell-free or membrane-free systems, such as may be derived with purified or semi-purified proteins. The advantage of the assay with membrane fragment containing SV2 or purified SV2 proteins and peptides is that the effects of cellular toxicity and/or bioavailability of the test compound can be generally ignored, the assay instead being focused primarily on the effect of the drug on the molecular target as may be manifest in an inhibition of, for instance, binding between two molecules. The assay can be formulated to detect the ability of a test agent or compound to inhibit binding of labeled ligand according to the invention to SV2 or a fragment of SV2 comprising the LBS or of Levetiracetam, or derivatives or analogs thereof, to SV2 or a fragment of SV2 comprising the LBS. The inhibition of complex formation may be detected by a variety of techniques such as filtration assays, Flashplates (Perkin Elmer, scintillation proximity assays (SPA, Amersham Biosciences). For high-throughput screenings (HTS), scintillation proximity assay is a powerful method which uses microspheres coated with biological membranes and requires no separation or washing steps.

Labelled ligands are also useful for assessing the conformational state of SV2 after solubilization, purification, and chromatography. Moreover, the present invention provides photoactivable versions of the ligands for labelling and detection in biological samples. The photoactivable ligands may also be used to localize and purify SV2 from tissues, isolated cells, subcellular fractions and membranes. The photoactivable could also be used for SV2 cross-linking and identification of binding domains of LBS ligands.

### EXAMPLES

The following examples are provided for illustrative purposes. Unless specified otherwise in the examples, characterization of the compounds is performed according to the following methods:

NMR spectra are recorded on a BRUKER AC 250 Fourier Transform NMR Spectrometer fitted with an Aspect 3000 computer and a 5 mm ¹H/¹³C dual probehead or BRUKER DRX 400 FT NMR fitted with a SG Indigo² computer and a 5 mm inverse geometry ¹H/¹³C/¹⁵N triple probehead. The compound is studied in DMSO-d₆ (or CDCl₃) solution at a probe temperature of 313 K or 300 K and at a concentration of 20 mg/ml. The instrument is locked on the deuterium signal of DMSO-d₆ (or CDCl₃). Chemical shifts are given in ppm downfield from TMS taken as internal standard.

HPLC analyses are performed using one of the following systems:

an Agilent 1100 series HPLC system mounted with an INERTSIL ODS 3 C18, DP 5 μm, 250×4.6 mm column. The gradient ran from 100% solvent A (acetonitrile, water, H₃PO₄ (5/95/0.001, v/v/v)) to 100% solvent B (acetonitrile, water, H₃PO₄ (95/5/0.001, v/v/v)) in 6 min with a hold at 100% B of 4 min. The flow rate is set at 2.5 ml/min. The chromatography is carried out at 35° C.

a HP 1090 series HPLC system mounted with a HPLC Waters Symmetry C18, 250×4.6 mm column. The gradient ran from 100% solvent A (MeOH, water, H₃PO₄ (15/85/0.001M, v/v/M)) to 100% solvent B (MeOH, water, H₃PO₄ (85/15/0.001 M, v/v/M)) in 10 min with a hold at 100% B of 10 min. The flow rate is set at 1 ml/min. The chromatography is carried out at 40° C.

Mass Spectrometric Measurements in LC/MS mode are Performed as Follows:

HPLC Conditions

Analyses are performed using a WATERS Alliance HPLC system mounted with an INERTSIL ODS 3, DP 5 μm, 250×4.6 mm column.

The gradient ran from 100% solvent A (acetonitrile, water, TFA (10/90/0.1, v/v/v)) to 100% solvent B (acetonitrile, water, TFA (90/10/0.1, v/v/v)) in 7 min with a hold at 100% B of 4 min. The flow rate is set at 2.5 ml/min and a split of 1/25 is used just before API source.

MS Conditions

Samples are dissolved in acetonitrile/water, 70/30, v/v at the concentration of about 250 μgr/ml. API spectra (+ or −) are performed using a FINNIGAN (San Jose, Calif., USA) LCQ ion trap mass spectrometer. APCI source operated at 450° C. and the capillary heater at 160° C. ESI source operated at 3.5 kV and the capillary heater at 210° C.

Mass spectrometric measurements in DIP/EI mode are performed as follows: samples are vaporized by heating the probe from 50° C. to 250° C. in 5 min. EI (Electron Impact) spectra are recorded using a FINNIGAN (San Jose, Calif., USA) TSQ 700 tandem quadrupole mass spectrometer. The source temperature is set at 150° C.

Mass spectrometric measurements on a TSQ 700 tandem quadrupole mass spectrometer (Finnigan MAT, San Jose, Calif., USA) in GC/MS mode are performed with a gas chromatograph model 3400 (Varian, Walnut Creek, Calif., USA) fitted with a split/splitless injector and a DB-5MS fused-silica column (15 m×0.25 mm I.D., 1 μm) from J&W Scientific (Folsom, Calif., USA). Helium (purity 99.999%) is used as carrier gas. The injector (CTC A200S autosampler) and the transfer line operate at 290 and 250° C., respectively. Sample (1 μl) is injected in splitless mode and the oven temperature is programmed as follows: 50° C. for 5 min., increasing to 280° C. (23° C./min) and holding for 10 min. The TSQ 700 spectrometer operates in electron impact (EI) or chemical ionization (Cl/CH₄) mode (mass range 33-800, scan time 1.00 sec). The source temperature is set at 150° C. Also used is a 1100 LCMSD VL series, single quadrupole, APCI or API-ES ionization (Agilent Technologies, USA) equipped with the following HPLC columns: Luna C18 5 um 100×4.6 mm (Phenomenex, USA) or Hi-Q C18 5 um 100×4.6 mm (Peeke Scientific, USA) or Betasil C18 10 um 150×4.6 mm (ThermoHypersil, USA). GC/MS are also done with GC 6890 equipped with FID and 5973 MSD, single quadrupole, EI ionization (Agilent Technologies, USA) equipped with column: HP-5MS 30 m×0.25 mm×0.25 um (Agilent Technologies, USA).

Specific rotation is recorded on a Perkin-Elmer 341 polarimeter. The angle of rotation is recorded at 25° C. on 1% solutions in MeOH. For some molecules, the solvent is CH₂Cl₂ or DMSO, due to solubility problems.

Melting points are determined on a Büchi 535 or 545 Tottoli-type fusionometre, and are not corrected, or by the onset temperature on a Perkin Elmer DSC 7.

Preparative chromatographic separations are performed on silicagel 60 Merck, particle size 15-40 μm, reference 1.15111.9025, using Novasep axial compression columns (80 mm i.d.), flow rates between 70 and 150 ml/min. Amount of silicagel and solvent mixtures as described in individual procedures.

Preparative Chiral Chromatographic separations are performed on a DAICEL Chiralpak AD 20 μm, 100*500 mm column using an in-house build instrument with various mixtures of lower alcohols and C5 to C8 linear, branched or cyclic alkanes at ±350 ml/min. Solvent mixtures as described in individual procedures.

The following abbreviations are used in the examples:

AcOEt Ethyl acetate

CH₃CN Acetonitrile

CH₂Cl₂ Dichloromethane

DCE 1,2-dichloroethane

DMF N,N-Dimethylformamide

MTBE Methyl tert-butyl ether

NBS N-bromosuccinimide

NCS N-chlorosuccinimide

NIS N-iodosuccinimide

TFA Trifluoroacetic acid

THF Tetrahydrofuran

The following examples illustrate how the compounds covered by formula (I) may be obtained.

### Example 1

### Synthesis of (2S)-2-[3-(4-nitrophenyl)-5-oxoimidazolidin-1-yl]butanamide 1 and (2S)-2-(5-oxo-3-phenylimidazolidin-1-yl)butanamide 3

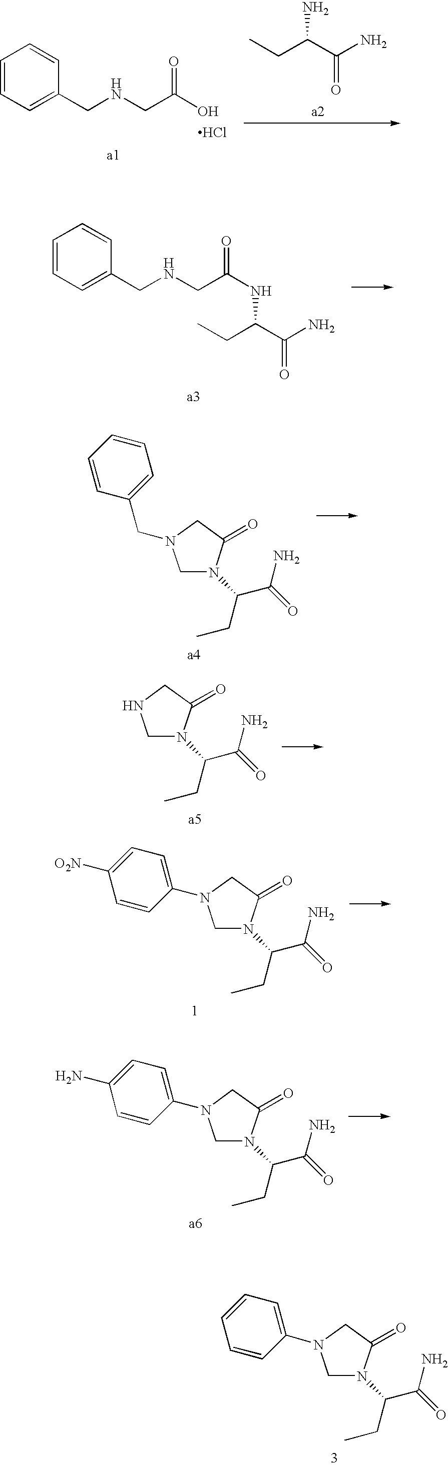

### 1.1 Synthesis of (2S)-2-{[(benzylamino)acetyl]amino}butanamide a3

In a three necked flask fitted with a magnetic stirrer and a reflux condenser, under inert atmosphere, triethylamine (69 mL, 496 mmol) is added dropwise to a solution of (benzylamino)acetic acid a1 (50 g, 248 mmol) in a mixture of DMF (400 mL) and CHCl₃ (400 mL) at 0° C. The mixture is stirred at 0° C. for 10 minutes and at room temperature for 15 minutes. 1H-1,2,3-benzotriazol-1-ol (36.9 g, 273 mmol), (2S)-2-aminobutanamide a2 (30.4 g, 298 mmol) and N,N′-dicyclohexylcarbodiimide (56.33 g, 273 mmol) are respectively added to the mixture, each one of them in solution of 250 mL (or 300 mL) of CHCl₃/DMF (50/50 v/v). DMF (200 mL) is added and the mixture is heated at 45° C. for 4 h, and stirred at room temperature for 3 days. The mixture is partially concentrated (⅔ of solvent is removed) and again heated at 45° C. overnight. The whole solvent is removed under reduced pressure, the residue is dissolved in CH₂Cl₂ and filtered. The organic phase is concentrated, the obtained residue is purified by chromatography on silicagel (CH₂Cl₂/C₂H₅OH/NH₄OH 94/6/0.6 v/v/v) and recrystallized from AcOEt to afford (2S)-2-{[(benzylamino)acetyl]amino}butanamide a3 (36.8 g). Yield: 58.9%.

¹H NMR δ_{H} (250 MHz, DMSO, ppm): 0.85 (t, 3H), 1.5-1.8 (m, 2H), 2.7 (s, 1H), 3.15 (s, 2H), 3.7 (s, 2H), 4.25 (m, 1H), 7.0 (s, 1H), 7.2-7.5 (m, 6H), 7.9 (d, 1H).

### 1.2 Synthesis of (2S)-2-(3-benzyl-5-oxoimidazolidin-1-yl)butanamide a4

In a three necked flask fitted with a magnetic stirrer and a reflux condenser, a mixture of (2S)-2-{[(benzylamino)acetyl]amino}butanamide a3 (36.8 g, 148 mmol) and formaldehyde (91 mL, 37% in water) is heated at 78° C. for 1 hour and cooled down to room temperature. The mixture is extracted by AcOEt (3×300 mL), the combined organic phases are dried over MgSO₄ and concentrated under reduced pressure. The residue is purified twice by chromatography on silicagel (eluent 1: CH₂Cl₂/C₂H₅OH/NH₄OH 94/6/0.6 v/v/v; eluent 2: CH₂Cl₂/isopropanol/NH₄OH 95/5/0.5 v/v/v) to afford (2S)-2-(3-benzyl-5-oxoimidazolidin-1-yl)butanamide a4 (26.3 g). Yield: 68%. GC-MS (M⁺·): 261.

### 1.3 Synthesis of (2S)-2-(5-oxoimidazolidin-1-yl)butanamide a5

In a 2 L pressured jar, under inert atmosphere, (2S)-2-(3-benzyl-5-oxo-1-imidazolidinyl)-butanamide a4 (24.4 g, 93 mmol) is dissolved in ethanol (1 L). Pd/C (10%, 6.9 g, 30% wt) is added and the mixture is hydrogenated on a Parr hydrogenator for 2.5 hours. The mixture is degassed, filtered on celite and the filtrate is concentrated under reduced pressure. The crude residue is purified by chromatography on silicagel (CH₂Cl₂/CH₃OH/NH₄OH 88/12/1.2 v/v/v) to afford 15.48 g of (2S)-2-(5-oxoimidazolidin-1-yl)butanamide a5.

Yield: 97%.

¹H NMR δ_{H} (250 MHz, DMSO+D₂O, ppm): 0.85 (t, 3H), 1.5-1.9 (m, 2H), 3.3 (s, 2H), 4.25 (m, 2H), 4.45 (m, 1H), 6.9-7.4 (2 s (broad), 2×1H).

### 1.4 Synthesis of (2S)-2-[3-(4-nitrophenyl)-5-oxoimidazolidin-1-yl]butanamide 1

In a three necked flask fitted with a magnetic stirrer and a reflux condenser, (2S)-2-(5-oxoimidazolidin-1-yl)butanamide a5 (5 g, 29 mmol) and K₂CO₃ (4.04 g, 29 mmol) are dissolved in DMSO (25 mL). A solution of 1-fluoro-4-nitrobenzene (4.2 g, 29 mmol) in DMSO (15 mL) is added dropwise and the mixture is heated at 60° C. overnight. The mixture is cooled to room temperature, K₂CO₃ (0.81 g, 6 mmol) and 1-fluoro-4-nitrobenzene (0.84 g, 6 mmol, in 0.6 mL of DMSO) are added. The mixture is heated again at 60° C. for 3 hours, at 70° C. for 75 minutes and concentrated under reduced pressure. The crude reaction mixture is purified by chromatography on silicagel (CH₂Cl₂/CH₃OH/NH₄OH 88/12/1.2 v/v/v) to afford 2.46 g of (2S)-2-[3-(4-nitrophenyl)-5-oxoimidazolidin-1-yl]butanamide 1. Yield: 29%. GC-MS (M⁺·): 292.

### 1.5 Synthesis of (2S)-2-[3-(4-aminophenyl)-5-oxoimidazolidin-1-yl]butanamide a6

In a Parr pressure jar, under inert atmosphere, (2S)-2-[3-(4-nitrophenyl)-5-oxoimidazolidin-1-yl]butanamide 1 (1.79 g, 6.1 mmol) is dissolved in a mixture of methanol (180 mL) and CHCl₃ (10 mL). 10% Pd on charcoal (1 g, 55% wt) is added and the mixture is hydrogenated under 40 psi for 2 hours. The mixture is degassed and filtered on celite. The filtrate containing (2S)-2-[3-(4-aminophenyl)-5-oxoimidazolidin-1-yl]butanamide a6 is directly used in the next step.

### 1.6 Synthesis of (2S)-2-(5-oxo-3-phenylimidazolidin-1-yl)butanamide 3

In a three necked flask fitted with a magnetic stirrer and a reflux condenser, under inert atmosphere, the solution of (2S)-2-[3-(4-aminophenyl)-5-oxoimidazolidin-1-yl]butanamide a6 in a mixture of methanol/CHCl₃ (95/5 v/v) obtained in step 1 (200 mL, 4.88 mmol) is cooled to 0° C. Concentrated H₂SO₄ (6 mL, 115 mmol) is added and NaNO₂ (0.65 g, 9.76 mmol) is added carefully. The mixture is stirred at room temperature for 1 hour and a solution of sodium hypophosphite monohydrate (10.66 g, 97.6 mmol) in 80 mL of water is dropwise added at 14° C. After 10 minutes at 18° C., CaSO₄ is added and the mixture is stirred at room temperature for 2 hours. The mixture is poured into 300 mL of cold water, basified to pH 8.5 by the addition of NaOH 10% (w/w), and extracted 3 times with AcOEt. The combined extracts are dried over MgSO₄ and concentrated under reduced pressure to give after purification by chromatography on silicagel (CH₂Cl₂/i-PrOH/NH₄OH 97/3/0.3 v/v/v) and recrystallization from EtOAc, (2S)-2-(5-oxo-3-phenylimidazolidin-1-yl)butanamide 3 (0.365 g). Yield: 30%. MS (dip-MS, M⁺·): 247.

### Example 2

### Synthesis of (2S)-2-[3-(2,4-dinitrophenyl)-5-oxoimidazolidin-1-yl]butanamide 2

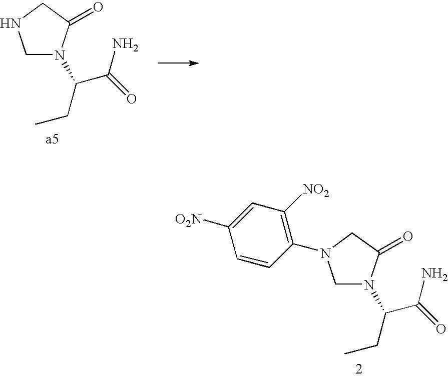

In a three necked flask fitted with a magnetic stirrer and a reflux condenser, under inert atmosphere, (2S)-2-(5-oxoimidazolidin-1-yl)butanamide a5 (1 g, 5.84 mmol) is dissolved in ethanol (10 mL). K₂CO₃ (0.81 g, 5.84 mmol) is added and the mixture is cooled down to 0° C. A solution of 1-fluoro-2,4-dinitrobenzene (1.08 g, 5.84 mmol) in ethanol (2 mL) is added dropwise and the mixture is stirred overnight at room temperature. The solvent is removed under reduced pressure, the residue is purified by chromatography on silicagel (CH₂Cl₂/CH₃OH/NH₄OH 96/4/0.4 v/v/v) and recrystallized from acetonitrile to give (2S)-2-[3-(2,4-dinitrophenyl)-5-oxoimidazolidin-1-yl]butanamide 2 (1.148 g).

Yield: 58%. MS (dip-MS, MH⁺): 338.

### Example 3

### Synthesis of (2S)-2-[5-(iodomethyl)-2-oxo-1,3-oxazolidin-3-yl]butanamide 4

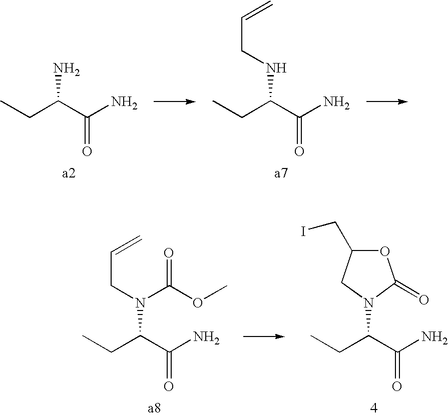

### 3.1 Synthesis of (2S)-2-(allylamino)butanamide a7

In a three necked flask fitted with a magnetic stirrer, under inert atmosphere, (2S)-2-aminobutanamide a2 (30 g, 290 mmol) is dissolved in DMF (300 mL) and K₂CO₃ (4 g, 29 mmol) is added. The mixture is cooled to 0° C. and allyl bromide (2.5 mL, 29 mmol) is added. After 1 h at 0° C., the mixture is concentrated and the residue is purified by chromatography on silicagel (CH₂Cl₂/EtOH/NH₄OH 97/2.7/0.3 v/v/v) to give (2S)-2-(allylamino)butanamide a7 (4.1 g). Yield: 100%. LC-MS (MH⁺): 143.

### 3.2 Synthesis of methyl allyl[1-(aminocarbonyl)propyl]carbamate a8

In a three necked flask fitted with a magnetic stirrer, under inert atmosphere, 2-(allylamino)butanamide a7 (3.73 g, 26.3 mmol) is dissolved in CH₂Cl₂ (40 mL). K₂CO₃ (4 g, 289.3 mmol) is added to the solution. The mixture is cooled to 0° C. for the addition of methyl chloroformate (2.25 mL, 28.9 mmol). After stirring overnight at room temperature, the solvent is removed under reduced pressure and the residue is purified by chromatography on silicagel (CH₂Cl₂/C₂H₅OH 95/5 v/v) to afford methyl allyl[1-(aminocarbonyl)propyl]carbamate a8 (4.93 g). Yield: 94%. GC-MS (M⁺·): 200.

### 3.3 Synthesis of (2S)-2-[5-(iodomethyl)-2-oxo-1,3-oxazolidin-3-yl]butanamide 4

In a three necked flask fitted with a magnetic stirrer, under inert atmosphere, methyl allyl[1-(aminocarbonyl)propyl]carbamate a8 (4.69 g, 23.45 mmol) is dissolved in CH₂Cl₂ (55 mL), KI (5.84 g, 35.18 mmol) and 1₂ (17.87 g, 70.35 mmol) are added to the mixture. After stirring at room temperature for 2 hours, the mixture is concentrated and purified by chromatography on silicagel (the crude solid is set down on the silicagel without dilution; tert-butyl methyl ether/2-propanol 95/5 v/v), recrystallized from toluene and ground up to obtain (2S)-2-[5-(iodomethyl)-2-oxo-1,3-oxazolidin-3-yl]butanamide 4 (0.7 g).

Yield: 9%. LC-MS (MH⁺): 313.

### Example 4

### Synthesis of (2S)-2-(2-oxo-2,5-dihydro-1H-pyrrol-1-yl)butanamide 5

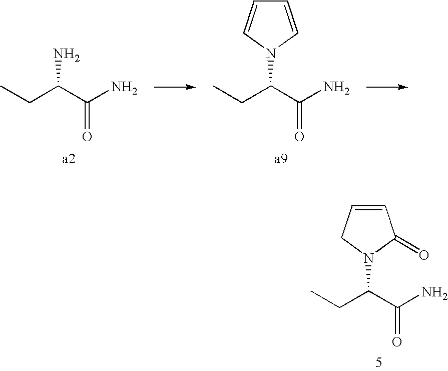

### 4.1 Synthesis of (2S)-2-(1H-pyrrol-1-yl)butanamide a9

In a three necked flask fitted with a magnetic stirrer and a reflux condenser, under inert atmosphere, (2S)-2-aminobutanamide a2 (20.4 g, 190 mmol) is dissolved in 250 mL of acetic acid, 2,5-dimethoxytetrahydrofuran (25.5 g, 190 mmol) is added and the mixture is brought to reflux for 45 minutes. The solvent is removed under reduced pressure and the residue is purified by chromatography on silicagel (CH₂Cl₂/i-PrOH 98/2 v/v) to give (2S)-2-(1H-pyrrol-1-yl)butanamide a9 (13.76 g). Yield: 48%. GC-MS (M⁺·): 152.

### 4.2 Synthesis of (2S)-2-(2-oxo-2,5-dihydro-1H-pyrrol-1-yl)butanamide 5

In a three necked flask fitted with a magnetic stirrer and a reflux condenser, under inert atmosphere, (2S)-2-(1H-pyrrol-1-yl)butanamide a9 (8.61 g, 56.6 mmol) is dissolved in CHCl₃ (150 mL). K₂CO₃ (9.39 g, 67.9 mmol) is added to the mixture, and a solution of 4-chloroperbenzoic acid (mCPBA, 25.5 g, 67.9 mmol) in CHCl₃ (250 mL) is added dropwise over 1.5 hours. The mixture is stirred at room temperature for 6 hours. A solution of mCPBA (10.6 g, 28.2 mmol) in 100 mL of CHCl₃ is added twice (after 2.5 h and after 5 h). The mixture is filtered and the precipitate is washed with CHCl₃. The filtrate is concentrated under reduced pressure. The residue is purified by chromatography on silicagel (CH₂Cl₂/C₂H₅OH 95/5 v/v), by preparative chiral chromatography (column: Chiralpak AD 250*4.6 mm; CH₃OH/EtOH/isohexane 12/3/85 v/v/v) and recrystallized from AcOEt to afford 0.302 g of (2S)2-(2-oxo-2,5-dihydro-1H-pyrrol-1-yl)butanamide 5.

Yield: 3%. GC-MS (M⁺·): 168.

### Example 5

### Synthesis of 2-(2-oxo-4-phenyl-2,5-dihydro-1H-pyrrol-1-yl)butanamide 6

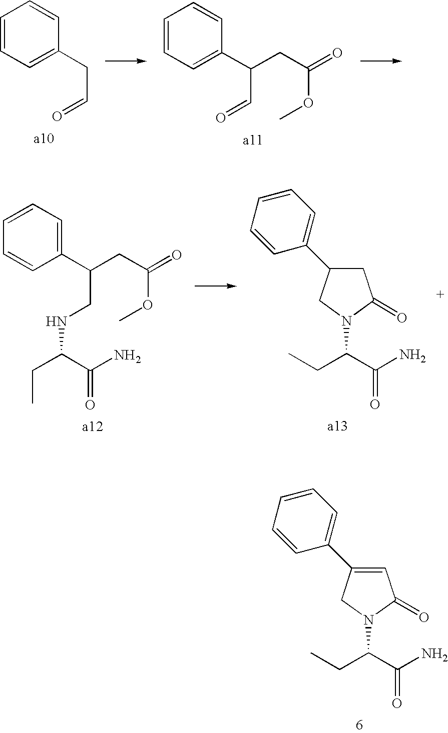

### 5.1 Synthesis of methyl 4-oxo-3-phenylbutanoate a11

In a 3 L three-necked flask fitted with a magnetic stirrer, a Dean-Stark apparatus and a reflux condenser, phenylacetaldehyde a10 (150 mL, 1150 mmol), toluene (750 mL) and diisobutylamine (202 mL, 1150 mmol) are heated at reflux for 2.5 h under inert atmosphere. The mixture is concentrated to dryness and redissolved in acetonitrile (300 mL). Methylbromoacetate (106 mL, 1150 mmol) is added and the solution is heated at reflux for 4 h. At room temperature, acetic acid (70 mL) and water (210 mL) are added and the mixture is stirred for 0.5 h at room temperature and for 1 h at 45° C. Acetonitrile is removed under reduced pressure and the mixture is extracted with CH₂Cl₂. The organic phase is washed with a saturated solution of NaHCO₃, dried over MgSO₄ and concentrated in vacuo. The crude mixture is purified by distillation under reduced pressure (110° C., 0.5 mmHg) to afford methyl 4-oxo-3-phenylbutanoate a11 (151.5 g) as a yellow liquid.

Yield: 69%. LC-MS (MH⁺): 193.

### 5.2 Synthesis of methyl 4-{[(1S)-1-(aminocarbonyl)propyl]amino}-3-phenylbutanoate a12

In a 3 L three necked flask fitted with a mechanical stirrer and a reflux condenser, under inert atmosphere, methyl 4-oxo-3-phenylbutanoate a11 (151.2 g, 790 mmol) is dissolved in MeOH (1200 mL). Molecular sieves (3 A, 24 g) are added, followed by (2S)-2-aminobutanamide (80.3 g, 790 mmol). The mixture is heated at 65° C. for 1 h, cooled down to room temperature and filtered. NaBH₃CN (52.2 g, 790 mmol) is added, the temperature raises to 45° C. and the mixture is stirred at room temperature for 2 h. A saturated solution of NH₄Cl is added (1 L), the mixture is extracted with CH₂Cl₂, the organic phase is dried over MgSO₄ and concentrated to dryness under reduce pressure. The crude reaction mixture is purified twice by preparative chromatography on silicagel (eluent 1: CH₂Cl₂/MeOH/NH₄OH 95/4.5/0.5 v/v/v; eluent 2: CH₂Cl₂/hexane 50/50 v/v) to afford methyl 4-{[(1S)-1-(aminocarbonyl)propyl]amino}-3-phenylbutanoate a12 (101 g).Yield: 52%.

### 5.3 Synthesis of 2-(2-oxo-4-phenyl-2,5-dihydro-1H-pyrrol-1-yl)butanamide 6

Methyl 4-{[(1S)-1-(aminocarbonyl)propyl]amino}-3-phenylbutanoate a12 is dissolved in acetic acid/MeOH (1/1 v/v) and heated at 80° C. for 2 h, cooled down to room temperature and concentrated under reduce pressure. The obtained 1/1 diastereomeric mixture is purified by chromatography on silicagel (CH₂Cl₂/i-PrOH 95/5 v/v) and by chiral chromatography (column: Chiralpack AD 250*4.6 mm; eluent: EtOH/diethylamine 100/0.1 v/v) to afford (2S)-2-(2-oxo-4-phenyl-1-pyrrolidinyl)butanamide a13 (20.8 g), and 2-(2-oxo-4-phenyl-2,5-dihydro-1H-pyrrol-1-yl)butanamide 6 (1.19 g) as secondary product. This compound is recrystallized from toluene to give pure (2S)-2-(2-oxo-4-phenyl-2,5-dihydro-1H-pyrrol-1-yl)butanamide 6 (0.87 g). Yield: 0.5%. LC-MS (MH⁺): 245.

### Example 6

### Synthesis of (2S)-2-(4-methyl-2-oxo-2,5-dihydro-1H-pyrrol-1-yl)butanamide 7

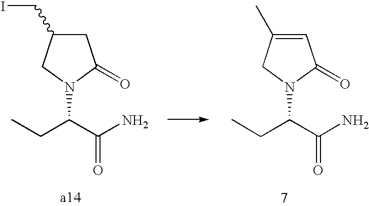

In a three necked flask fitted with a magnetic stirrer and a reflux condenser, under inert atmosphere, (2S)-2-[4-(iodomethyl)-2-oxopyrrolidin-1-yl]butanamide a14 is dissolved in DMF (50 mL) and 2,3,4,6,7,8,9,10-octahydropyrimido[1,2-a]azepine (DBU, 1.9 mL, 12.65 mmol) is added. The mixture is heated at 90° C. for 4 hours, cooled down to room temperature and concentrated. The crude residue is purified twice by chromatography on silicagel (eluent 1: gradient CH₂Cl₂/C₂H₅OH from 96/4 to 90/10 v/v; eluent 2: gradient AcOEt/C₂H₅OH from 96/4 to 90/10 v/v) to afford 0.260 g of (2S)-2-(4-methyl-2-oxo-2,5-dihydro-1H-pyrrol-1-yl)butanamide 7. Yield: 12%. LC-MS (MH⁺): 191.

### Example 7

### Synthesis of (+)-(2S)-2-(2-oxo-4-propyl-2,5-dihydro-1H-pyrrol-1-yl)butanamide 8

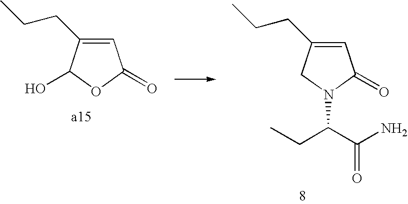

In a three neck flask, under argon, 5-hydroxy-4-propylfuran-2(5H)-one a15 (35.5 g, 0.25 mol) is added to a solution of (2S)-2-aminobutanamide a2 (28.1 g, 0.275 mol) in toluene (355 mL) at 18° C. The solution is stirred for 0.5 h at this temperature and a precipitate appears. The reaction mixture is stirred for 2 h and 4 N NaOH (37.5 mL) is added dropwise to the suspension followed by an aqueous solution of NaBH₄ (6.2 g, 0.16 mol) in water (62 mL). After 1 h, the reaction mixture is carefully quenched with AcOH (30 mL), heated to 50° C. for 3 h and cooled down to room temperature overnight. NaOH is added (20 mL, 50% w/w) and the aqueous phase is extracted with toluene (2 times). The organic phases are combined, washed with brine and concentrated under reduced pressure to afford the crude unsaturated pyrrolidone (43.4 g) as an orange oil. It is recrystallized from diisopropyl ether to afford (+)-(2S)-2-(2-oxo-4-propyl-2,5-dihydro-1H-pyrrol-1-yl)butanamide 8 as a white solid (1.8 g). Yield: 3.4%. LC-MS (MH⁺): 211. Alpha_{D}: +13.26.

### Example 8

### Synthesis of (2S)-2-(2-oxo-5-propyl-1,3-thiazol-3(2H)-yl)butanamide 9

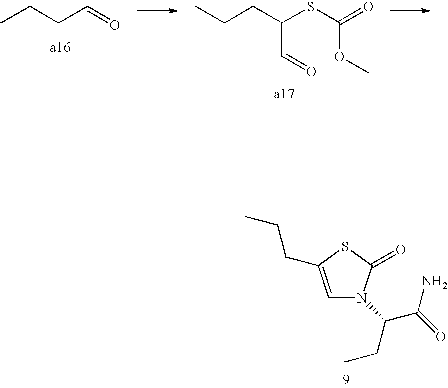

### 8.1 Synthesis of S-(1-formylbutyl)O-methyl thiocarbonate a17

In a three necked flask fitted with a magnetic stirrer, under inert atmosphere, butyraldehyde a16 (1.89 g, 22 mmol) is dissolved in CHCl₃ (20 mL).

Methoxycarbonylsulfenyl chloride (2 mL, 22 mmol) is added and the reaction mixture is stirred at room temperature overnight. The mixture is concentrated and the residue is purified by chromatography on silicagel (AcOEt/hexane 10:90) to afford 2.63 g of S-(1-formylbutyl)-O-methyl thiocarbonate a17.

Yield: 68%. LC-MS (MH⁺): 177.

### 8.2 Synthesis of (2S)-2-(2-oxo-5-propyl-1,3-thiazol-3(2H)-yl)butanamide 9

In a three necked flask fitted with a magnetic stirrer, under inert atmosphere, S-(1-formylbutyl)-O-methyl thiocarbonate a17 (0.5 g, 2.8 mmol) and (2S)-2-aminobutanamide a2 (0.290 g, 2.8 mmol) are dissolved in toluene (5 mL). p-toluenesulfonyl chloride (catalytic amount) is added and the mixture is heated to reflux for 1,5 hours. Acetic acid (1 mL) is added to the hot solution and the mixture is concentrated under reduced pressure. The residue is dissolved in AcOEt, washed with water and a saturated solution of NaHCO₃ The organic layer is dried over MgSO₄, filtered and concentrated under reduced pressure. The crude reaction mixture is purified by chromatography on silicagel (CH₂Cl₂/CH₃OH/NH₄OH 97/2.7/0.3 v/v/v). Crude 9 is triturated in Et₂O to afford 0.4 g of pure (2S)-2-(2-oxo-5-propyl-1,3-thiazol-3(2H)-yl)butanamide 9. Yield: 63%. LC-MS (MH⁺): 229.

### Example 9

### Synthesis of 2-(2-oxo-5-propyl-1,3-thiazol-3(2H)-yl)propanamide 10

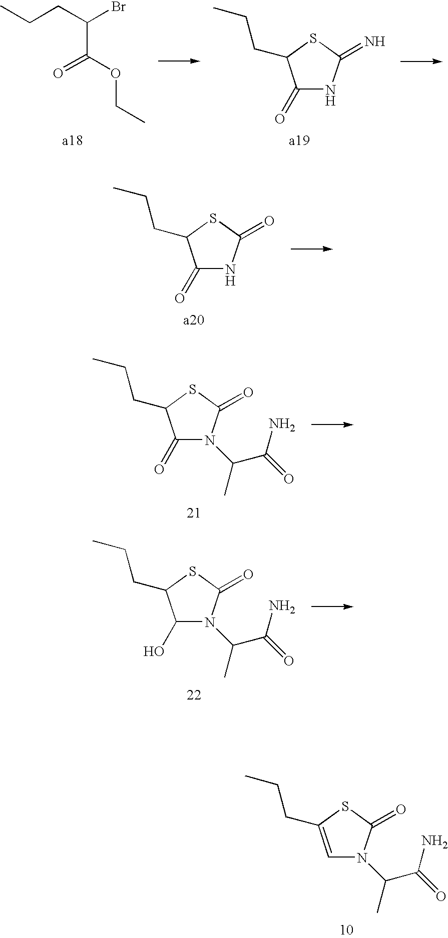

### 9.1 Synthesis of 2-amino-5-propyl-1,3-thiazol-4(5H)-one a19

In a three necked flask fitted with a magnetic stirrer and a reflux condenser, under nitrogen atmosphere, ethyl 2-bromopentanoate a18 (8.36 g, 40 mmol), thiourea (3.06 g, 40 mmol) and sodium acetate (3.30 g, 40 mmol) are dissolved in 160 mL of ethanol. The mixture is heated at 65° C. for 2.5 hours. The reaction mixture is cooled down to room temperature and partially concentrated under reduced pressure. A saturated solution of NaHCO₃ is added to the white precipitate till neutral pH (after the addition, the precipitate dissolves and precipitates again). Et₂O (65 mL) and hexane (65 mL) are added, the mixture is stirred at room temperature for 0.5 h and the solid is filtered to afford 2-amino-5-propyl-1,3-thiazol-4(5H)-one a19 (5.37 g) as a white solid. Yield: 85%. LC-MS (MH⁺): 159.

### 9.2 Synthesis of 5-propyl-1,3-thiazolidine-2,4-dione a20

In a 500 mL flask, fitted with a magnetic stirrer and a reflux condenser, 2-amino-5-propyl-1,3-thiazol-4(5H)-one a19 (5.37 g, 34 mmol) is dissolved in 90 mL of HCl (2.5 N) and 90 mL of ethanol, and the mixture is heated at 65° C. for 2.5 hours. Several portions of 5 N HCl (5 mL) are added until complete consumption of starting thiazolidinone. The reaction is cooled down to room temperature. Saturated NaHCO₃ is added until neutral pH and the mixture is extracted with chloroform (3 times). The combined organic extracts are dried over anhydrous MgSO₄ and concentrated under reduced pressure to afford a first fraction of a20 (1.95 g) as an oil. The remaining aqueous phase is partially concentrated, extracted with AcOEt (3 times), the combined organic extracts are dried over MgSO₄ and concentrated under reduced pressure to afford a second batch of 5-propyl-1,3-thiazolidine-2,4-dione a20 (0.700 g) as a colorless oil. Total yield: 49%. LC-MS (MH⁻): 158.

### 9.3 Synthesis of 2-(2,4-dioxo-5-propyl-1,3-thiazolidin-3-yl)propanamide a21

In a flask fitted with a magnetic stirrer and a reflux condenser, under nitrogen atmosphere, a mixture of 5-propyl-1,3-thiazolidine-2,4-dione a20 (1.80 g, 11 mmol), K₂CO₃ (1.545 g, 12.1 mmol), 2-bromopropionamide (1.698 g, 13.2 mmol) and DMF (36 mL) is heated to 35° C. until disappearance of starting material (6 days). Cold water is added and the aqueous phase is extracted with AcOEt (3 times). The combined organic extracts are washed with brine, dried over anhydrous MgSO₄ and concentrated under reduced pressure to give 2-(2,4-dioxo-5-propyl-1,3-thiazolidin-3-yl)propanamide a21 as a yellow oil which is used in the next step without further purification. LC-MS (MH⁺): 231.

### 9.4 Synthesis of 2-(4-hydroxy-2-oxo-5-propyl-1,3-thiazolidin-3-yl)propanamide a22

In a flask fitted with a magnetic stirrer, under nitrogen atmosphere, 2-(2,4-dioxo-5-propyl-1,3-thiazolidin-3-yl)propanamide a21 is dissolved in methanol (9 mL). At 0° C., NaBH₄ (0.653 g, 22 mmol) is carefully added. The mixture is stirred at 0° C. for 25 minutes and poured into cold water. The aqueous phase is extracted with AcOEt (2×40 mL), the combined organic extracts are dried over MgSO₄ and concentrated under reduced pressure to give crude 2-(4-hydroxy-2-oxo-5-propyl-1,3-thiazolidin-3-yl)propanamide a22 which is used in the next step without further purification. LC-MS (basic, MH⁺): 215 (2 diastereoisomers).

### 9.5 Synthesis of 2-(2-oxo-5-propyl-1,3-thiazol-3(2H)-yl)propanamide 10

In a one necked flask fitted with a magnetic stirrer and a reflux condenser, 2-(4-hydroxy-2-oxo-5-propyl-1,3-thiazolidin-3-yl)propanamide a22 is dissolved in acetic acid (22 mL) and the solution refluxed (80° C.) for 3 hours. The mixture is left overnight at room temperature. Water is added and acetic acid is removed in vacuo. The mixture is neutralized by the addition of saturated NaHCO₃ and extracted with AcOEt (3×80 mL). The combined organic extracts are dried over MgSO₄ and concentrated to give the crude product which is purified by recrystallization from water to afford 2-(2-oxo-5-propyl-1,3-thiazol-3(2H)-yl)propanamide 10 (0.257 g) as a white solid. Yield (3 steps): 14%. LC-MS (MH⁺): 215.

### Example 10

### Synthesis of 2-(2-oxo-5-phenyl-1,3-thiazolidin-3-yl)butanamide 15

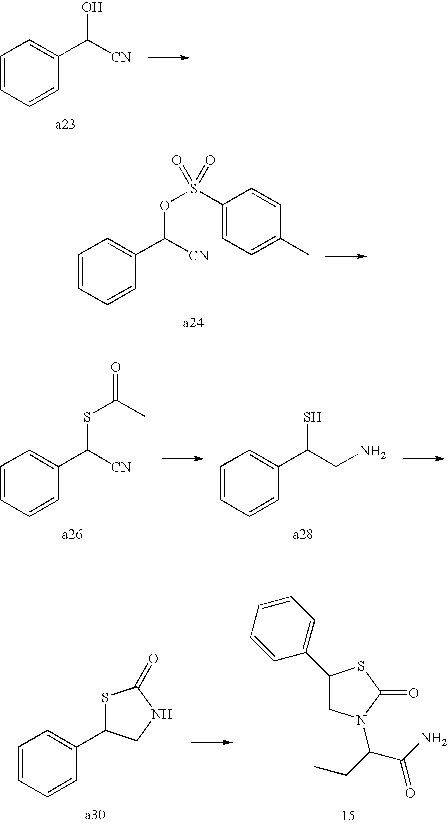

### 10.1 Synthesis of cyano(phenyl)methyl 4-methylbenzenesulfonate a24

In a three necked flask fitted with a magnetic stirrer and a dropping funnel, under inert atmosphere, hydroxy(phenyl)acetonitrile a23 (2.4 mL, 20 mmol) is dissolved in 40 mL of CH₂Cl₂. The mixture is cooled down to −10° C. and triethylamine (5.87 mL, 40 mmol) is added. A solution of p-toluenesulfonyl chloride (4.578 g, 24 mmol) in CH₂Cl₂ (100 mL) is added dropwise while maintaining the temperature under 0° C. The mixture is stirred at 0° C. for 30 minutes and Et₂O is added. The salts are filtered and the filtrate is concentrated under reduced pressure to afford crude cyano(phenyl)methyl 4-methylbenzenesulfonate a24 which is directly used in the next step.

1-cyanopentyl 4-methylbenzenesulfonate a25 may be synthesized according to the same method.

### 10.2 Synthesis of S-[cyano(phenyl)methyl]ethanethioate a26

In a three necked flask fitted with a magnetic stirrer and a dropping funnel, under inert atmosphere, cyano(phenyl)methyl 4-methylbenzenesulfonate a24 is dissolved in DMF (20 mL). A solution of potassium thioacetate (2.512 g, 22 mmol) in DMF (20 mL) is added dropwise at room temperature. After 3 h, the mixture is poured into cold water and extracted with Et₂O (2×50 mL). The combined organic extracts are washed with brine, dried over anhydrous MgSO₄ and concentrated under reduced pressure. The crude reaction mixture is purified by chromatography on silicagel (AcOEt/hexane 25/75 v/v) to afford 2.9 g of S-[cyano(phenyl)methyl]ethanethioate a26 as a yellow solid.

Yield (from a23): 76%. GC-MS (M⁺·): 191.

S-(1-cyanopentyl)ethanethioate a27 (LC-MS (MH⁺): 172) may be synthesized according to the same method.

### 10.3 Synthesis of 2-amino-1-phenylethanethiol a28

In a three necked flask fitted with a magnetic stirrer and a dropping funnel, under nitrogen, S-[cyano(phenyl)methyl]ethanethioate a26 (11.934 g, 62.4 mmol) is dissolved in 56 mL of dry THF (distilled over sodium). The mixture is cooled to 0° C. and BH₃-THF complex (124.6 mL, 124.8 mmol, 1 M) is added dropwise. After stirring overnight at room temperature, the mixture is cooled again to 0° C. and methanol (125 mL) is slowly added. The mixture is stirred for 10 minutes and concentrated to give the crude 2-amino-1-phenylethanethiol a28 which is directly used in the next reaction.

1-aminohexane-2-thiol a29 may be synthesized according to the same method.

### 10.4 Synthesis of 5-phenyl-1,3-thiazolidin-2-one a30

In a flask fitted with a magnetic stirrer, 2-amino-1-phenylethanethiol a28 dissolved in 125 mL of toluene/distilled water (50/50 v/v) is cooled down to 0° C. and K₂CO₃ (25.867 g, 187.2 mmol) is added. Phosgene (70.51 mL, 124.8 mmol, as a solution 20% in toluene) is added dropwise. After one hour at 0° C., the mixture is extracted with Et₂O (3×60 mL), dried over anhydrous MgSO₄, concentrated under reduced pressure and purified by chromatography on silicagel (AcOEt/hexane 25/75 v/v) to afford 2.16 g of 5-phenyl-1,3-thiazolidin-2-one a30. Yield: 19% (from a26). LC-MS (MH⁺): 180.

5-butyl-1,3-thiazolidin-2-one a31 (LC-MS basic (MH⁺): 160) and 5-propyl-1,3-thiazolidin-2-one a32 (GC-MS (M⁺·): 145) may be synthesized according to the same method.

### 10.5 Synthesis of 2-(2-oxo-5-phenyl-1,3-thiazolidin-3-yl)butanamide 15

In a three necked flask fitted with a magnetic stirrer and a dropping funnel, under nitrogen atmosphere, 5-phenyl-1,3-thiazolidin-2-one a30 (0.984 g, 7.5 mmol) is dissolved in DMF (6 mL). NaH (0.343 g, 8.6 mmol, 60% dispersion in oil) is added by portions at 5° C. After stirring for 25 minutes at room temperature, 2-bromobutanamide (1.557 g, 9 mmol) is added by portions and the mixture is stirred overnight. Ice and water are added and the mixture is extracted with AcOEt (3×20 mL). The combined organic extracts are dried over anhydrous MgSO₄ and concentrated under reduced pressure to give the crude reaction mixture which is purified by chromatography on silicagel (CH₂Cl₂/C₂H₅OH 97.5/2.5 v/v). After recry-stallization from AcOEt/Hexane (70/30 v/v), 2-(2-oxo-5-phenyl-1,3-thiazolidin-3-yl)butanamide 15 is obtained as a white solid (0.435 g). Yield: 22%. LC-MS (MH⁺): 265. Compounds 11, 12, 13, 14 and 16 may be synthesized according to the same method.

### Example 11

### Synthesis of (2S)-2-[2-oxo-5-(2,2,2-trifluoroethyl)-1,3-thiazolidin-3-yl]butanamide 17

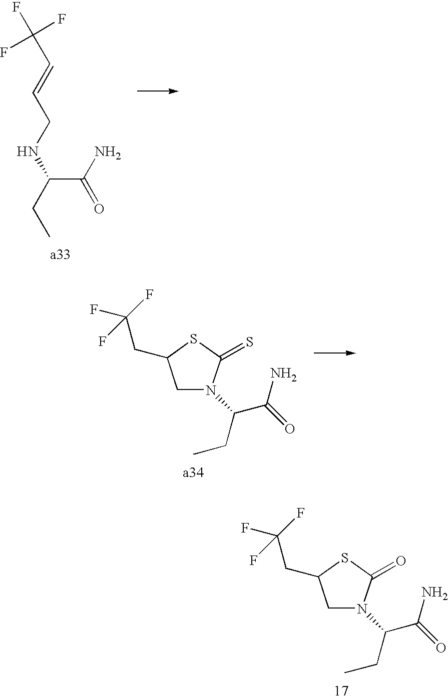

### 11.1 Synthesis of (2S)-2-[2-thioxo-5-(2,2,2-trifluoroethyl)-1,3-thiazolidin-3-yl]butanamide a34

In a flask fitted with a magnetic stirrer, (2S)-2-[(4,4,4-trifluorobut-2-en-1-yl)amino]butan-amide a33 (synthesized as described in patent application WO 2005/121082; 5.0 g, 23.8 mmol) is dissolved in DMF (50 mL). Cs₂CO₃ (8.52 g, 26.2 mmol), nBu₄NI (0.88 g, 2.38 mmol) and CS₂ (2.2 mL, 35.7 mmol) are added and the reaction mixture is stirred at room temperature for 1 hour. The salts are filtered and the filtrate is concentrated under reduced pressure. The crude reaction mixture is purified by chromatography on silicagel (AcOEt/hexane 50/50 v/v). The product is dissolved in CH₂Cl₂, the organic phase is washed with water (2×50 mL), the organic extract is dried over MgSO₄ and the solvent is concentrated in vacuo. The crude compound is purified successively by chromatography on silicagel (CH₂Cl₂/C₂H₅OH/NH₄OH 97.08/2/0.2 v/v/v), then by preparative HPLC on reverse phase (LC-prep-basic; column 30*50 mm xterra OBD 5 μm; gradient: CH₃CN/H₂O/hydrogenocarbonate) to afford 0.166 g of (2S)-2-[2-thioxo-5-(2,2,2-trifluoroethyl)-1,3-thiazolidin-3-yl]butanamide a34. Yield: 2%. LC-MS (MH⁺): 287.

### 11.2 Synthesis of (2S)-2-[2-oxo-5-(2,2,2-trifluoroethyl)-1,3-thiazolidin-3-yl]butanamide 17

In a flask fitted with a magnetic stirrer, (2S)-2-[2-thioxo-5-(2,2,2-trifluoroethyl)-1,3-thiazolidin-3-yl]butanamide a34 (2.5 g, 8.7 mmol), benzoic acid (1.06 g, 8.7 mmol) and benzyltriethylammonium chloride (0.198 g, 0.87 mmol) are dissolved in CH₂Cl₂ (250 mL). A solution of KMnO₄ (4.12 g, 26.1 mmol) in water (100 mL) is added. After vigorous stirring at room temperature for 16 hours, Na₂S₂O₅ is added to the mixture until the color has disappeared. The mixture is filtered and the aqueous phase is extracted with CH₂Cl₂. The organic extracts are dried over MgSO₄ and concentrated under reduced pressure. The crude reaction mixture is purified twice by chromatography on silicagel (eluent 1: hexane/AcOEt 70/30 v/v); eluent 2: CH₂Cl₂/C₂H₅OH/NH₄OH 91.2/8/0.8 v/v/v) and recrystallized from diisopropyl ether to afford (2S)-2-[2-oxo-5-(2,2,2-trifluoroethyl)-1,3-thiazolidin-3-yl]butanamide 17 (0.267 g). Yield: 11%. LC-MS (MH⁺): 271.

### Example 12

### Synthesis of 1-{[6-chloro-2-(trifluoromethyl)imidazo[1,2-b]pyridazin-3-yl]methyl}piperidin-2-one 18

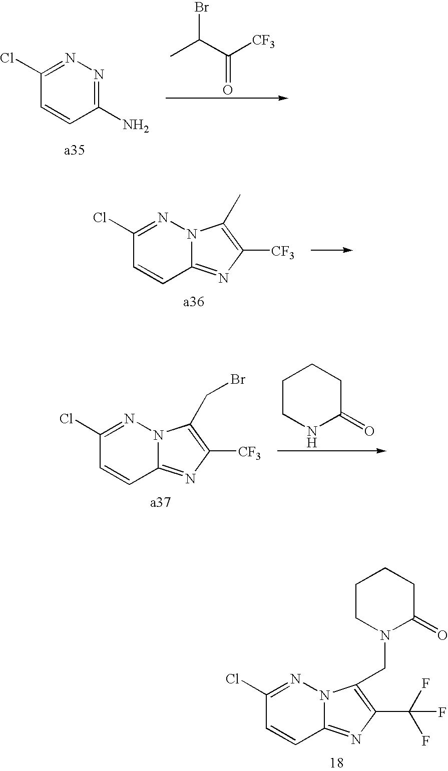

### 12.1 Synthesis of 6-chloro-3-methyl-2-(trifluoromethyl)imidazo[1,2-b]pyridazine a36

A solution of 6-chloro-3-aminopyridazine a35 (3.16 g, 24.2 mmol) and 3-bromo-1,1,1-trifluorobutan-2-one (5 g, 24.4 mmol) in 1,2-dimethoxyethane (100 mL) is refluxed during 17 hours. After cooling to room temperature and filtration, the solvent is removed under reduced pressure and the crude reaction mixture is purified by preparative chromatography on silicagel (CH₂Cl₂/MeOH/NH₄OH 98/2/0.2 v/v/v) to afford to 6-chloro-3-methyl-2-(trifluoromethyl)imidazo[1,2-b]pyridazine a36. Yield: 60%. LC-MS (MH⁺): 236/238.

### 12.2. Synthesis of 3-(bromomethyl)-6-chloro-2-(trifluoromethyl)imidazo[1,2-b]pyridazine a37

A mixture of 6-chloro-3-methyl-2-(trifluoromethyl)imidazo[1,2-b]pyridazine a36 (3.45 g, 1 eq, 14.64 mmol), N-bromosuccinimide (NBS; 2.87 g, 16.11 mmol) and azo-bis-iso-butyronitrile (AIBN; 240 mg, 1.5 mmol) in acetonitrile (50 mL) is refluxed for 2 hours. After cooling to room temperature, the solvent is removed under reduced pressure and the crude product is purified by preparative chromatography on silicagel (CH₂Cl₂) to afford 3-(bromomethyl)-6-chloro-2-(trifluoromethyl)imidazo[1,2-b]pyridazine a37. Yield: 35%. LC-MS (MH⁺): 315/317.

### 12.3. Synthesis of 1-{[6-chloro-2-(trifluoromethyl)imidazo[1,2-b]pyridazin-3-yl]methyl}-piperidin-2-one 18

In a three-necked flask fitted with a magnetic stirrer, NaH (60% in dispersion in mineral oil, 0.137 g, 3.4 mmol) is added to THF (10 mL). A solution of piperidin-2-one (250 mg, 0.25 mmol) in THF (5 mL) is added dropwise. A white solid appears and the mixture is stirred for 10 min at room temperature. A solution of 3-(bromomethyl)-6-chloro-2-(trifluoromethyl)imidazo[1,2-b]pyridazine a37 (0.720 g, 0.23 mmol) in THF (5 mL) is then added. After stirring at room temperature for 24 h, H₂O (10 mL) is added and the reaction mixture is extracted with ethyl acetate and with dichloromethane (twice). The combined organic phases are dried over MgSO₄, filtered and concentrated under reduced pressure.

The crude reaction mixture is purified by preparative chromatography on silicagel (CH₂Cl₂/MeOH/NH₄OH 98/1.8/0.2 v/v/v) and recrystallized from ethyl acetate to afford 1-{[6-chloro-2-(trifluoromethyl)imidazo[1,2-b]pyridazin-3-yl]methyl}piperidin-2-one 18 (0.248 g). Yield: 30%. LC-MS (MH⁺): 333/335.

Compound 29 may be synthesized according to the same method.

### Example 13

### Synthesis of 1-(1H-imidazol-1-ylmethyl)-5-phenylpiperidin-2-one 22

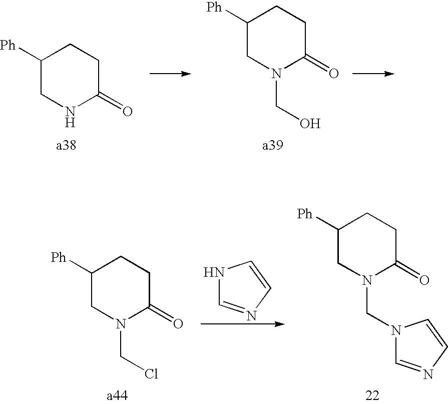

### 13.1 Synthesis of 1-(hydroxymethyl)-5-phenylpiperidin-2-one a39

KOH (9 mg, 0.16 mmol) is added to a suspension of 5-phenylpiperidin-2-one a38 (from Hill et al.; J. Am. Chem. Soc. (1959), 81, 737; 1.75 g, 10 mmol) in absolute dioxane (2 mL) in the absence of air moisture, and the reaction mixture is heated to 100-110° C. Paraformaldehyde (0.325 g, 10.8 mmol) is added in portions over 10 min. The formed homogeneous solution is stirred at this temperature for 30 min and cooled down to room temperature. The solvents are removed under reduced pressure, and the residue is reevaporated with chloroform and then with diethylether to give 2.1 g of 1-(hydroxymethyl)-5-phenylpiperidin-2-one a39 as a semisolid mass which is used in the next step without additional purification. Yield: 100%.

¹H NMR δ_{H} (DMSO, ppm): 1.97 (m, 3H), 2.37 (m, 2H), 3.07 (m, 1H), 4.69 (m, 1H), 4.77 (m, 1H), 5.77 (t, J=6.8 Hz, 1H), 7.25 (m, 1H), 7.32 (m, 5H).

The following compounds may be prepared according to the same method:

| | | |
|---|---|---|
| a40 | 6-fluoro-3- | GC-MS (M⁺·): 183. |
| | (hydroxymethyl)-1,3- | |
| | benzoxazol-2(3H)-one | |
| a41 | 1-(hydroxymethyl)-5- | ¹H NMR δ_{H} (CDCl₃, ppm): 0.92 (m, 3 H), |
| | propylpiperidin-2- | 1.37 (m, 5 H), 1.85 (m, 2 H), |
| | onedroxymethyl)-5- | 2.32-2.39 (m, 1 H), 2.41-2.51 (m, |
| | propylpiperidin-2-one | 1 H), 3.08 (m, 1 H), 3.42-3.48 (m, |
| | | 1 H), 4.74-4.96 (m, 2 H) |
| a42 | 1-(hydroxymethyl)-4- | ¹H NMR δ_{H} (DMSO, ppm): 1.90 (m, |
| | phenylpiperidin-2-one | 1 H), 2.03 (m, 1 H), 2.38 (m, 1 H), |
| | | 2.47 (m, 1 H), 3.05 (m, 1 H), |
| | | 3.42 (m, 2 H), 4.74 (s, |
| | | 2 H), 5.79 (s, 1 H), 7.29 (m, 5 H) |
| a43 | 1-(hydroxymethyl)-4- | ¹H NMR δ_{H} (DMSO, ppm): 0.87 (m, |
| | propylpiperidin-2-one | 3 H), 1.19-1.40 (m, 5 H), 1.73 (m, 1 H), |
| | | 1.86 (m, 1 H), 2.30 (m, 1 H), 3.30 (m, |
| | | 3 H), 4.67 (m, 2 H), 5.69 (m, 1 H) |

### 13.2 Synthesis of 1-(chloromethyl)-5-phenylpiperidin-2-one a44

SOCl₂ (11.8 g, 100 mmol) is added in one portion to a suspension of 1-(hydroxymethyl)-5-phenylpiperidin-2-one a39 (2.1 g, 10 mmol) in toluene (5 mL) at 0-5° C. in the absence of air. The homogeneous solution is stirred overnight allowing the mixture to gradually heat to room temperature. The excess of SOCl₂ and toluene is removed, and the residue is reevaporated with dichloromethane to give 1-(chloromethyl)-5-phenylpiperidin-2-one a44 as a light-yellow oil that recrystallized on keeping is used in the next step without additional purification.

¹H NMR δ_{H} (DMSO, ppm): 2.09 (m, 2H), 2.53 (m, 1H), 2.65 (m, 1H), 3.13 (m, 1H), 3.59 (d, J=8.3 Hz, 2H), 5.16 (d, J=9.3 Hz, 1H), 5.49 (d, J=9.3 Hz, 1H), 7.27 (m, 3H), 7.35 (m, 2H).

The following compounds may be prepared according to the same method:

| | | |
|---|---|---|
| a45 | 1-(chloromethyl)-5- | ¹H NMR δ_{H} (CDCl₃, ppm): 0.94 (m, 3 H), |
| | propylpiperidin- | 1.31-1.40 (m, 4 H), 1.47 (m, 1 H), |
| | 2-one | 1.81-1.96 (m, 2 H), 2.36 (m, 1 H), 2.52 (m, |
| | | 1 H), 3.14 (dd, J = 11.5, 10.3 Hz, 1 H), |
| | | 3.43 (ddd, J = 11.7, 4.9, |
| | | 2.0 Hz, 1 H), 5.14 (d, |
| | | J = 9.3 Hz, 1 H), 5.44 (d, J = 9.3 Hz, 1 H) |
| a46 | 1-(chloromethyl)- | ¹H NMR δ_{H} (CDCl₃, ppm): 0.94 (m, 3 H), |
| | 5-propylpiperidin- | 1.31-1.40 (m, 4 H), 1.47 (m, 1 H), |
| | 2-one | 1.81-1.96 (m, 2 H), 2.36 (m, 1 H), 2.52 (m, |
| | | 1 H), 3.14 (dd, J = 11.5, 10.3 Hz, 1 H), |
| | | 3.43 (ddd, J = 11.7, |
| | | 4.9, 2.0 Hz, 1 H), 5.14 (d, |
| | | J = 9.3 Hz, 1 H), 5.44 (d, J = 9.3 Hz, 1 H) |
| a47 | 1-(chloromethyl)- | ¹H NMR δ_{H} (CDCl₃, ppm): 2.06 (m, 1 H), |
| | 4-phenylpiperidin- | 2.21 (m, 1 H), 2.56 (m, 1 H), 2.80 (m, |
| | 2-one | 1 H), 3.14 (m, 1 H), 3.52 (m, 1 H), 3.61 (m, |
| | | 1 H), 5.24 (d, J = 9.3 Hz, 1 H), 7.20 (m, 2 |
| | | H), 7.26 (m, 1 H), 7.35 (m, 2 H), 5.48 (d, |
| | | J = 9.3 Hz, 1 H) |

### 13.3 Synthesis of 1-(1H-imidazol-1-ylmethyl)-5-phenylpiperidin-2-one 22

Imidazole (0.272 g, 4.0 mmol) is added at 0-5° C. under argon to a suspension of LiH (0.160 g, 20 mmol) in absolute DMF (22 mL). The mixture is stirred at this temperature for 45 min, and a solution of 1-(chloromethyl)-5-phenylpiperidin-2-one a44 (1.07 g, 4.8 mmol) in absolute DMF (6 mL) is added. The reaction mixture is stirred at 0-5° C. for 1 h and at room temperature for 1 h. The mixture is quenched with a saturated NH₄Cl solution (30 mL), diluted with water (30 mL), and subjected to extraction with dichloromethane. The organic extract is washed with water, dried over anhydrous Na₂SO₄, and evaporated. The oily residue (1.06 g) is purified by chromatography on silicagel (gradient AcOEt/MeOH from 20/1 to 10/1 v/v) to afford 1-(1H-imidazol-1-ylmethyl)-5-phenylpiperidin-2-one 22 as a viscous oil (363 mg). Yield: 35%. LC-MS (MH⁺): 256.

Compounds 20 and 25 may be prepared according to the same method.

Compounds 63 and 64 may be obtained using NaH instead of LiH and at room temperature.

### Example 14

### Synthesis of 1-(imidazo[1,2-a]pyridin-3-ylmethyl)-5-propylpiperidin-2-one 21

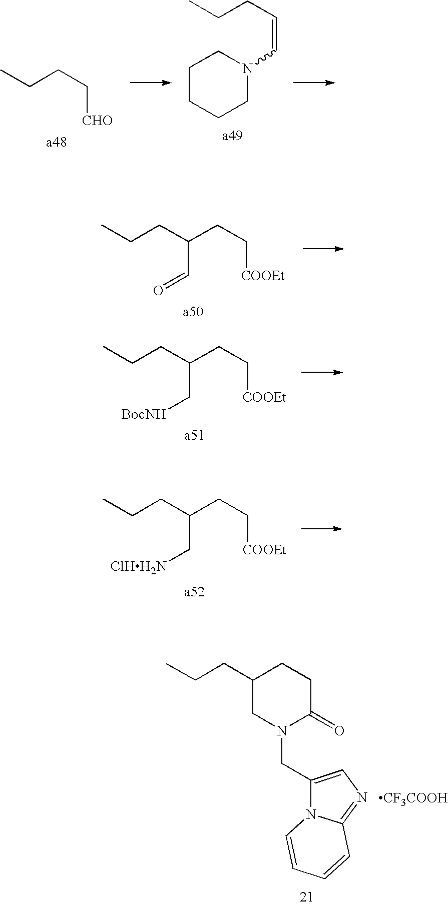

### 14.1 Synthesis of 1-(pent-1-en-1-yl)piperidine a49

A solution of pentanal a48 (4.03 g, 47 mmol), piperidine (3.99 g, 47 mmol) and 20 mg of pMePhSO₃H in benzene (35 mL) is refluxed with a Dean-Stark trap until water ceases to separate for approximately 2.5 h. The solvents are removed under reduced pressure to give 7.54 g of crude 1-(pent-1-en-1-yl)piperidine a49, which is used in the next step without additional purification. Yield: 100%.

¹H NMR δ_{H} (DMSO, ppm): 0.84 (t, J=7.3 Hz, 3H), 1.28 (m, 2H), 1.46 (m, 6H), 1.87 (m, 2H), 2.68 (m, 4H), 4.24 (m, 1H), 5.78 (d, J=13.7 Hz, 1H).

### 14.2 Synthesis of ethyl 4-formylheptanoate a50

A solution of ethyl acrylate (5.88 g, 58.8 mmol) in absolute acetonitrile (15 mL) is added dropwise under stirring in argon at 0-5° C. to a solution of crude 1-(pent-1-en-1-yl)piperidine a49 (7.54 g, 47 mmol) in absolute acetonitrile (35 mL) for 25 min. In 1 h, the reaction mixture is heated to 75-80° C., stirred for 25 h, and a solution of acetic acid (3 mL) in water (20 mL) is added. The reaction mixture is kept at 90° C. for 8 h, cooled to room temperature, saturated with NaCl and extracted with diethylether. The combined organic extracts are sequentially washed with 5% HCl, 5% NaHCO₃, and brine, dried over anhydrous Na₂SO₄ and evaporated. The residue (6.26 g) is purified by chromatography on silicagel (gradient hexane/AcOEt from 50/1 to 10/1 v/v) to give ethyl 4-formylheptanoate a50 (4.0 g). Yield: 46%.

¹H NMR δ_{H} (DMSO, ppm): 0.92 (t, J=7.3 Hz, 3H), 1.24 (t, J=7.1 Hz, 3H), 1.29-1.48 (m, 3H), 1.65 (m, 1H), 1.77 (m, 1H), 1.95 (m, 1H), 2.22-2.40 (m, 3H), 4.12 (q, J=7.1 Hz, 2H), 9.58 (d, J=2.4 Hz, 1H).

### 14.3 Synthesis of ethyl 4-{[(tert-butoxycarbonyl)amino]methyl}heptanoate a51

A solution of ethyl 4-formylheptanoate a50 (3.68 g, 19.8 mmol), tert-butyl carbamate (6.74 g, 57.5 mmol), triethylsilane (9.2 mL, 58 mmol), and TFA (3.05 mL, 38.3 mmol) in absolute acetonitrile (86 mL) is stirred at room temperature under argon for 16 h. The reaction mixture is diluted with diethylether (180 mL), washed with a 10% NaHCO₃ solution, and the organic layer is separated. The aqueous layer is additionally extracted with diethylether (50 mL), and the combined organic extracts are washed with brine (100 mL), dried over anhydrous Na₂SO₄, and evaporated. The residue (16.8 g) is purified by chromatography on silicagel (hexane/AcOEt 10/1 v/v) to give ethyl 4-{[(tert-butoxycarbonyl)amino]methyl}-heptanoate a51 (3.57 g). Yield: 62%.

¹H NMR δ_{H} (CDCl₃, ppm): 0.88 (t, J=7.1 Hz, 3H), 1.23 (m, 5H), 1.31 (m, 2H), 1.43 (s, 9H), 1.47 (m, 1H), 1.60 (m, 2H), 2.32 (t, J=7.6 Hz, 2H), 2.96-3.16 (m, 2H), 4.11 (q, J=7.1 Hz, 2H), 4.57 (m, 1H).

### 14.4 Synthesis of ethyl 4-(aminomethyl)heptanoate hydrochloride a52

4 N HCl in dioxane (20 mL, 80 mmol) is added to a solution of ethyl 4-{[(tert-butoxycarbonyl)amino]methyl}heptanoate a51 (3.57 g, 12.4 mmol) in absolute dioxane (20 mL), and the reaction mixture is stirred at room temperature overnight. The solvents are evaporated under reduced pressure, and the residue (3.2 g) is washed twice with hexane and reevaporated with diethylether to afford 2.5 g of ethyl 4-(aminomethyl)heptanoate hydrochloride a52 as a colorless oil. Yield: 90%.

¹H NMR δ_{H} (CDCl₃, ppm): 0.92 (t, J=6.6 Hz, 3H), 1.25 (t, J=7.1 Hz, 3H), 1.35 (m, 4H), 1.78 (m, 3H), 2.37 (t, J=6.8 Hz, 2H), 2.95 (m, 2H), 4.12 (q, J=7.1 Hz, 2H), 8.36 (m, 3H).

### 14.5 Synthesis of 1-(imidazo[1,2-a]pyridin-3-ylmethyl)-5-propylpiperidin-2-one trifluoroacetate 21

Triethylamine (0.27 mL, 1.95 mmol) is added to a solution of ethyl 4-(aminomethyl)-heptanoate hydrochloride a52 (0.435 g, 1.95 mmol) in absolute Et₂O (7 mL). The formed precipitate is separated by filtration, and the filtrate is evaporated under reduced pressure. The residue is dissolved in dichloromethane (3.6 mL), and imidazo[1,2-a]pyridine-3-carbaldehyde (0.284 g, 1.95 mmol) and Ti(O-iPr)₄ (0.94 mL, 3.14 mmol) are added to the obtained solution. The mixture is stirred at room temperature for 3 h, and the solvents are removed under reduced pressure. The residue is dissolved in absolute methanol (0.5 mL), and NaBH₄ (0.116 g, 3.06 mmol) is added under stirring. After 15 min, the reaction mixture is quenched by the addition of a 0.1 N NaOH solution (5 mL). The mixture is stirred for 10 min, diluted with dichloromethane (20 mL), and passed through a Celite path. Celite is washed with dichloromethane, and the filtrate is evaporated. The residue (0.58 g) is subjected to rough purification by flash chromatography on silicagel (chloroform/methanol 50/1 v/v) to give a crude product. According to the ¹H NMR and LC-MS data, this product consists of the mixture of compound 21, its non-cyclized form (corresponding aminoester), and 5-propylvalerolactame. This mixture is dissolved in toluene (3 mL) and refluxed for 4.5 h. Toluene is removed under reduced pressure, and the residue is purified by reverse phase HPLC (C18 column; gradient acetonitrile/water/TFA from 20/80/0.1 to 85/15/0.1 v/v/v) to give 1-(imidazo[1,2-a]pyridin-3-ylmethyl)-5-propylpiperidin-2-one trifluoroacetate 21 (0.149 g). Yield: 20%. LC-MS (MH⁺): 272.

Compound 19 may be obtained according to the same method.

### Example 15

### Synthesis of 1-(imidazo[1,2-a]pyridin-3-ylmethyl)-5-phenylpiperidin-2-one 23

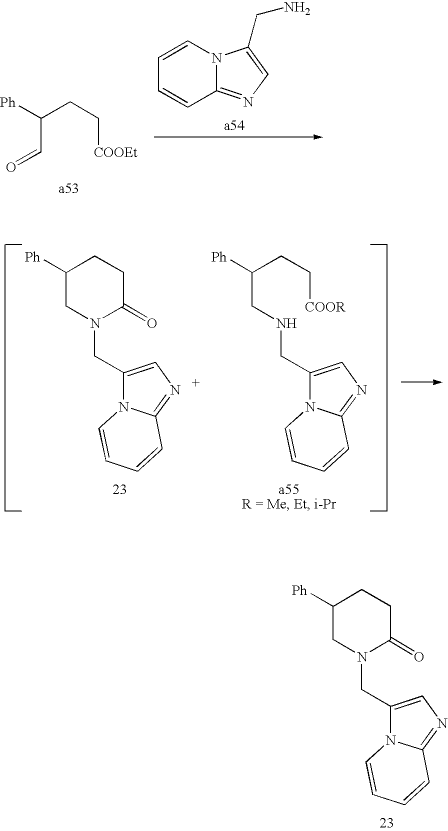

1-imidazo[1,2-a]pyridin-3-ylmethanamine a54 (197 mg, 1.33 mmol) and Ti(O-i-Pr)₄ (0.713 mL, 2.37 mmol) are added to a solution of ethyl 5-oxo-4-phenylpentanoate a53 (see Ledon H. et al., Bull. Soc. Chim. Fr. (1973), 2071-2076; 294 mg, 1.33 mmol) in dichloromethane (2.5 mL) under stirring in argon. The reaction mixture is stirred at room temperature for 3.5 h, and an additional portion of Ti(O-i-Pr)₄ (0.08 mL, 0.37 mmol) is added. After 40 min., the mixture is evaporated under reduced pressure. The residue is dissolved in absolute methanol (6 mL), and NaBH₄ (81 mg, 2.1 mmol) is added in portions under stirring. After 15 min., a 0.1 N NaOH solution (5 mL) is added, and the mixture is additionally stirred for 15 min. and many times subjected to extraction with dichloromethane. The combined organic extracts are dried over anhydrous Na₂SO₄ and evaporated to afford an oily residue (466 mg). According to the LC-MS data, this residue mainly consisted of the mixture of methyl, ethyl, and isopropyl esters a55. The residue is dissolved in toluene (10 mL) and refluxed for 9 h. The reaction mixture is cooled to room temperature and evaporated under reduced pressure. The residue (459 mg) is purified by chromatography on silicagel (chloroform/methanol 100/5 v/v) to give crude compound 23 (purity of 72%; 285 mg), which is purified several times by chromatography on silicagel (hexane/AcOEt 1/1 v/v) to afford pure 1-(imidazo[1,2-a]pyridin-3-ylmethyl)-5-phenylpiperidin-2-one 23 (110 mg).

Yield: 27%. LC-MS (MH⁺): 306.

### Example 16

### Synthesis of 1-(imidazo[1,2-a]pyridin-3-ylmethyl)-4-phenylpiperidin-2-one hydrochloride 24

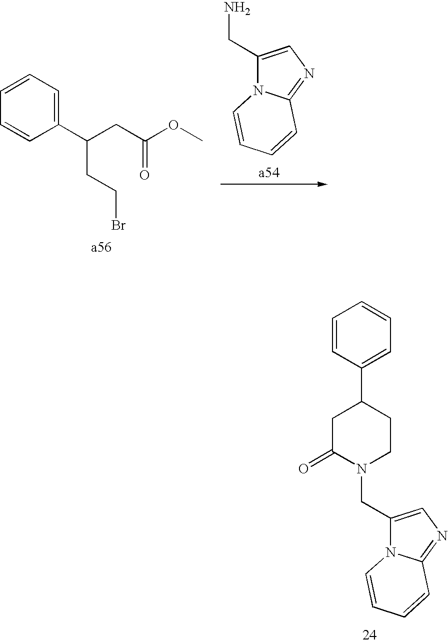

A mixture of ethyl 5-bromo-3-phenylpentanoate a56 (synthesized as described by Burger and Hofstetter in J. Org. Chem. (1959), 24, 1290); 0.425 g, 1.49 mmol), 1-imidazo[1,2-a]pyridin-3-ylmethanamine a54 (0.329 g, 2.24 mmol), and anhydrous Cs₂CO₃ (0.329 g, 2.24 mmol) in absolute MeCN (6 mL) is refluxed under stirring in the absence of air moisture for 5 h, cooled to room temperature, filtered, and evaporated. The residue (0.57 g) is purified twice by chromatography on silicagel (eluent 1: CHCl₃/MeOH 20/1 v/v; eluent 2: AcOEt/acetone 1/1 v/v). The obtained fraction is dissolved in THF (0.5 mL) and 4 M HCl in dioxane (0.15 mL) is added to the obtained solution to give, after filtration, 1-(imidazo[1,2-a]pyridin-3-ylmethyl)-4-phenylpiperidin-2-one hydrochloride 24 (0.082 g) as a solid.

Yield: 16%. LC-MS (MH⁺): 306.

1-(imidazo[1,2-a]pyridin-3-ylmethyl)-4-propylpiperidin-2-one hydrochloride 26 and 1-(1H-imidazol-5-ylmethyl)-4-propylpiperidin-2-one 27 are synthesized according to the same method, starting from ethyl 3-(2-bromoethyl)hexanoate a57 (synthesized in the racemic form using a similar method to the one described by Jones J. B. and Lok K. P. in Can. J. Chem. (1979), 57, 1025-1032).

### Example 17

### Synthesis of 1-(1H-imidazol-5-ylmethyl)-5-propylazepan-2-one 30, 1-(1H-imidazol-5-ylmethyl)-6-propylazepan-2-one 33 and 1-(1H-imidazol-4-ylmethyl)-4-propylazepan-2-one 34

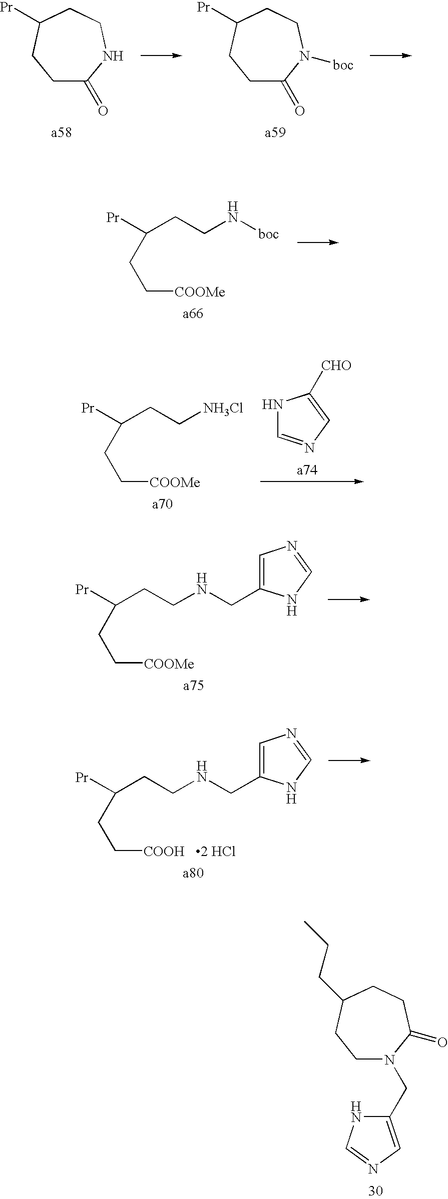

### 17.1 Synthesis of tert-butyl 2-oxopropylazepane-1-carboxylates a59, a64 and a65

### 17.1.1 Synthesis of tert-butyl 2-oxo-5-propylazepane-1-carboxylate a59

A solution of 5-propylazepan-2-one a58 (2.96 g, 19 mmol) Boc₂O (6.24 g, 28.6 mmol), and DMAP (2.55 g, 20.9 mmol) in absolute THF (60 mL) is stirred in a flow of argon at room temperature for 16 h. The reaction mixture is evaporated in vacuo. The residue (9.4 g) is purified by flash-chromatography on silicagel (chloroform). Tert-butyl 2-oxo-5-propyl-azepane-1-carboxylate a59 (4.50 g) is obtained as a pale-yellow oil. Yield: 92.5%. LC-MS (MH⁺): 156.

Tert-butyl 2-oxo-5-phenylazepane-1-carboxylate a60 may be synthesized according to the same method.

¹H NMR δ_{H} (DMSO, ppm): 1.45 (s, 9H), 1.52-1.77 (m, 2H), 1.90 (m, 1H), 2.01 (m, 1H), 2.47 (m, 1H), 2.84 (m, 1H), 2.97 (m, 1H), 3.58 (m, 1H), 4.13 (m, 1H), 7.24 (m, 5H).

### 17.1.2 Synthesis of tert-butyl 2-oxo-4-propyl-1-azepanecarboxylate a64 and tert-butyl 2-oxo-6-propyl-1-azepanecarboxylate a65

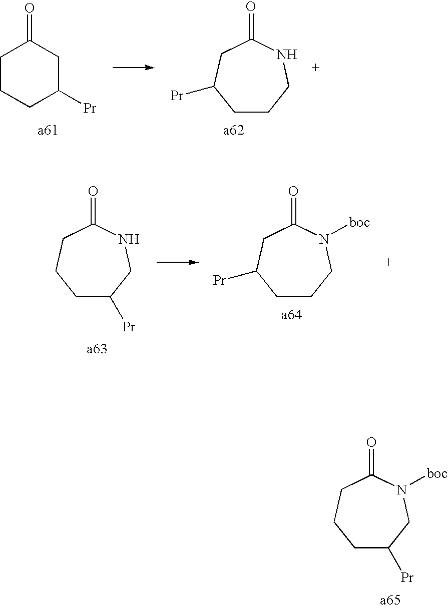

### (i) Synthesis of 4-propylazepan-2-one a62 and 6-propylazepan-2-one a63

3-propylcyclohexanone a61 (1.0 g, 7.13 mmol; from H. O. House, W. F. Fischer, J. Org. Chem. (1969), 34, 3615-3618) is dissolved in chloroform (12.5 mL). Sodium azide (1.4 g, 21.4 mmol) is added under stirring. After cooling to 0-5° C. a solution of methanesulfonic acid (6.85 g, 71.3 mmol) in chloroform (4.5 mL) is added dropwise. The cooling bath is removed. The reaction mixture is stirred for 3 h at room temperature. A saturated solution of NaHCO₃ is added to attain pH 7. The organic layer is separated. The aqueous one is additionally extracted with chloroform. The combined extracts are dried over anhydrous MgSO₄ and evaporated in vacuo. The residue is purified by chromatography on silicagel (AcOEt) to afford a 0.70 g of a 1:1 mixture of regioisomers 4-propylazepan-2-one a62 and 6-propylazepan-2-one a63. Yield: 63%.

### (ii) Synthesis of tert-butyl 2-oxo-4-propylazepane-1-carboxylate a64 and tert-butyl 2-oxo-6-propylazepane-1-carboxylate a65

A solution of the mixture of regioisomers a62 and a63 (0.555 g, 3.57 mmol), DMAP (0.48 g, 3.93 mmol) and Boc₂O (0.86 g, 3.93 mmol) in absolute THF (9 mL) is stirred at room temperature for 48 h. The solvents are evaporated at room temperature. The residue is purified by chromatography on silicagel (EtOAc/hexane 1:8) to afford of tert-butyl 2-oxo-4-propylazepane-1-carboxylate a64 (0.36 g) and tert-butyl 2-oxo-6-propylazepane-1-carboxylate a65 (0.28 g).

Compound a64: Yield: 39.5%.

¹H NMR δ_{H} (CDCl₃, ppm): 0.91 (t, J=7.1 Hz, 3H), 1.18-1.44 (m, 5H), 1.52 (s, 9H), 1.54-1.73 (m, 2H), 1.83-1.93 (m, 2H), 2.62 (m, 2H), 3.26 (m, 1H), 3.94 (m, 1H).

Compound a65: Yield: 30.5%.

¹H NMR δ_{H} (CDCl₃, ppm): 0.89 (t, J=7.1 Hz, 3H), 1.24-1.40 (m, 5H), 1.52 (s, 9H), 1.54-1.63 (m, 1H), 1.69-1.79 (m, 1H), 1.83-1.95 (m, 2H), 2.54 (m, 2H), 3.46 (m, 1H), 4.65 (m, 1H).

### 17.2 Synthesis of methyl 4-{2-[(tert-butoxycarbonyl)amino]ethyl}heptanoate a66

Tert-butyl 2-oxo-5-propylazepane-1-carboxylate a59 (4.69 g, 18.4 mmol) is dissolved in absolute methanol (10 mL). A solution of sodium methoxide (1.098 g, 20.3 mmol) in absolute methanol (13 mL) is added under a flow of argon while stirring at 0-5° C. for 15 min. The cooling bath is removed. The reaction mixture was stirred at room temperature for 1 h, diluted with brine, and extracted with diethylether. The organic layer is dried over anhydrous Na₂SO₄ and evaporated to afford methyl 4-{2-[(tert-butoxycarbonyl)amino]-ethyl}heptanoate a66 (4.64 g) as a pale-yellow oil. Yield: 88%. LC-MS (MH⁺): 288.

The following compounds may be prepared according to the same method:

| | | |
|---|---|---|
| a67 | methyl 6-[(tert- | ¹H NMR δ_{H} (DMSO, ppm): 1.35 (s, 9 H), |
| | butoxycarbonyl)- | 1.59-1.79 (m, 3 H), 1.89 (m, 1 H), |
| | amino]-4- | 2.07 (m, 2 H), 2.55 (m, 1 H), 2.73 (m, 2 H), |
| | phenylhexanoate | 3.52 (s, 3 H), 6.71 (s (broad), 1 H), |
| | | 7.18 (m, 2 H), 7.30 (m, 3 H) |
| a68 | methyl 5-{[(tert- | ¹H NMR δ_{H} (DMSO, ppm): 0.88 (m, 3 |
| | butoxycarbonyl)- | H), 1.29 (m, 7 H), 1.44 (s, 9 H), 1.47 (m, |
| | amino]methyl}- | 1 H), 1.87 (m, 1 H), 2.24 (m, 2 H), |
| | octanoate | 3.09 (m, 2 H), 3.66 (s, 3 H). |
| | | LC-MS (MH⁺): 288. |
| a69 | methyl 6-[(tert- | ¹H NMR δ_{H} (DMSO, ppm): 0.89 (m, 3 |
| | butoxycarbonyl)- | H), 1.27 (m, 7 H), 1.44 (s, 9 H), 1.63 (m, |
| | amino]-3- | 2 H), 2.30 (m, 2 H), 3.06 (m, 2 H), |
| | propylhexanoate | 3.67 (s, 3 H), 4.52 (s (broad), 1 H). |
| | | LC-MS (MH⁺): 288. |

### 17.3 Synthesis of methyl 4-(2-aminoethyl)heptanoate hydrochloride a70

The reaction is carried out under anhydrous conditions. Methyl 4-{2-[(tert-butoxycarbonyl)-amino]ethyl}heptanoate a66 (4.64 g, 16.0 mmol) is dissolved in absolute dioxane (15 mL). A 4 N solution of HCl in dioxane (28 mL, 112 mmol) is added. The reaction mixture is stirred at room temperature overnight. The solvents are evaporated in vacuo. The residue is washed with hexane twice and subjected to coevaporation with diethylether. Methyl 4-(2-aminoethyl)heptanoate hydrochloride a70 (3.95 g) is obtained as a colorless oil. Yield: 98%. LC-MS (MH⁺): 188.

The following compounds may be prepared according to the same method:

| | | |
|---|---|---|
| a71 | methyl 6-amino- | ¹H NMR δ_{H} (DMSO, ppm): 1.74-1.96 (m, 4 |
| | 4-phenylhexanoate | H), 2.01-2.17 (m, 2 H), 2.44 (m, 1 H), |
| | | 2.64 (m, 2 H), 3.53 (s, 3 H), 7.22 (m, 3 H), |
| | | 7.31 (m, 2 H), 7.93 (s (broad), 3 H) |
| a72 | methyl 5- | ¹H NMR δ_{H} (DMSO, ppm): 0.85 (m, 3 H), |
| | (aminomethyl)- | 1.17-1.34 (m, 6 H), 1.53 (m, 2 H), 1.77 (m, |
| | octanoate | 1 H), 2.24 (m, 2 H), 2.72 (m, 2 H), |
| | hydrochloride | 3.59 (s, 3 H), 7.94 (s (broad), 3H). |
| | | LC-MS (MH⁺): 188. |
| a73 | methyl 6-amino- | ¹H NMR δ_{H} (DMSO, ppm): 0.87 (m, 3 H), |
| | 3-propylhexanoate | 1.17-1.34 (m, 6 H), 1.50 (m, 2 H), 1.63 (m, |
| | hydrochloride | 1 H), 2.30 (m, 2 H), 2.67 (d, J = 6.36 Hz, |
| | | 2 H), 3.59 (s, 3 H), 7.95 (s (broad), 3 H). |
| | | LC-MS (MH⁺): 188. |

### 17.4 Synthesis of methyl 4-{2-[(1H-imidazol-4-ylmethyl)amino]ethyl}heptanoate a75

Methyl 4-(2-aminoethyl)heptanoate hydrochloride a70 (0.447 g, 2.0 mmol) is suspended in absolute diethylether (2.5 mL). DIEA (diisopropylethylamine) (0.35 mL, 2.0 mmol) is added, and after 5 min the resulting mixture is evaporated in vacuo. The residue is dissolved in dichloromethane (2 mL). The obtained solution is added to a preliminarily prepared solution of 4-formylimidazole (0.192 g, 2.0 mmol) and Ti(O-i-Pr)₄ (0.96 mL, 3.2 mmol) in dichloromethane (5 mL). The reaction mixture is stirred at room temperature for 3 h and evaporated in vacuo. The residue is dissolved in absolute MeOH (5 mL). NaBH₄ (0.114 g, 3.0 mmol) is added under stirring. After 1 h the reaction mixture is diluted with dichloro-methane (50 mL). The solution is washed with a saturated solution of NaHCO₃.

The aqueous layer is extracted with dichloromethane. The combined extracts are dried over anhydrous Na₂SO₄ and evaporated to afford methyl 4-{2-[(1H-imidazol-4-ylmethyl)amino]-ethyl}heptanoate a75 (0.498 g) which is used in the next step without additional purification.

Yield: 93%. LC-MS: (MH⁺): 268.

The following compounds may be prepared according to the same method:

| | | |
|---|---|---|
| a76 | isopropyl 4-[2-({[2- | ¹H NMR δ_{H} (DMSO, ppm): 0.78 (m, 3 |
| | (trifluoromethyl)imidazo[1,2-a]pyridin-3- | H), 1.09 (m, 2 H), 1.13 (m, 7 H), |
| | yl]methyl}amino)ethyl]heptanoate | 1.30 (m, 3 H), 1.40 (m, 2 H), 2.15 (m, 3 H), |
| | | 2.46 (m, 2 H), 4.16 (s, 2 H), 4.84 (m, 1 |
| | | H), 7.07 (m, 1 H), 7.43 (m, 1 H), |
| | | 7.66 (m, 1 H), 8.58 (m, 1 H) |
| a77 | isopropyl 4-phenyl-6-({[2- | ¹H NMR δ_{H} (DMSO, ppm): 1.10 (d, J = 6.1, Hz, |
| | (trifluoromethyl)imidazo[1,2-a]pyridin-3- | 3 H), 1.12 (d, J = 6.3 Hz, 3 |
| | yl]methyl}amino)hexanoate | H), 1.56-1.85 (m, 4 H), 1.95 (m, 2 H), |
| | | 2.24 (m, 1 H), 2.34 (m, 1 H), 2.56 (m, |
| | | 1 H), 4.10 (m, 2 H), 4.81 (m, 1 H), |
| | | 7.0 (m, 2 H), 7.08-7.21 (m, 4 H), 7.45 (m, |
| | | 1 H), 7.66 (m, 1 H), 8.55 (m, 1 H) |
| a78 | methyl 5-{[(1H-imidazol-4- | LC-MS (MH⁺): 268 |
| | ylmethyl)amino]methyl}octanoate | |
| a79 | methyl 6-[(1H-imidazol-4-ylmethyl)amino]- | LC-MS (MH⁺): 268 |
| | 3-propylhexanoate | |

### 17.5 Synthesis of 4-{2-[(1H-imidazol-4-ylmethyl)amino]ethyl}heptanoic acid dihydrochloride a80

A solution of methyl 4-{2-[(1H-imidazol-4-ylmethyl)amino]ethyl}heptanoate a75 (0.440 g, 1.65 mmol) in 6 N HCl (3 mL) is stirred at 50-55° C. for 5.5 h. The reaction mixture is cooled to room temperature. The water is evaporated in vacuo. The residue is vacuum-dried to afford 0.538 g of crude 4-{2-[(1H-imidazol-4-ylmethyl)amino]ethyl}heptanoic acid dihydrochloride a80 (0.538 g). Yield: 100%. LC-MS: (MH⁺): 254.

The following compounds may be prepared according to the same method:

| | | |
|---|---|---|
| a81 | 4-[2-({[2-(trifluoromethyl)imidazo[1,2-a]pyridin-3- | LC-MS (MH⁺): 372 |
| | yl]methyl}amino)ethyl]heptanoic acid dihydrochloride | |
| a82 | 4-phenyl-6-({[2-(trifluoromethyl)imidazo[1,2-a]pyridin-3- | LC-MS (MH⁺): 406 |
| | yl]methyl}amino)hexanoic acid dihydrochloride | |
| a83 | 5-{[(1H-imidazol-4-ylmethyl)amino]methyl}octanoic acid | LC-MS (MH⁺): 254 |
| | dihydrochloride | |
| a84 | 6-[(1H-imidazol-4-ylmethyl)amino]-3-propylhexanoic acid | LC-MS (MH⁺): 254 |
| | dihydrochloride | |

### 17.6 Synthesis of 1-(1H-imidazol-5-ylmethyl)-5-propylazepan-2-one 30

The reaction is carried out under anhydrous conditions. Crude 4-{2-[(1H-imidazol-4-ylmethyl)amino]ethyl}heptanoic acid dihydrochloride a80 (0.538 g, 1.65 mmol) and DIEA (1.15 mm, 6.6 mmol) are dissolved in absolute DMF (100 mL), and TBTU (2-(1H-benzotriazol-1-yl)-1,1,3,3-tetramethyluronium tetrafluoroborate) (0.582 g, 1.82 mmol) is added. The reaction mixture is stirred at room temperature for 24 h and evaporated in vacuo. The residue is purified by chromatography on alumina (150 g; gradient CHCl₃/MeOH from 50/1 to 10/1 v/v) and by chromatography on silicagel (gradient EtOAc/MeOH from 10/1 to 4/1 v/v) affording pure 1-(1H-imidazol-5-ylmethyl)-5-propylazepan-2-one 30 (0.202 g). Yield: 52%. LC-MS (MH⁺): 236.

Compounds 31, 32, 33 and 34 may be prepared according to the same method.

### Example 18

### Synthesis of 2-(5-oxo-5,6-dihydro-4H-thieno[3,2-b]pyrrol-4-yl)acetamide 36

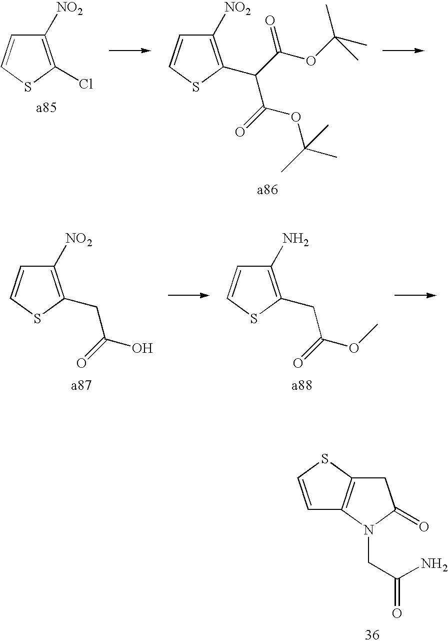

### 18.1 Synthesis of di-tert-butyl(3-nitro-2-thienyl)malonate a86

In a one necked flask fitted with a magnetic stirrer, under inert atmosphere, di(tert-butyl)malonate (10.53 g, 48.6 mmol) is dissolved in DMSO (200 mL) and NaH (1.94 g, 48.6 mmol, 60% dispersion in oil) is added at room temperature. The mixture is heated up to 100° C. for 1 hour to obtain a clear solution then cooled down to 20° C. and the 2-chloro-3-nitrothiophene a85 (528 g, 32.4 mmol) is added in one portion. The red solution is heated at 60° C. for 2 hours. The mixture is cooled down and water is slowly added. The mixture is diluted with CH₂Cl₂, HCl (0.1M) is added until a yellow solution is observed. The aqueous phase is extracted by CH₂Cl₂ (3×200 mL) and the combined organic extracts are dried over anhydrous MgSO₄ and concentrated under reduced pressure to give a brown oil which is purified by chromatography on silicagel (CH₂Cl₂/hexane 40/60 v/v) to afford di-tert-butyl(3-nitro-2-thienyl)malonate a86 (6.1 g), which is directly used in the next reaction.

Yield: 55%.

### 18.2 Synthesis of (3-nitro-2-thienyl)acetic acid a87

In a three necked flask fitted with a magnetic stirrer and a reflux condenser, under nitrogen atmosphere, di(tert-butyl)2-(3-nitro-2-thienyl)malonate a86 (6.1 g, 17.8 mmol) is dissolved in TFA (50 mL) and the solution is brought to reflux for 2 hours. The mixture is cooled down and the TFA is removed under reduced pressure to give (3-nitro-2-thienyl)acetic acid a87 as a yellow solid (3.3 g). Yield: 100%. SM (DIP, M⁺·): 187.

### 18.3 Synthesis of methyl(3-amino-2-thienyl)acetate a88

In a one necked flask fitted with a magnetic stirrer and a reflux condenser, (3-nitro-2-thienyl)acetic acid a87 (3.3 g, 17.65 mmol) is dissolved in 25 mL of HCl (37% w/w)/CH₃OH (1/1, v/v). At room temperature, Sn (6.24 g, 52.95 mmol) is added in one portion and the mixture is brought to reflux for 2 hours. After stirring at room temperature for 10 hours, the mixture is concentrated under reduced pressure to give a brown solid which is dissolved in acetonitrile. Et₃N (20 mL) is added and the precipitate is filtered off. After concentration of the filtrate, the residue is purified by chromatography on silicagel (gradient CH₂Cl₂/ACOEt 100/0 to 0/100 v/v) to give methyl(3-amino-2-thienyl)acetate a88 as a brown oil (2.7 g). Yield: 90%.

¹H NMR δ_{H} (CDCl₃, ppm): 3.55-3.80 (m, 5H); 6.56-6.61 (d, 1H); 6.98-7.20 (d, 1H).

### 18.4 Synthesis of 2-(5-oxo-5,6-dihydro-4H-thieno[3,2-b]pyrrol-4-yl)acetamide 36

In a sealed micro-wave tube, methyl(3-amino-2-thienyl)acetate a88 (0.5 g, 2.92 mmol) is dissolved THF (4 mL) and the mixture is submitted to 200 W at 100° C. for 50 minutes. The solvent is removed and the obtained crude product is first purified by chromatography on silicagel (AcOEt), the by reverse phase HPLC (column C18 OBD 30*50 mm; gradient H₂O/CH₃CN/TFA v/v/v) to afford 2-(5-oxo-5,6-dihydro-4H-thieno[3,2-b]pyrrol-4-yl)acetamide 36 (0.069 g) as brown solid. Yield: 12%. LC-MS (MH⁺): 197.

### Example 19

### Synthesis of 4-(1H-imidazol-4-ylmethyl)-4,6-dihydro-5H-thieno[3,2-b]pyrrol-5-one 35

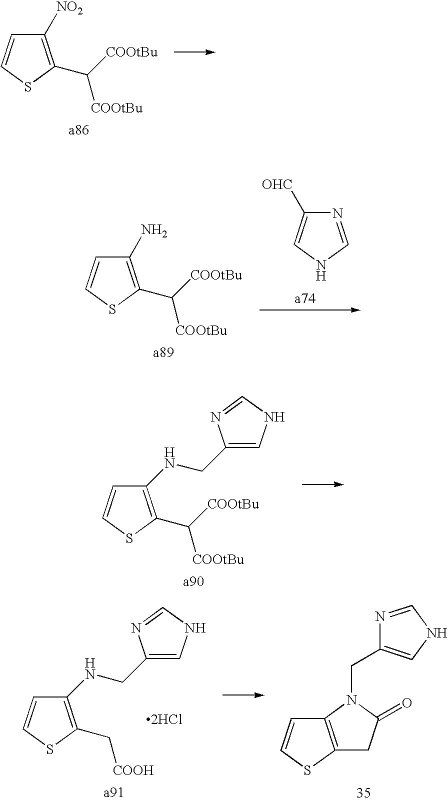

### 19.1 Synthesis of di-tert-butyl(3-amino-2-thienyl)malonate a89

A solution of di-tert-butyl(3-nitro-2-thienyl)malonate a86 (343 mg, 1.0 mmol) in ethyl acetate (5 mL) is hydrogenated in a Parr apparatus under a hydrogen pressure of 80 psi in the presence of 10% Pd/C (34 mg) at room temperature for 24 h. the reaction mixture is filtered through a Celite layer, and Celite is washed with ethyl acetate. The combined filtrates are evaporated to afford di-tert-butyl(3-amino-2-thienyl)malonate intermediate a89 as a yellow oil (300 mg) with a purity of 90%. Yield: 95%.

¹H NMR δ_{H} (DMSO, ppm): 1.42 (s, 18H), 4.81-4.86 (m, 3H), 6.49 (d, J=5.4 Hz, 1H), 7.15 (d, J=5.4 Hz, 1H).

### 19.2 Synthesis of di-tert-butyl{3-[(1H-imidazol-4-ylmethyl)amino]-2-thienyl}malonate a90

A mixture of di-tert-butyl(3-amino-2-thienyl)malonate a89 (1.0 g, 3.2 mmol) and 4-formylimidazole (0.306 g, 3.2 mmol) in trimethyl orthoformate (16 mL) is stirred at room temperature for 20 h. Trimethyl orthoformate is evaporated under reduced pressure, the residue is dissolved in methanol, and NaBH₄ (0.243 g, 6.4 mmol) is added in portions.

Then the reaction mixture is stirred at room temperature for 16 h and evaporated to dryness. The residue is distributed between water (10 mL) and chloroform (50 mL). The organic layer is separated, dried over anhydrous Na₂SO₄, and evaporated. The residue (1.49 g) is purified by chromatography on silicagel (chloroform/methanol 10/1 v/v) to give di-tert-butyl {3-[(1H-imidazol-4-ylmethyl)amino]-2-thienyl}malonate a90 (0.526 g). Yield: 42%.

¹H NMR δ_{H} (DMSO, ppm)1.41 (s, 18H), 4.14 (d, J=5.9 Hz, 2H), 4.91 (s, 1H), 6.74 (d, J=5.6 Hz, 1H), 6.83 (s, 1H), 7.23 (d, J=5.4 Hz, 1H), 7.53 (d, J=1.0 Hz, 1H), 11.82 (m, 1H).

### 19.3 Synthesis of {3-[(1H-imidazol-4-ylmethyl)amino]-2-thienyl}acetic acid dihydrochloride a91

A mixture of di-tert-butyl {3-[(1H-imidazol-4-ylmethyl)amino]-2-thienyl}malonate a90 (0.52 g, 1.32 mmol) and 6 N HCl (9 mL) is stirred at 70-75° C. for 45 min. The formed brown solution is cooled down to room temperature and evaporated to dryness under reduced pressure. The residue is several times reevaporated with benzene and vacuum-dried to give crude {3-[(1H-imidazol-4-ylmethyl)amino]-2-thienyl}acetic acid dihydrochloride a91 (0.42 g) which is used as such in the next step.

LC-MS (MH⁺): 236.

### 19.4 Synthesis of 4-(1H-imidazol-4-ylmethyl)-4,6-dihydro-5H-thieno[3,2-b]pyrrol-5-one 35

DIEA (1.4 mL, 7.92 mmol) is added to a suspension of {3-[(1H-imidazol-4-ylmethyl)amino]-2-thienyl}acetic acid dihydrochloride a91 (0.42 g, 1.32 mmol) in dichloroethane (14 mL) under stirring, and TBTU (0.466 g, 1.45 mmol) is added in 1 h. The reaction mixture is stirred overnight, and the solvents are evaporated. A 20% K₂CO₃ solution is added to the residue, and the mixture is subjected to extraction with chloroform. The combined extracts are dried over anhydrous Na₂SO₄ and evaporated. The residue (0.61 g) is purified by chromatography on silicagel (chloroform/methanol 10/1 v/v) to afford 0.125 g of 4-(1H-imidazol-4-ylmethyl)-4,6-dihydro-5H-thieno[3,2-b]pyrrol-5-one 35. Yield: 43%.

LC-MS (MH⁺): 220.

### Example 20

### Synthesis of 4-{[2-(trifluoromethyl)imidazo[1,2-a]pyridin-3-yl]methyl}-4,6-dihydro-5H-thieno[3,2-b]pyrrol-5-one 37

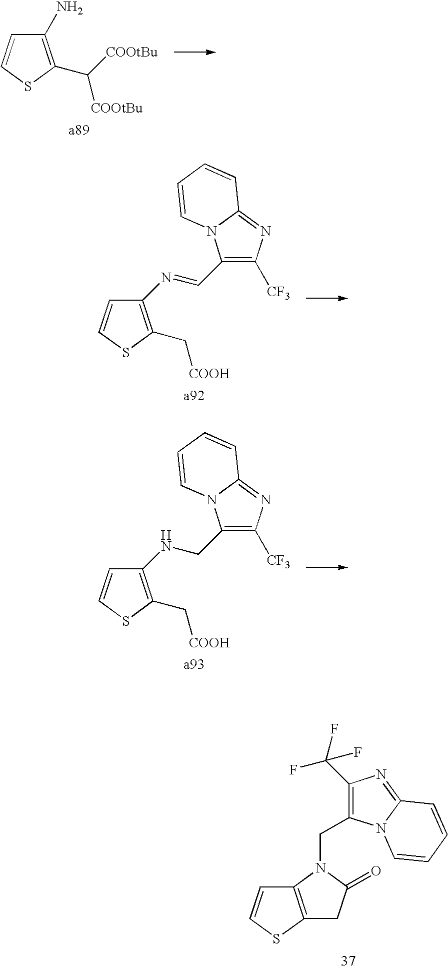

### 20.1 Synthesis of [3-({[2-(trifluoromethyl)imidazo[1,2-a]pyridin-3-yl]methylene}amino)-2-thienyl]acetic acid a92

A solution of 2-(trifluoromethyl)imidazo[1,2-a]pyridine-3-carbaldehyde (300 mg, 1.4 mmol) in dichloroethane (6 mL) is added under stirring to a solution of di-tert-butyl(3-amino-2-thienyl)malonate a89 (220 mg, 0.7 mmol) in a mixture of dichloroethane/TFA (40 mL, 1/1), and triethylsilane (0.455 mL, 2.8 mmol) is added in 10 min. The reaction mixture is stirred at room temperature for 2 h, and triethylsilane (0.215 mL, 1.2 mmol) is additionally added. The mixture is stirred at 35-40° C. for 3 h, and the solvents are removed under reduced pressure. The solid residue is washed with hexane and purified by chromatography on silicagel (chloroform/methanol 10/1) to give 160 mg of [3-({[2-(trifluoromethyl)imidazo[1,2-a]pyridin-3-yl]methylene}amino)-2-thienyl]acetic acid a92 with a purity of 85% (containing 15 wt % [2-(trifluoromethypimidazo[1,2-a]pyridin-3-yl]methanol). Yield: 64%.

¹H NMR δ_{H} (DMSO, ppm): 4.00 (s, 2H), 7.37 (td, J=6.8, 1.0 Hz, 1H), 7.45 (d, J=5.63 Hz, 1H), 7.52 (d, J=5.63 Hz, 1H), 7.72 (m, 1H), 7.94 (d, J=9.0 Hz, 1H), 8.93 (s, 1H), 9.94 (d, J=6.85 Hz, 1H).

### 20.2 Synthesis of [3-({[2-(trifluoromethyl)imidazo[1,2-a]pyridin-3-yl]methyl}amino)-2-thienyl]acetic acid a93

A mixture of [3-({[2-(trifluoromethyl)imidazo[1,2-a]pyridin-3-yl]methylene}amino)-2-thienyl]acetic acid a92 (0.45 g, 1.27 mmol) and Na(CN)BH₃ (0.180 g, 2.7 mmol) in absolute methanol (15 mL) is stirred for 18 h to give crude intermediate a93 (0.38 g). Yield: 84%. LC-MS (MH⁺): 356.

### 20.3 Synthesis of 4-{[2-(trifluoromethyl)imidazo[1,2-a]pyridin-3-yl]methyl}-4,6-dihydro-5H-thieno[3,2-b]pyrrol-5-one 37

DIEA (1.4 mL, 7.92 mmol) is added to a suspension of [3-({[2-(trifluoromethyl)imidazo[1,2-a]pyridin-3-yl]methyl}amino)-2-thienyl]acetic acid a93 (0.42 g, 1.32 mmol) in dichloroethane (14 mL) under stirring, and TBTU (0.466 g, 1.45 mmol) is added in 1 h. The reaction mixture is stirred overnight, and the solvents are evaporated. A 20% K₂CO₃ solution is added to the residue, and the mixture is subjected to extraction with chloroform. The combined extracts are dried over anhydrous Na₂SO₄ and evaporated.

The residue (0.61 g) is purified by chromatography on silicagel (chloroform/methanol 10/1) to afford 0.125 g of 4-{[2-(trifluoromethyl)imidazo[1,2-a]pyridin-3-yl]methyl}-4,6-dihydro-5H-thieno[3,2-b]pyrrol-5-one 37. Yield: 43%. LC-MS (MH⁺): 338.

### Example 21

### Synthesis of 4-{[2-(trifluoromethyl)imidazo[1,2-a]pyridin-3-yl]methyl}-hexahydro-5H-thieno[3,2-b]pyrrol-5-one 38

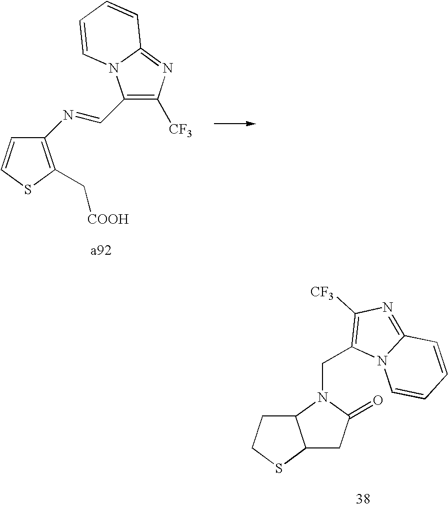

Na(CN)BH₃ (54 mg, 0.88 mol) is added to a suspension of [3-({[2-(trifluoromethyl)-imidazo[1,2-a]pyridin-3-yl]methylene}amino)-2-thienyl]acetic acid a92 (155 mg, 0.43 mmol) in acetic acid (1.7 mL) under stirring. After 5 min, a homogenous solution is formed, which is stirred at room temperature overnight. Water is added to the reaction mixture, which is evaporated to dryness, and a saturated NaHCO₃ solution (1 mL) is added to the residue.

The mixture is subjected to extraction with ethyl acetate, and the organic extract is dried over anhydrous Na₂SO₄ and evaporated. The residue (186 mg) is purified by chromatography on silicagel (CCl₄/AcOEt 1/1 v/v) to give a mixture of desired compound with [2-(trifluoromethyl)imidazo[1,2-a]pyridin-3-yl]methanol (51 mg) in a molar ratio 1/1 according to the ¹H NMR data. A second purification on silicagel (CHCl₃/MeOH 100/5 v/v) affords 35 mg of 4-{[2-(trifluoromethyl)imidazo[1,2-a]pyridin-3-yl]methyl}hexahydro-5H-thieno[3,2-b]pyrrol-5-one 38 as a white crystalline substance. Yield: 23%. LC-MS (MH⁺): 342.

### Example 22

### Synthesis of 1-(1H-imidazol-4-ylmethyl)-1H-thieno[3,4-b]pyrrol-2(3H)-one 39

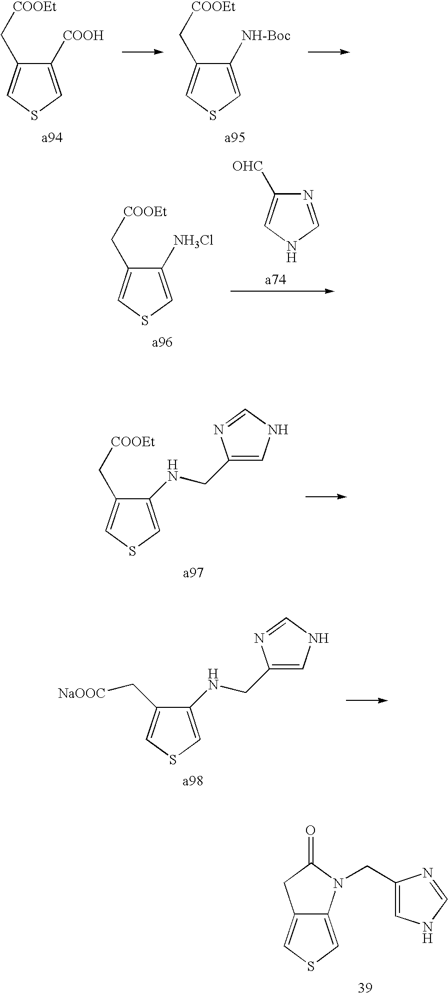

### 22.1 Synthesis of ethyl{4-[(tert-butoxycarbonyl)amino]-3-thienyl}acetate a95

Triethylamine (0.92 mL, 6.6 mmol) is added under stirring in argon to a suspension of 4-(2-ethoxy-2-oxoethyl)thiophene-3-carboxylic acid a94 (Ames D. E., Ribeiro O., Journal of the Chemical Society, Perkin Transactions 1 (1975), 14, 1390-51; 29 g, 6.0 mmol) in absolute tert-butanol (25 mL), and diphenyl azidophosphate is added to the formed homogeneous solution. The reaction mixture is stirred at room temperature for 5 min and then under reflux for 16 h. Then the mixture is cooled to room temperature and evaporated under reduced pressure. The residue is dissolved in dichloromethane (50 mL), and the solution is washed with 10% citric acid, a 10% NaHCO₃ solution and brine, dried over anhydrous Na₂SO₄, and evaporated. The oily residue (2.2 g) is purified by chromatography on silicagel (gradient CCl₄/CHCl₃ from 1/1 to 2/3 v/v) to afford ethyl{4-[(tert-butoxycarbonyl)amino]-3-thienyl}acetate a95 as a viscous oil (1.30 g). Yield: 76%.

¹H NMR δ_{H} (DMSO, ppm): 1.19 (t, J=7.1 Hz, 3H), 1.46 (s, 9H), 3.66 (s, 2H), 4.07 (q, J=7.1 Hz, 2H), 7.23 (d, J=3.4 Hz, 1H), 7.29 (s, 1H), 8.74 (s, 1H).

### 22.2 Synthesis of ethyl(4-amino-3-thienyl)acetate hydrochloride a96

4 M HCl in dioxane (8 mL, 31.8 mmol) is added under stirring in the absence of air moisture to a solution of intermediate a95 (1.30 g, 4.55 mmol) in absolute dioxane (12 mL), and the mixture stirred at room temperature for 24 h. The reaction mixture is diluted with absolute diethylether (50 mL) and stirred for 1 h. The formed precipitate is separated by filtration, washed with diethylether, and dried in a vacuum dessicator over NaOH to give ethyl(4-amino-3-thienyl)acetate hydrochloride a96 as a white powder (0.84 g). Yield: 83%.

¹H NMR δ_{H} (DMSO, ppm): 1.22 (t, J=7.1 Hz, 3H), 3.80 (s, 2H), 4.12 (q, J=7.1 Hz, 2H), 7.50 (m, 2H), 10.19 (m, 3H).

### 22.3 Synthesis of ethyl{4-[(1H-imidazol-4-ylmethyl)amino]-3-thienyl}acetate a97

A solution of 1H-imidazole-4-carbaldehyde a74 (0.613 g, 6.38 mmol) in CH₂Cl₂ (4 mL) is added under stirring in argon to a solution of ethyl(4-amino-3-thienyl)acetate hydrochloride a96 (0.707 g, 3.19 mmol) in a mixture of CH₂Cl₂ (16 mL) and TFA (16 mL). After 10 min, Et₃SiH (1.0 mL, 6.38 mmol) is added, and after 1 h Et₃SiH (0.25 mL, 1.6 mmol) is additionally added. The reaction mixture is stirred at room temperature for 18 h, and the solvents are removed under reduced pressure. The residue is treated with a 30% K2CO₃ solution, and the mixture is subjected to extraction with ethyl acetate (3×75 mL). The combined organic extracts are dried over anhydrous Na₂SO₄ and evaporated. The residue is purified by chromatography on silicagel (CHCl₃/MeOH 95/5 v/v) to give ethyl{4-[(1H-imidazol-4-ylmethyl)amino]-3-thienyl}acetate a97 as a light-yellow viscous mass (0.25 g).

Yield: 30%.

¹H NMR δ_{H} (CDCl₃, ppm): 1.24 (t, J=7.1 Hz, 3H), 3.52 (s, 2H), 4.13 (q, J=7.1 Hz, 2H), 4.29 (s, 2H), 6.05 (d, J=3.2 Hz, 1H), 7.01 (m, 2H), 7.63 (s, 1H).

### 22.4 Synthesis of sodium {4-[(1H-imidazol-4-ylmethyl)amino]-3-thienyl}acetate a98

NaOH (0.030 g, 0.70 mmol) is added under stirring in argon to a solution of ethyl{4-[(1H-imidazol-4-ylmethyl)amino]-3-thienyl}acetate a97 (0.170 g, 0.64 mmol) in ethanol (0.9 mL). The mixture is stirred at room temperature for 2.5 h, and ethanol is removed under reduced pressure. The residue is triturated with acetonitrile and twice reevaporated with acetonitrile to give crude sodium {4-[(1H-imidazol-4-ylmethyl)amino]-3-thienyl}acetate a98 (0.16 g), which is used in the next step without additional purification.

### 22.5 Synthesis of 1-(1H-imidazol-4-ylmethyl)-1H-thieno[3,4-b]pyrrol-2(3H)-one 39

TBTU (0.225 g, 0.70 mmol) is added in one portion under stirring in argon to a suspension of crude sodium salt a98 (0.16 g, 0.64 mmol) in absolute MeCN (10 mL), and DIEA (2.0 mL) is added in 15 min. The reaction mixture is additionally stirred at room temperature for 5 min and at 50-55° C. for 5 h. The mixture is cooled to room temperature, and the solvents are removed under reduced pressure. The residue (0.40 g) is purified by chromatography on silicagel (chloroform/methanol 95/5 v/v) to give 1-(1H-imidazol-4-ylmethyl)-1H-thieno[3,4-b]pyrrol-2(3H)-one 39 (0.075 g). Yield: 53%. LC-MS (MH⁺): 220.

### Example 23

### Synthesis of 2-(6-bromo-2-oxo-1,3-benzothiazol-3(2H)-yl)propanamide enantiomers 46 and 47

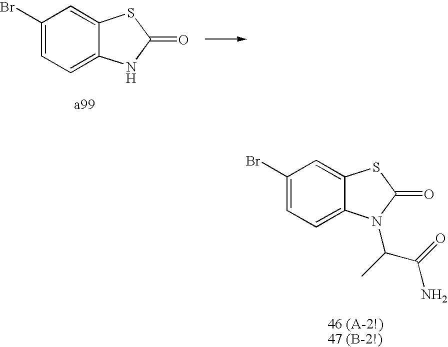

In a 100 mL three necked flask fitted with a magnetic stirrer, under inert atmosphere, 6-bromo-1,3-benzothiazol-2(3H)-one a99 (5.218 g, 22.7 mmol) is dissolved in DMF (40 mL). The solution is cooled to 0° C. and NaH (1.176 g, 29.5 mmol, 60% dispersion in oil) is carefully added. The mixture is stirred at room temperature for 20 minutes, cooled again to 0° C. and 2-bromopropionamide is added portionwise. After stirring overnight at room temperature, the mixture is poured into cold water, the solid is filtered off and washed with water and hexane. 2-(6-bromo-2-oxo-1,3-benzothiazol-3(2H)-yl)propanamide is obtained as a white solid (6.31 g). Yield: 96%. GC-MS (M³⁰ ·): 300/302.

This compound is resolved into its enantiomers by chiral chromatography (chiralcel OD 250*4.6 mm, eluent: EtOH/isohexane/DEA 50/50/0.1 v/v/v) to afford enantiomers 46 (first eluted, 1.829 g) and 47 (second eluted,1.866 g) as, after trituration in hexane, white solids.

Compound 46: Yield: 27%. GC-MS (M⁺·): 300/302.

Compound 47: Yield: 27%. GC-MS (M⁺·): 300/302.

Compounds 41 and 45 may be synthesized according to the same method.

### Example 24

### Synthesis of 2-(6-methyl-2-oxo-1,3-benzothiazol-3(2H)-yl)acetamide 49

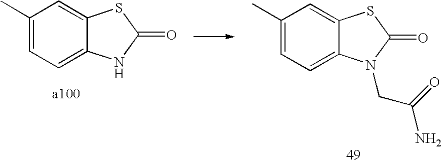

In a 50 mL three necked flask fitted with a magnetic stirrer and a reflux condenser, under inert atmosphere, KOH (0.763 g, 11.2 mmol) is added to a solution of 6-methyl-1,3-benzothiazol-2(3H)-one a100 (1.85 g, 11.2 mmol) in acetone (13 mL). The mixture is brought to reflux for 6 hours and cooled down slowly to 20° C. Distilled water (40 mL) is added, the mixture is stirred at room temperature for 30 minutes, filtered and the filtrate is washed with cold water to afford 2-(6-methyl-2-oxo-1,3-benzothiazol-3(2H)yl)acetamide 49 (2.267 g) as a white solid. Yield: 91%. GC-MS (MH⁺·): 222.

2-(6-fluoro-2-oxo-1,3-benzothiazol-3(2H)yl)acetamide 48 may be synthesized according to the same method.

### Example 25

### Synthesis of 2-(6-chloro-2-oxo-1,3-benzothiazol-3(2H)-yl)acetamide 42

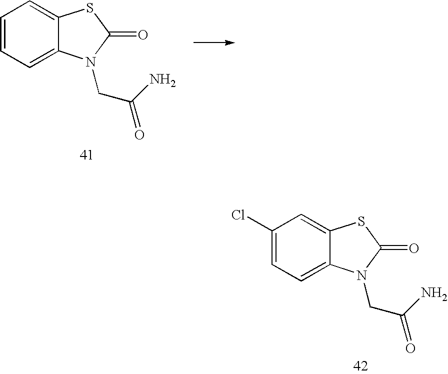

In a 100 mL flask with a magnetic stirrer, 2-(2-oxo-1,3-benzothiazol-3(2H)-yl)acetamide 41 (3.43 g, 16.5 mmol) is dissolved in 20 mL of 90% H₂SO₄. The mixture is cooled down to 0° C. and N-chlorosuccinimide (2.12 g, 16.6 mmol) is added portionwise. The mixture is stirred at room temperature for 25 minutes, poured into cold water and the solid is filtered off, washed with cold water until the pH of the mother liquid exceeds 4 and recrystallized from EtOH/H₂O (75/25 v/v) to afford 2-(6-chloro-2-oxo-1,3-benzothiazol-3(2H)-yl)acetamide 42 (1.20 g). Yield: 30%. LC-MS (MH⁺): 243/245.

Compounds 40 and 44 may be synthesized according to the same method, using N-bromosuccinimide instead of N-chlorosuccinimide.

### Example 26

### Synthesis of 6-bromo-3-(1H-imidazol-1-ylmethyl)-1,3-benzothiazol-2(3H)-one 43

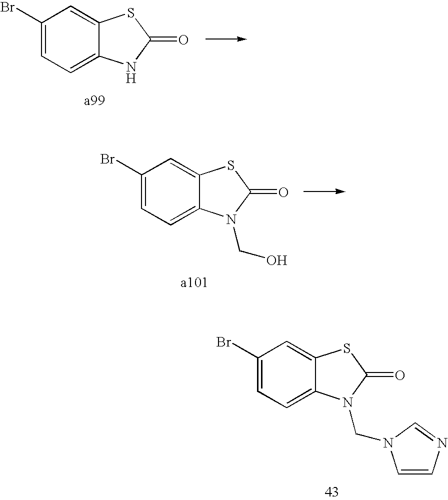

### 26.1 Synthesis of 6-bromo-3-(hydroxymethyl)-1,3-benzothiazol-2(3H)-one a101

In a 100 mL three necked flask fitted with a magnetic stirrer and a reflux condenser, 6-bromo-1,3-benzothiazol-2(3H)-one a99 (3 g, 13 mmol) and formaldehyde 35% (5 mL), are mixed in 20 mL of methanol and 10 mL of water. The mixture is brought to reflux for 4 hours and cooled down slowly to 20° C. The reaction mixture is filtered to afford 6-bromo-3-(hydroxymethyl)-1,3-benzothiazol-2(3H)-one a101 as white solid (3.37 g) which is used in the next step without any further purification. Yield: 100%. LC-MS (MH⁺): 229/231.

### 26.2 Synthesis of 6-bromo-3-(1H-imidazol-1-ylmethyl)-1,3-benzothiazol-2(3H)-one 43

In a 250 mL three necked flask fitted with a magnetic stirrer and a reflux condenser, 6-bromo-3-(hydroxymethyl)-1,3-benzothiazol-2(3H)-one a101 (2.60 g, 10 mmol) is dissolved in acetonitrile (100 mL) and 1-(1H-imidazol-1-ylcarbonyl)-1H-imidazole (2.43 g, 15 mmol) is added in one portion. The mixture is brought to reflux for one night and concentrated in vacuo to give the crude 6-bromo-3-(1H-imidazol-1-ylmethyl)-1,3-benzothiazol-2(3H)-one which is purified by chromatography on silicagel (CH₂Cl₂/EtOH/NH₄OH 95/4.5/0.5 v/v/v) to give, after recrystallization from ethanol, 6-bromo-3-(1H-imidazol-1-ylmethyl)-1,3-benzothiazol-2(3H)-one 43 as white solid (0.813 g). Yield: 26%. LC-MS (MH⁺): 310/312.

Compound 50 may be prepared according to the same method (from 6-fluoro-3-(hydroxymethyl)-1,3-benzoxazol-2(3H)-one a40).

### Example 27

### Synthesis of 1-(1H-imidazol-4-ylmethyl)pyrazolo[1,5-a]pyridin-2(1H)-one 51

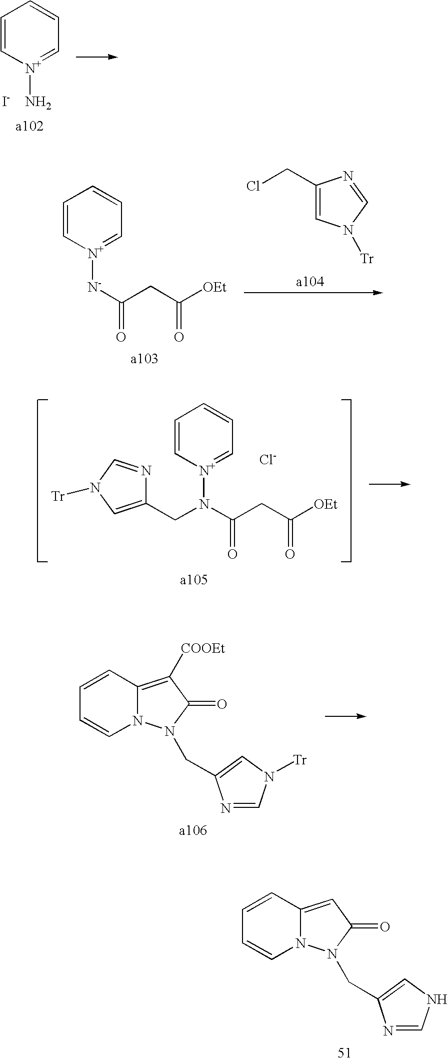

### 27.1 Synthesis of (3-ethoxy-3-oxopropanoyl)(pyridinium-1-yl)azanide a103

A mixture of 1-aminopyridinium iodide a102 (3.0 g, 13.5 mmol), diethyl malonate (18 mL, 119 mmol) and anhydrous K₂CO₃ (22.5 g, 163 mmol) in absolute ethanol (225 mL) is stirred at room temperature for 3 days. The reaction mixture is filtered and the filtrate is evaporated. The residue is purified by chromatography on basic aluminum oxide (gradient 1: ether/chloroform from 100/0 to 0/100 v/v; gradient 2: chloroform/methanol from 20/0 to 20/1 v/v). According to the ¹H NMR data, the obtained fraction (2.68 g) is the mixture of intermediate a103 with ethyl malonate in a molar ratio 1:1. This fraction is washed several times with hexane until disappearance of ethylmalonate to afford pure (3-ethoxy-3-oxopropanoyl)(pyridinium-1-yl)azanide a103 (1.41 g). Yield: 49%.

¹H NMR δ_{H} (DMSO, ppm): 5.11 (s, 1H), 5.21 (s, 2H), 6.52 (m, 1H), 7.02 (s, 1H), 7.15 (m, 2H), 7.56 (s, 1H), 8.48 (d, J=6.5 Hz, 1H), 12.02 (m, 1H).

### 27.2 Synthesis of ethyl 2-oxo-1-[(1-trityl-1H-imidazol-4-yl)methyl]-1,2-dihydropyrazolo[1,5-a]pyridine-3-carboxylate-methanol (1:2) a106

A mixture of (3-ethoxy-3-oxopropanoyl)(pyridinium-1-yl)azanide a103 (0.52 g, 2.48 mmol) and 4-(chloromethyl)-1-trityl-1H-imidazole a104 (0.83 g, 2.31 mmol) in dry acetone (9 mL) is refluxed under argon for 4 days. Acetone is removed under reduced pressure, and the residue, which contains not isolated intermediate 1-{(3-ethoxy-3-oxopropanoyl)[(1-trityl-1H-imidazol-4-yl)methyl]amino}pyridinium chloride a105, is dissolved in absolute ethanol (9 mL). Anhydrous K₂CO₃ (0.8 g, 5.80 mmol) is added, and the mixture is stirred under reflux for 42 h. The reaction mixture is cooled to room temperature, filtered and evaporated. The residue (1.5 g) is purified by chromatography on silicagel (gradient CHCl₃/MeOH from 50/1 to 10/1 v/v) to afford 0.170 g of ethyl 2-oxo-1-[(1-trityl-1H-imidazol-4-yl)methyl]-1,2-dihydropyrazolo[1,5-a]pyridine-3-carboxylate-methanol (1:2) a106. Yield: 14%.

¹H NMR δ_{H} (DMSO, ppm): 1.26 (t, J=7.1 Hz, 3H), 4.18 (q, J=7.1 Hz, 2H), 5.24 (s, 2H), 7.00 (m, 8H), 7.37 (m, 10H), 7.61 (m, 1H), 7.88 (d, J=8.6 Hz, 1H), 8.61 (d, J=6.8 Hz, 1H).

### 27.3 Synthesis of 1-(1H-imidazol-4-ylmethyl)pyrazolo[1,5-a]pyridin-2(1H)-one 51

A mixture of intermediate a106 (0.43 g, 0.81 mmol) and 7 N HCl (7 mL) is stirred under reflux for 13 h. The reaction mixture is cooled to room temperature, and the formed precipitate is separated by filtration and washed with 2 N HCl. The filtrate is neutralized with solid K₂CO₃ and subjected to extraction with the mixture tert-butanol/chloroform 1/1 (v/v) (3×30 mL). The combined extracts are dried over anhydrous Na₂SO₄. The solvents are removed under reduced pressure, and the residue is purified by chromatography on silica gel (AcOEt/MeOH 1/1 v/v), then by chromatography on basic aluminum oxide (gradient chloroform/methanol from 40/0 to 40/1 v/v) to give 1-(1H-imidazol-4-ylmethyl)pyrazolo[1,5-a]pyridin-2(1H)-one 51 (0.034 g). Yield: 20%. LC-MS (MH⁺): 215.

### Example 28

### Synthesis of 2-(6-chloro-3-oxo-3,4-dihydroisoquinolin-2(1H)-yl)propanamide 52

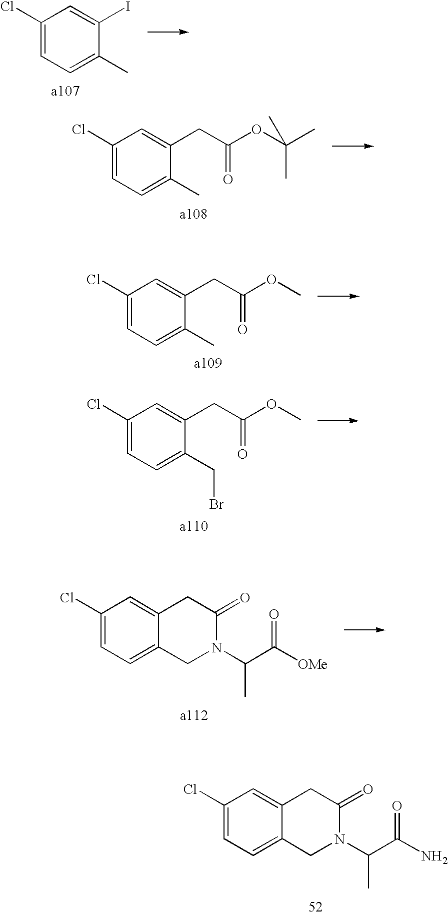

### 28.1 Synthesis of tert-butyl(5-chloro-2-methylphenyl)acetate a108

A solution of P(t-Bu)₃ (0.0757 M, 5.19 mL, 0.393 mmol) and a solution of 4-chloro-2-iodo-1-methylbenzene a107 (5.0 g, 19.8 mmol) and tert-butyl acetate (2.53 g, 21.8 mmol) in degassed toluene (50 mL) are added to a mixture of LiHMDS (7.60 g, 45.5 mmol) and Pd(dba)₂ (0.226 g, 0.393 mmol), under argon atmosphere. The reaction mixture is stirred at room temperature for 20 hours (according to the LC-MS data, 2-3 hours are sufficient for the reaction to go to completion) and it is quenched with a saturated ammonium chloride solution (50 mL). After 15 min the organic layer is separated, and the aqueous layer is extracted with diethylether. The combined organic extracts are dried over anhydrous Na₂SO₄ and evaporated. The residue is purified by chromatography on silicagel (petroleum ether/AcOEt 10/1 v/v) to afford, after evaporation, tert-butyl(5-chloro-2-methylphenyl)acetate a108 (3.70 g) as a yellow oil (90% purity). Yield: 77%.

¹H NMR δ_{H} (CDCl₃, 400 MHz, ppm): 1.44 (s, 9H); 2.26 (s, 3H); 3.50 (s, 2H), 7.06-7.19 (m, 3H).

### 28.2 Synthesis of methyl(5-chloro-2-methylphenyl)acetate a109

HCl (2.3 M in methanol, 33.3 mL, 76.5 mmol) is added to tert-butyl(5-chloro-2-methylphenyl)acetate a108 (3.70 g, 15.3 mmol), and the reaction mixture is stirred at 50-55° C. for 20 hours. The reaction mixture is concentrated under reduced pressure, the residue is dissolved in ethyl acetate, washed with a saturated solution of NaHCO₃, dried over anhydrous Na₂SO₄, and the solution is concentrated to give 2.43 g of methyl(5-chloro-2-methylphenyl)acetate a109 (85% purity). Yield: 80%.

¹H NMR δ_{H} (CDCl₃, ppm): 2.27 (s, 3H), 3.60 (s, 2H), 3.70 (s, 3,H), 7.09-7.23 (m, 3H).

### 28.3 Synthesis of methyl[2-(bromomethyl)-5-chlorophenyl]acetate a110

To a solution of methyl(5-chloro-2-methylphenyl)acetate a109 (2.40 g, 12.1 mmol) in dry benzene (5 mL), benzoyl peroxide (0.025 g, 0.1 mmol) is added and the mixture is heated to intense reflux. A mixture of NBS (2.05 g, 11.5 mmol) and benzoyl peroxide (0.025 g, 0.1 mmol) is added portionwise over 20-25 minutes. The reaction mixture is brought to reflux for another 40 min, and it is cooled down to room temperature and diluted with an equal volume of hexane. The precipitate is filtered off, washed with hexane, with hexane/ether mixture (1/1 v/v) and once again with hexane. The filtrates are combined and concentrated. To give a mixture of methyl[2-(bromomethyl)-5-chlorophenyl]acetate a110 (3.37 g) with starting compound in 66:17 ratio (according to GC-MS data), which is used in the next step without further purification. GC-MS (M⁺·): 277.

¹H NMR δ_{H} (CDCl₃, ppm): 3.72 (s, 3H), 3.77 (s, 2H), 4.53 (s, 2H), 7.25-7.32 (m, 3H).

### 28.4 Synthesis of methyl 2-(6-chloro-3-oxo-3,4-dihydroisoquinolin-2(1H)-yl)propanoate a111

To a stirred suspension of methyl alaninate hydrochloride (1.48 g, 10.6 mmol) in absolute acetonitrile (15 mL), iPr₂NEt (3.5 mL, 20.2 mmol) is added. After 5 min the precipitate is dissolved, and a clear solution is formed, to which a solution of crude methyl[2-(bromomethyl)-5-chlorophenyl]acetate a110 (1.65 g, 5.9 mmol) in absolute acetonitrile (5 mL) is added. The reaction mixture is stirred at 75-80° C. for 25 hours, and for another 20 hours at room temperature. The solvents are removed under reduced pressure, and the residue is dissolved with 100 mL of methylene chloride. The solution is washed with a 10% solution of NaHSO₄ (2×25 mL), dried over anhydrous Na₂SO₄, and the solvent is removed under reduced pressure. The residue (1.87 g) is purified by chromatography silicagel (petroleum ether/AcOEt 3/2 v/v) to afford methyl 2-(6-chloro-3-oxo-3,4-dihydroisoquinolin-2(1H)-yl)propanoate a111 (0.683 g). Yield: 43% (for 2 steps).

¹H NMR δ_{H} (CDCl₃, ppm): 1.47 (d, J=7.33 Hz, 3H), 3.62 (s, 2H), 3.71 (s, 3H); 4.43 (dd, J=29.84 Hz, J=15.40 Hz, 2H); 5.35 (q, J=7.33 Hz, 1H), 7.11-7.23 (m, 3H).

### 28.5 Synthesis of 2-(6-chloro-3-oxo-3,4-dihydroisoquinolin-2(1H)-yl)propanamide 52

Methyl 2-(6-chloro-3-oxo-3,4-dihydroisoquinolin-2(1H)-yl)propanoate a111 (0.683 g, 2.55 mmol) is dissolved in 5 mL of saturated ammonia in methanol and the reaction mixture is allowed to stand at room temperature. After 5 days, another 5 mL of saturated methanolic ammonia are added. After 2 more days the solvents are removed under reduced pressure. The residue (0.618 g) is washed with hexane and dried in vacuum to afford 0.552 g of 2-(6-chloro-3-oxo-3,4-dihydroisoquinolin-2(1H)-yl)propanamide 52.

Yield: 85%. LC-MS (MH⁺): 253/255.

### Example 29

### Synthesis of 5-chloro-2-(1H-imidazol-4-ylmethyl)-1,4-dihydroisoquinolin-3(2H)-one 53

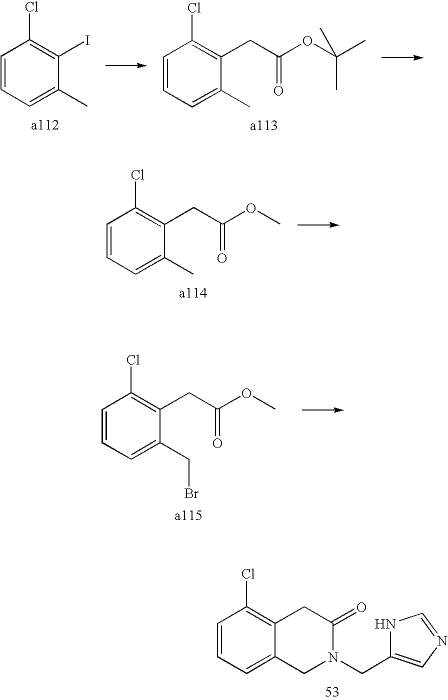

### 29.1 Synthesis of tert-butyl(2-chloro-6-methylphenyl)acetate a113

A 0.0757 M solution of P(t-Bu)₃ in toluene (5.19 mL, 0.393 mmol), a solution of 3-chloro-2-iodotoluene a112 (5.0 g, 19.8 mmol) in degassed toluene (10 mL) and a solution of tert-butylacetate (2.53 g, 21.8 mmol) in degassed toluene (10 mL) are sequentially added to a mixture of LiHMDS (7.60 g, 45.5 mmol) and Pd(dba)₂ (0.226 g, 0.393 mmol) in degassed toluene (35 mL) under stirring in argon. The reaction mixture is stirred at room temperature for 2 h and decomposed by the addition of a saturated NH₄Cl solution (50 mL). After 15 min, the organic layer is separated, and the aqueous layer is subjected to extraction with diethylether. The combined organic extracts are dried over anhydrous Na₂SO₄ and evaporated. The residue (6.38 g) is purified by chromatography on silicagel (petroleum ether/AcOEt 10/1 v/v). The solvents are removed to afford tert-butyl(2-chloro-6-methylphenyl)acetate a113 as a light-yellow oil (4.80 g). Yield: 100%.

GC-MS (M⁺·−(C₄H₈)): 184.

### 29.2 Synthesis of methyl(2-chloro-6-methylphenyl)acetate a114

2.6 M HCl in methanol (30 mL, 78 mmol, 3.9 eq) is added to tert-butyl(2-chloro-6-methyl-phenyl)acetate a113 (4.80 g, 19.8 mmol). The reaction mixture is stirred at 50-55° C. for 4 h and evaporated under reduced pressure. The residue is dissolved in ethyl acetate. The solution is washed with a saturated NaHCO₃ solution, dried over anhydrous Na₂SO₄, and evaporated to give methyl(2-chloro-6-methylphenyl)acetate a114 as a light-yellow oil (3.20 g). Yield: 81%.

¹H NMR δ_{H} (DMSO, ppm): 2.29 (s, 3H), 3.63 (s, 3H), 3.86 (s, 2H), 7.18 (m, 2H), 7.29 (m, 1H).

### 29.3 Synthesis of methyl[2-(bromomethyl)-6-chlorophenyl]acetate a115

Benzoyl peroxide (30 mg) is added to a solution of methyl(2-chloro-6-methylphenyl)acetate a114 (3.20 g, 16.1 mmol) in dry benzene 10 mL, and the mixture is refluxed intensively. The mixture of NBS (2.72 g, 15.3 mmol) and benzoyl peroxide (35 mg) is added in portions under stirring for 35 min. The reaction mixture is refluxed additionally for 1 h, cooled to room temperature, and diluted with hexane (20 mL). The formed precipitate is separated by filtration, washed with hexane, a 1/1 hexane-ether mixture and hexane again. The filtrates are combined and evaporated to give the mixture (4.3 g, 96%) of methyl[2-(bromomethyl)-6-chlorophenyl]acetate a115 with starting compound a114 in the molar ratio 2:1 according to the ¹H NMR data. The mixture is used as such in the next step. GC-MS (M⁺·): 278.

### 29.4 Synthesis of 5-chloro-2-(1H-imidazol-4-ylmethyl)-1,4-dihydroisoquinolin-3(2H)-one 53

To the previous mixture containing intermediate a115 (0.44 g, 1.15 mmol, 73 wt %) is added (1H-imidazol-4-ylmethyl)amine dihydrochloride (0.59 g, 3.46 mmol, 3 eq) [from A. Turner et al., J. Am. Chem. Soc. (1949), 71, 2801-2803.], and anhydrous cesium carbonate (1.88 g, 5.76 mmol, 5 eq) in dry DMF (15 mL). The mixture is vigorously stirred at 60° C. for 3 h. The reaction mixture is cooled, concentrated under reduced pressure, diluted with water, and subjected to extraction with ethyl acetate (three times). The combined organic extracts are washed with brine, dried over anhydrous Na₂SO₄, and evaporated. The residue (0.35 g) is purified by chromatography on silicagel (gradient CHCl₃/MeOH from 20/1 to 10/1 v/v) to afford 5-chloro-2-(1H-imidazol-4-ylmethyl)-1,4-dihydroisoquinolin-3(2H)-one 53 (0.15 g).

Yield: 50%. LC-MS (MH⁺): 262/264.

### Example 30

### Synthesis of 2-(6-chloro-2-oxo-3,4-dihydroquinolin-1(2H)-yl)acetamide 54

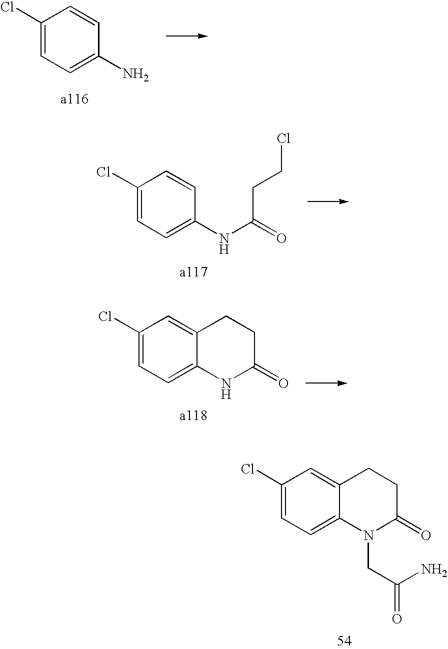

### 30.1 Synthesis of 3-chloro-N-(4-chlorophenyl)propanamide a117

In a tree-necked flask fitted with a magnetic stirrer and a reflux condenser, under nitrogen atmosphere, 4-chloroaniline a116 (25.52 g, 200 mmol) is dissolved in acetone (40 mL). A solution of 3-chloropropanoyl chloride (10 mL, 100 mmol) in acetone (20 mL) is added and after 0.5 h the reaction mixture is heated to reflux for 1.5 h. The reaction mixture is cooled down to room temperature, quenched with HCl (5 N, 100 mL), filtered and concentrated. The crude reaction mixture is recrystallized from MeOH/H₂O (200 mL, 1/1 v/v) to afford 3-chloro-N-(4-chlorophenyl)propanamide a117 (20.21 g). Yield: 46%. GC-MS (M⁺·): 217/219.

### 30.2 Synthesis of 6-chloro-3,4-dihydro-2(1H)-quinolinone a118

In a tree-necked flask fitted with a mechanical stirrer, under nitrogen atmosphere, 3-chloro-N-(4-chlorophenyl)propanamide a117 (10.9 g, 50 mmol) is heated to 140° C. At this temperature, AlCl₃ (13.34 g, 100 mmol) is carefully added and the reaction mixture is stirred at 140° C. for 24 h. The reaction mixture is cooled down to 0° C., HCl/H₂O (100 mL, 10/90 w/w) is carefully added, H₂O (200 mL) is added and the reaction mixture is filtered.

The obtained off-white solid is washed with H₂O and with hexane. The crude residue is then dissolved in acetone/EtOH at room temperature, cooled down to 0° C., filtered on celite and the filtrate is concentrated to afford 6-chloro-3,4-dihydro-2(1H)-quinolinone a118 (5 g). Yield: 55%. GC-MS (M⁺·): 181/183.

### 30.3 Synthesis of 2-(6-chloro-2-oxo-3,4-dihydroquinolin-1(2H)-yl)acetamide 54

In a tree-necked flask fitted with a magnetic stirrer, under nitrogen atmosphere, at room temperature, 6-chloro-3,4-dihydro-2(1H)-quinolinone a118 (2.5 g, 13.7 mmol) is dissolved in DMF (75 mL). NaH (60% dispersion in mineral oil; 0.606 g, 15.14 mmol) is added and the heterogenous reaction mixture is stirred at room temperature for 0.5 h. 2-bromoacetamide (2.28 g, 16.52 mmol) is then added, the clear solution is stirred at room temperature for 1 h, quenched with saturated NH₄Cl and water and extracted with ethyl acetate (3 times). The organic phases are washed with H₂O, brine, dried over MgSO₄ and concentrated. The crude reaction mixture is triturated in Et₂O and recrystallized from EtOH to afford 2-(6-chloro-2-oxo-3,4-dihydroquinolin-1(2H)-yl)acetamide 54 (0.620 g, 2.6 mmol).

Yield: 19%

GC-MS (M⁺·): 238/240.

Compounds 55, 57 and 58 may be synthesized according to the same method.

### Example 31

### Synthesis of 1-(1H-imidazol-4-ylmethyl)-3,4-dihydroquinolin-2(1H)-one 56

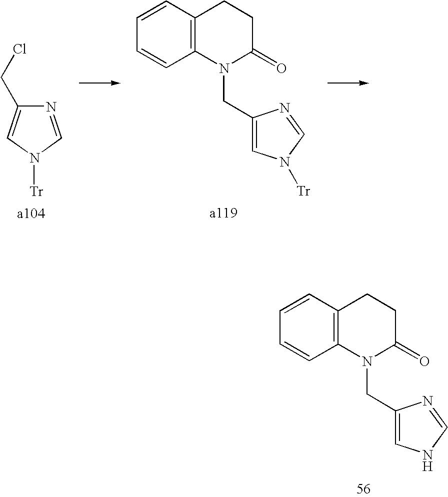

### 31.1 Synthesis of 1-[(1-trityl-1H-imidazol-4-yl)methyl]-3,4-dihydro-2(1H)-quinolinone a119

In a 50 mL three necked flask fitted with a magnetic stirrer and a reflux condenser, 3,4-dihydro-2(1H)-quinolinone a104 (1.25 g, 8.5 mmol) is dissolved in DMF (20 mL). NaH (60% dispersion in mineral oil; 0.44 g, 11.05 mmol) is added and the mixture is heated at 60° C. After 1 h 4-(chloromethyl)-1-trityl-1H-imidazole B (3.66 g, 10 mmol) is added and the reaction mixture is further heated for 2 h. The reaction mixture is cooled down to room temperature, quenched with a saturated solution of NH₄Cl and concentrated under reduced pressure. The crude reaction mixture is purified by chromatography on silicagel (eluent: EtOAc/MeOH 96/4 v/v) to afford 1-[(1-trityl-1H-imidazol-4-yl)methyl]-3,4-dihydro-2(1H)-quinolinone a119, which is used without further purification in the next step. Yield: 100%.

LC-MS (MH⁺): 470.

### 31.2 Synthesis of 1-(1H-imidazol-4-ylmethyl)-3,4-dihydro-2(1H)-quinolinone hydrochloride 56

In a 100 mL, three necked flask fitted with a magnetic stirrer, 1-[(1-trityl-1H-imidazol-4-yl)methyl]-3,4-dihydro-2(1H)-quinolinone a119 (8.5 mmol) is dissolved in 2 N HCl (50 mL) and heated at 100° C. for 1 h. The mixture is cooled down to room temperature, filtered and the filtrate is concentrated to dryness by azeotropy with toluene. The obtained crude reaction mixture is purified by chromatography on silicagel (eluent: CH₂Cl₂/MeOH/NH₄OH 8/1.8/0.2 v/v/v), treated with a solution of HCl (2.2 N in Et₂O; 2.5 mL), filtered and recrystallized from EtOH/Et₂O to afford 1-(1H-imidazol-4-ylmethyl)-3,4-dihydro-2(1H)-quinolinone hydrochloride 56 (0.89 g). Yield: 40%. LC-MS (MH⁺): 228

### Example 32

### Synthesis of 7-chloro-2-(1H-imidazol-4-ylmethyl)-1,2,4,5-tetrahydro-3H-2-benzazepin-3-one 60

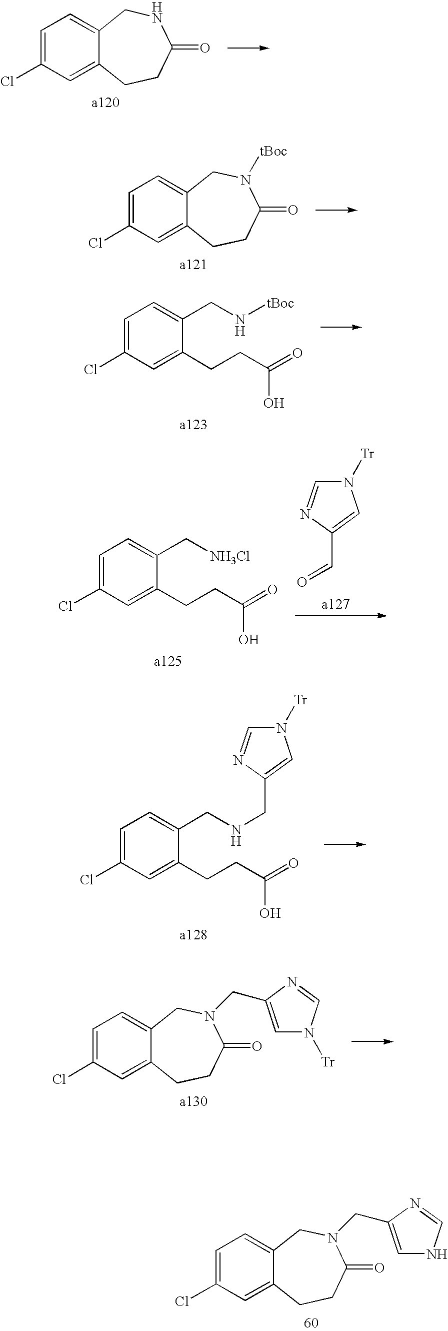

### 32.1 Synthesis of tert-butyl 7-chloro-3-oxo-1,3,4,5-tetrahydro-2H-2-benzazepine-2-carboxylate a121

Boc₂O (1.34 g, 6.2 mmol), Et₃N (0.43 mL, 3.1 mmol), and DMAP (0.38 g, 3.1 mmol) are sequentially added under stirring in argon to a solution of 7-chloro-1,2,4,5-tetrahydro-3H-2-benzazepin-3-one a120 (0.60 g, 3.1 mmol) in dry dichloromethane (18 mL). The mixture is stirred at 40° C. for 18 h and poured into a saturated solution of CuSO₄ (10 mL). The organic layer is separated, and the aqueous layer is extracted with ethyl acetate. The combined organic phases are dried over anhydrous Na₂SO₄, and the solvents are removed under reduced pressure. The residue (0.96 g) is purified by chromatography on silicagel (hexane/AcOEt 2/1 v/v) to afford tert-butyl 7-chloro-3-oxo-1,3,4,5-tetrahydro-2H-2-benzazepine-2-carboxylate a121 (0.72 g). Yield: 75%.

¹H NMR δ_{H} (DMSO, ppm): 1.41 (s, 9H), 3.01 (m, 2H), 3.13 (m, 2H), 4.91 (s, 2H), 7.21-7.23 (m, 2H), 7.26 (s, 1H).

Tert-butyl 7-chloro-2-oxo-1,2,4,5-tetrahydro-3H-3-benzazepine-3-carboxylate a122 may be synthesized according to the same method.

¹H NMR δ_{H} (DMSO, ppm): 1.41 (s, 9H), 3.01 (m, 2H), 3.13 (m, 2H), 4.91 (s, 2H), 7.21-7.23 (m, 2H), 7.26 (s, 1H).

### 32.2 Synthesis of 3-(2-{[(tert-butoxycarbonyl)amino]methyl}-5-chlorophenyl)propanoic acid a123

A 1 N LiOH solution (7.0 mL, 7.0 mmol) is added dropwise under stirring to a solution of tert-butyl 7-chloro-3-oxo-1,3,4,5-tetrahydro-2H-2-benzazepine-2-carboxylate a121 (0.69 g, 2.33 mmol) in THF (12 mL). In 2 h, THF is evaporated under reduced pressure. The aqueous residue is acidified with 10% AcOH to pH 6, and the reaction mixture is extracted with ether (3×30 mL). The combined organic extracts are dried over anhydrous Na₂SO₄, and the solvent is removed under reduced pressure to give 3-(2-{[(tert-butoxycarbonyl)amino]-methyl}-5-chlorophenyl)propanoic acid a123 (0.80 g). Yield: 100%.

¹H NMR δ_{H} (DMSO, ppm): 1.38 (s, 9H), 2.48 (m, 2H+solvent peak), 2.83 (t, J=7.6 Hz, 2H), 4.13 (d, J=6.1 Hz, 2H), 7.22 (d, J=2.2 Hz, 3H), 7.34 (m, 1H).

(2-{2-[(tert-butoxycarbonyl)amino]ethyl}-4-chlorophenyl)acetic acid a124 may be synthesized according to the same method and is used as such in the next step.

### 32.3 Synthesis of 3-[2-(aminomethyl)-5-chlorophenyl]propanoic acid hydrochloride a125

4 N HCl in dioxane (6.0 mL, 24 mmol) is added under stirring to a solution of 3-(2-{[(tert-butoxycarbonyl)amino]methyl}-5-chlorophenyl)propanoic acid a123 (0.78 g, 2.48 mmol) in absolute dioxane (10 mL). The mixture is stirred at room temperature overnight. Dioxane is removed under reduced pressure, and the residue is washed with diethylether, filtered, and vacuum-dried to give 3-[2-(aminomethyl)-5-chlorophenyl]propanoic acid hydrochloride a125 (0.60 g). Yield: 97%.

¹H NMR δ_{H} (DMSO, ppm): 2.57 (t, J=7.8 Hz, 2H), 2.88 (t, J=7.6 Hz, 2H), 3.57 (s, 4H), 4.05 (s, 2H), 7.35 (m, 2H), 7.48 (m, 1H).

[2-(2-aminoethyl)-4-chlorophenyl]acetic acid hydrochloride a126 may be synthesized according to the same method.

¹H NMR δ_{H} (DMSO, ppm): 2.90 (m, 2H), 2.98 (m, 2H), 3.68 (s, 2H), 7.27 (m, 2H), 7.33 (s, 1H), 8.10 (m, 3H).

### 32.4 Synthesis of 345-chloro-2-({[(1-trityl-1H-imidazol-4-yl)methyl]amino}methyl)phenyl]propanoic acid a128

3-[2-(aminomethyl)-5-chlorophenyl]propanoic acid hydrochloride a125 (0.54 g, 2.16 mmol), Et₃N (1.8 mL, 13.0 mmol), and trimethylorthoformate (0.30 mL, 2.16 mmol) are added under stirring and prevention from entrance of air moisture to a solution of 1-trityl-1H-imidazole-4-carbaldehyde (0.73 g, 2.16 mmol) in absolute methanol (76 mL), and the mixture is stirred at room temperature for 16 h. NaBH₄ (0.13 g, 3.45 mmol) is added, and the mixture is stirred for 1 h and quenched by the addition of a saturated NaHCO₃ solution to pH 5-6. The solvents are removed under reduced pressure, and the obtained mass is subjected to extraction with ethyl acetate. Water is added to the residue, and undissolved residue is separated by filtration and dried to give 3-[5-chloro-2-({[(1-trityl-1H-imidazol-4-yl)methyl]amino}methyl)phenyl]propanoic acid a128 (0.71 g). The organic extract is dried over anhydrous Na₂SO₄ and evaporated to give additionally intermediate a128 (0.40 g).

The total yield of intermediate a128 is 1.11 g. Yield: 96%.

¹H NMR δ_{H} (DMSO, ppm): 2.48 (m, 2H), 2.81 (t, J=7.6 Hz, 2H), 3.86 (s, 2H), 3.93 (s, 2H), 6.96 (s, 1H), 7.10 (m, 6H), 7.20-7.48 (m, 13H).

[4-chloro-2-(2-{[(1-trityl-1H-imidazol-4-yl)methyl]amino}ethyl)phenyl]acetic acid a129 may be synthesized according to the same method.

¹H NMR δ_{H} (DMSO, ppm): (m, 2H), 3.06 (m, 2H), 3.65 (s, 2H), 4.07 (s, 2H), 7.10 (m, 7H), 7.28 (m, 3H), 7.41 (m, 9H), 7.51 (s, 1H).

### 32.5 Synthesis of 7-chloro-2-[(1-trityl-1H-imidazol-4-yl)methyl]-1,2,4,5-tetrahydro-3H-2-benzazepin-3-one a130

DIEA (2.2 mL, 12.42 mmol) is added under stirring in argon to a solution of 345-chloro-2-({[(1-trityl-1H-imidazol-4-yl)methyl]amino}methyl)phenyl]propanoic acid a128 (1.11 g, 2.07 mmol) in dry dichloroethane (290 mL). The mixture is stirred for 20 min, 2-(1H-benzotriazol-1-yl)-1,1,3,3-tetramethyluronium tetrafluoroborate (TBTU, 0.73 g, 2.28 mmol) is added, and the mixture is stirred at room temperature overnight. A saturated NaHCO₃ solution is added, and the organic layer is separated. The aqueous layer is subjected to extraction with dichloromethane, and the combined organic extracts are dried over anhydrous Na₂SO₄. The organic solvents are removed under reduced pressure, and the residue (1.65 g) is purified by chromatography on silicagel (chloroform/methanol 50:1) to give intermediate a130 (0.92 g). Yield: 85.5%.

¹H NMR δ_{H} (DMSO, ppm): 2.80 (m, 2H), 2.95 (m, 2H), 4.42 (s, 2H), 4.56 (s, 2H), 5.75 (m, 2H), 6.43 (s, 1H), 6.94 (m, 8H), 7.16 (d, J=1.90 Hz, 1H), 7.20 (d, J=1.23 Hz, 1H), 7.37 (m, 9H).

7-chloro-3-[(1-trityl-1H-imidazol-4-yl)methyl]-1,3,4,5-tetrahydro-2H-3-benzazepin-2-one a131 may be synthesized according to the same method.

¹H NMR δ_{H} (DMSO, ppm): 2.84 (t, J=5.4 Hz, 2H), 3.76 (m, 2H), 3.83 (s, 2H), 4.40 (s, 2H), 6.70 (d, J=1.0 Hz, 1H), 7.05 (m, 7H), 7.15 (s, 2H), 7.32 (d, J=1.2 Hz, 1H), 7.37 (m, 9H)

### 32.6 Synthesis of 7-chloro-2-(1H-imidazol-4-ylmethyl)-1,2,4,5-tetrahydro-3H-2-benzazepin-3-one hydrochloride 60

4 N HCl in dioxane (18.0 mL, 72 mmol) is added under stirring and prevention from entrance of air moisture to a solution of intermediate a130 (0.68 g, 1.30 mmol) in absolute dioxane (8.5 mL), and the mixture is stirred at room temperature for 52 h. The formed precipitate is separated by filtration, washed three times with diethylether, and vacuum-dried to give 7-chloro-2-(1H-imidazol-4-ylmethyl)-1,2,4,5-tetrahydro-3H-2-benzazepin-3-one hydrochloride 60 as a white powder (0.247 g). Yield: 61%.

¹H NMR δ_{H} (DMSO, ppm): 2.87 (m, 2H), 3.08 (m, 2H), 4.64 (d, J=16.31 Hz, 4H), 7.15 (s, 2H), 7.24 (s, 1H), 7.46 (s, 1H), 8.97 (d, J=1.00 Hz, 1H), 14.48 (s, 1H).

Compound 61 may be prepared according to the same method.

### Example 33

### Synthesis of 7-chloro-2-{[2-(trifluoromethyl)imidazo[1,2-a]pyridin-3-yl]methyl}-1,2,4,5-tetrahydro-3H-2-benzazepin-3-one 59

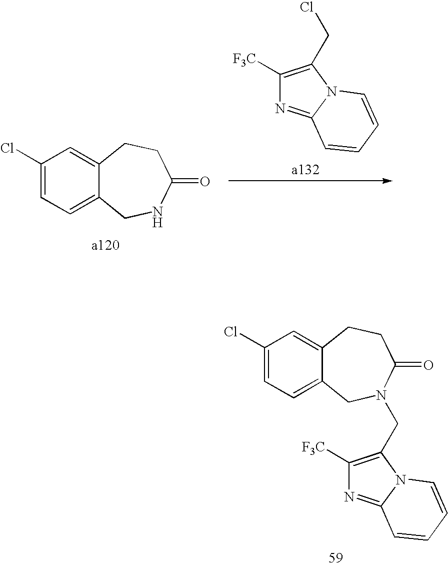

### 33.1 Synthesis of 3-(chloromethyl)-2-(trifluoromethyl)imidazo[1,2-a]pyridine a132

3-(chloromethyl)-2-(trifluoromethyl)imidazo[1,2-a]pyridine a132 is prepared immediately prior to the synthesis from its hydrochloride (0.15 g, 0.55 mmol) [synthesized according to the methods described by S. Mavel et al in Bioorg. Med. Chem. (2002), 10, 941-946 or by J. J. Kaminski, A. M. Doweyko in J. Med. Chem. (1997), 40, 427-436].

A suspension of 3-(chloromethyl)-2-(trifluoromethyl)imidazo[1,2-a]pyridine hydrochloride (0.15 g, 0.55 mmol) in dry diethylether (5 mL) is treated with iPrEt₂N (0.10 mL, 0.58 mmol) under stirring for 5 min, filtered, and evaporated. The residue is vacuum-dried for 1 h to give 3-(chloromethyl)-2-(trifluoromethyl)imidazo[1,2-a]pyridine a132 (0.127 g). Yield: 98%.

### 33.2 Synthesis of 7-chloro-2-{[2-(trifluoromethyl)imidazo[1,2-a]pyridin-3-yl]methyl}-1,2,4,5-tetrahydro-3H-2-benzazepin-3-one 59

n-BuLi (1.6 M in hexane; 0.35 mL, 0.56 mmol) is added under stirring in argon at −78° C. to a solution of 7-chloro-1,2,4,5-tetrahydro-3H-2-benzazepin-3-one a120 (0.108 g, 0.55 mmol) in absolute THF (15 mL). The mixture is stirred for 0.5 h, and a solution of 3-(chloromethyl)-2-(trifluoromethyl)imidazo[1,2-a]pyridine a132 (0.130 g, 0.55 mmol) in absolute THF (5 mL) is added dropwise. The reaction mixture is stirred at −70 to −50° C. for 1 h, at room temperature for 1 h, and at 60° C. for 16 h. The mixture is cooled to room temperature, and a saturated NH₄Cl solution (10 mL) is added. The organic layer is separated, and the aqueous layer is extracted with dichloromethane. The combined organic extracts are dried over anhydrous Na₂SO₄, and the solvents are removed under reduced pressure. The residue is washed 5 times with dry diethylether and vacuum-dried to give 7-chloro-2-{[2-(trifluoromethyl)imidazo-[1,2-a]pyridin-3-yl]methyl}-1,2,4,5-tetrahydro-3H-2-benzazepin-3-one 59 (0.120 g).

Yield: 55%. LC-MS (MH⁺): 394.

Compound 62 may be prepared according to the same method.

### Example 34

### Synthesis of 2-[(2E)-2-(cyanoimino)-4-propylpyrrolidin-1-yl]butanamide 68

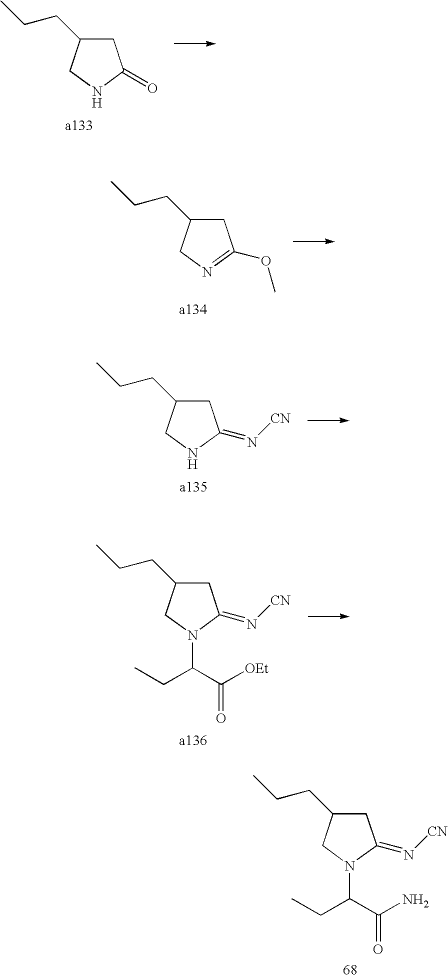

### 34.1 Synthesis of 5-methoxy-3-propyl-3,4-dihydro-2H-pyrrole a134

A solution of trimethyloxonium borofluoride (11.2 g, 76 mmol) in dry CH₂Cl₂ (60 mL) is added to a solution of 4-propylpyrrolidin-2-one a133 (8.0 g, 63 mmol) in dry CH₂Cl₂ (60 mL). The reaction mixture is stirred at room temperature for 16 h, diluted with CH₂Cl₂ (150 mL), washed with 5% NaHCO₃ and water, dried with anhydrous Na₂SO₄, and evaporated in vacuum. The residue is dissolved in dry chloroform, passed through a layer of silica gel, and eluted thrice with chloroform. The combined filtrates are evaporated in vacuum to afford 5-methoxy-3-propyl-3,4-dihydro-2H-pyrrole a134 (7.27 g). Yield: 82%.

¹H NMR δ_{H} (DMSO, ppm): 0.87 (t, J=6.8 Hz, 3H), 1.20-1.38 (m, 4H), 2.08 (dd, J=6.87 and 16.1 Hz, 1H), 2.30-2.40 (m, 1H), 2.50-5.57 (m, 1H overlap with solvent), 3.11 (dt, J=5.87 and 13.7 Hz, 1H), 3.64 (dd, J=7.8 and 13.7 Hz, 1H), 3.67 (s, 3H).

### 34.2 Synthesis of [4-propylpyrrolidin-2-ylidene]cyanamide a135

5-methoxy-3-propyl-3,4-dihydro-2H-pyrrole a134 (4.0 g, 28 mmol) is dissolved in methanol (60 mL). A 50% solution of cyanamide (2.62 g, 2.43 mL, 31 mmol) in water is added and the reaction mixture is stirred at room temperature for 2 h. The solvents are evaporated in vacuum. The residue is dried in a vacuum desiccator over anhydrous CaCl2 to afford 4.28 g of [4-propylpyrrolidin-2-ylidene]cyanamide a135. Yield: 99%.

LC-MS (MH⁺): 152.

### 34.3 Synthesis of ethyl 2-[(2E)-2-(cyanoimino)-4-propylpyrrolidin-1-yl]butanoate a136

A 60% suspension of NaH in mineral oil (67 mg, 1.67 mmol) is added in portions to a stirred solution of [4-propylpyrrolidin-2-ylidene]cyanamide a135 (230 mg, 1.52 mmol) in absolute DMF (4 mL), under argon. The reaction mixture is stirred at room temperature for 30 min., then ethyl 2-bromobutanoate (311 mg, 236 μL, 1.60 mmol) is added dropwise. The reaction mixture is stirred for 3 h, then decomposed with brine. The resulting mixture is extracted three times with ethyl acetate. The combined organic layers are washed with brine and dried over anhydrous Na₂SO₄. The solvents are removed under reduced pressure. The residue (490 mg) is purified by chromatography on silicagel (eluent: hexane/ethyl acetate 2:1) to afford 0.30 g of ethyl 2-[(2E)-2-(cyanoimino)-4-propylpyrrolidin-1-yl]butanoate a136. Yield: 74%.

LC-MS (MH⁺): 266.

[(2E)-1-(1H-imidazol-4-ylmethyl)-4-propylpyrrolidin-2-ylidene]cyanamide 65 may be synthesized according to the same method.

### 34.4 Synthesis of 2-[(2E)-2-(cyanoimino)-4-propylpyrrolidin-1-yl]butanamide 68

Ethyl 2-[(2E)-2-(cyanoimino)-4-propylpyrrolidin-1-yl]butanoate a136 (250 mg, 0.94 mmol) is dissolved in a 7 M methanol solution of ammonia (30 mL). The reaction mixture is stirred at room temperature for 10 h, and evaporated to dryness under reduced pressure. The residue is triturated with a 1:1 ether/pentane mixture, filtered off, and dried to afford 190 mg of 2-[(2E)-2-(cyanoimino)-4-propylpyrrolidin-1-yl]butanamide 68. Yield: 86%. LC-MS (MH⁺): 237.

Compounds 66 and 67 may be synthesized according to the same method.

### Example 35

### Synthesis of N-[(2E)-1-(1H-imidazol-4-ylmethyl)-4-propylpyrrolidin-2-ylidene]methanesulfonamide 69

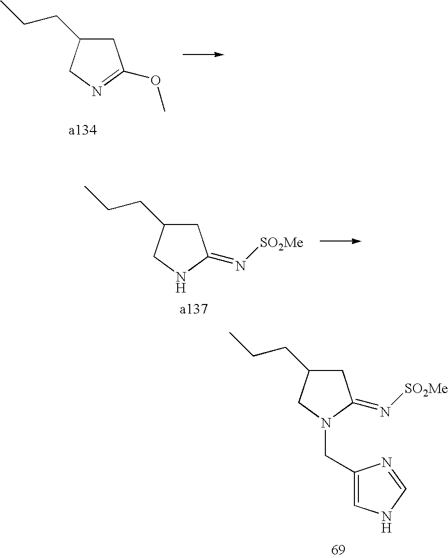

### 35.1 Synthesis of N-[(2E)-4-propylpyrrolidin-2-ylidene]methanesulfonamide a137

A mixture of 5-methoxy-3-propyl-3,4-dihydro-2H-pyrrole a134 (300 mg, 2.13 mmol) and methanesulfonamide (134 mg, 1.42 mmol) in absolute methanol (5 mL) is refluxed for 3 days under anhydrous conditions. The reaction mixture is cooled to room temperature and evaporated to dryness under reduced pressure. The residue is purified by chromatography on silicagel (eluent: hexane/ethyl acetate 1:1) to afford 240 mg of crude N-[(2E)-4-propylpyrrolidin-2-ylidene]methanesulfonamide a137 as a colorless powder which is used for the next step without any further purification. Yield: 83%.

LC-MS (MH⁺): 205.

### 35.2 Synthesis of N-[(2E)-1-(1H-imidazol-4-ylmethyl)-4-propylpyrrolidin-2-ylidene]methanesulfonamide 69

N-[(2E)-1-(1H-imidazol-4-ylmethyl)-4-propylpyrrolidin-2-ylidene]methanesulfonamide 69 may be synthesised from N-[(2E)-4-propylpyrrolidin-2-ylidene]methanesulfonamide a137, according to the method described in example 34.3.

LC-MS (MH⁺): 285.

Table I indicates the IUPAC name of the compound, the ion peak observed in mass spectroscopy and the ¹H NMR description.

TABLE I
Physical Characterization of Example Compounds.

| | | MH⁺ | ¹H NMR δ_{H} (400 MHZ, DMSO unless |
|---|---|---|---|
| n^{o} | IUPAC Name | (M^{+.}) | otherwise specified) |
| 1 | (2S)-2-[3-(4-nitrophenyl)-5- | (292) | (250 MHz): 0.90 (t, J = 8.1 Hz, 3 H), |
| | oxoimidazolidin-1-yl]butanamide | | 1.65-2.0 (m, 2 H), 4.1 (s, 2 H), 4.5 (m, 1 H), |
| | | | 4.90 (d, J = 2.9 Hz, 1 H), 5.05 (d, J = 2.9 Hz, 1 |
| | | | H), 6.75 (d, J = 7.4 Hz, 2 H), 7.10 (s, 1 H), |
| | | | 7.55 (s, 1 H), 8.15 (d, J = 7.4 Hz, 2 H) |
| 2 | (2S)-2-[3-(2,4-dinitrophenyl)-5- | 338 | (250 MHz): 0.85 (t, J = 7.5 Hz, 3 H), |
| | oxoimidazolidin-1-yl]butanamide | | 1.65-2.0 (m, 2 H), 4.0 (s, 2 H), 4.5 (m, 1 H), |
| | | | 4.9 (d, J = 3.7 Hz, 1 H), 5.10 (d, J = 43.7 Hz, 1 |
| | | | H), 7.15 (s, 1 H), 7.25 (d, J = 8.2 Hz, 1 H), |
| | | | 7.65 (s, 1 H), 8.3 (m, 1 H), 8.15 (s, 1 H) |
| 3 | (2S)-2-(5-oxo-3-phenylimidazolidin- | (247) | (250 MHz): 0.85 (t, J = 7.4 Hz, 3 H), |
| | 1-yl)butanamide | | 1.6-2.0 (m, 2 H), 3.9 (s, 2 H), 4.5 (m, 1 H), 4.75 (d, J = 2.9 Hz, |
| | | | 1 H), 4.90 (d, J = 2.9 Hz, 1 H), |
| | | | 6.65 (d, J = 8.0 Hz, 2 H), 6.75 (t, J = 8.0 Hz, |
| | | | 1 H), 7.1 (s, 1 H), 7.25 (t, J = 8.0 Hz, 2 H), |
| | | | 7.6 (s, 1 H) |
| 4 | 2-[5-(iodomethyl)-2-oxo-1,3- | 313 | (250 MHz): 0.9 (t, J = 8.0 Hz, 3 H), 1.6 (m, 1 |
| | oxazolidin-3-yl]butanamide | | H), 1.85 (m, 1 H), 3.13 (m, 1 H), 3.50 (m, 2 |
| | | | H), 3.85 (t, J = 6.4 Hz, 1 H), 4.05 (m, 1 H), |
| | | | 4.57 (m, 1 H), 7.03 (s, 1 H), 7.50 (s, 1 H) |
| 5 | 2-(2-oxo-2,5-dihydro-1H-pyrrol-1- | (168) | 0.80 (t, 3 H, 6.2 Hz), 1.5-2.0 (m, 2 H), 4.0 (d, |
| | yl)butanamide | | J = 20.8 Hz, 1 H), 4.3 (d, J = 20.8 Hz, 1 H), |
| | | | 4.45 (m, 1 H), 6.1 (m, 1 H), 6.95 (s, 1 H), |
| | | | 7.3 (m, 1 H), 7.45 (s, 1 H) |
| 6 | 2-(2-oxo-4-phenyl-2,5-dihydro-1H- | 245 | (250 MHz): 0.85 (t, J = 7.8 Hz, 3 H), |
| | pyrrol-1-yl)butanamide | | 1.7-2.2 (m, 2 H), 4.40-4.75 (m, 3 H), 6.6 (s, 1 H), |
| | | | 7.0 (s, 1 H), 7.40 (m, 4H), 7.7 (m, 2 H) |
| 7 | 2-(4-methyl-2-oxo-2,5-dihydro-1H- | 191 | (250 MHz): 0.85 (t, J = 5.9 Hz, 3 H), |
| | pyrrol-1-yl)butanamide | | 1.5-1.95 (m, 2 H), 2.1 (s, 3 H), 3.9 (d, J = 18.4 Hz, |
| | | | 1 H), 4.2 (d, J = 18.4 Hz, 1 H), 4.4 (m, 1 |
| | | | H), 5.8 (s, 1 H) 6.95 (s, 1 H), 7.4 (s, 1 H) |
| 8 | (+)-(2S)-2-(2-oxo-4-propyl-2,5- | 211 | (250 MHz): 0.95 (m, 6H), 1.5-2.1 (m, 4H), |
| | dihydro-1H-pyrrol-1-yl)butanamide | | 2.35 (m, 2 H), 3.85 (d, J = 25.1 Hz, 1 H), |
| | | | 4.05 (d, J = 25.1 Hz, 1 H), 4.50 (m, 1 H), |
| | | | 5.5 (s, 1 H), 5.85 (s, 1 H), 6.4 (s, 1 H) |
| 9 | (2S)-2-(2-oxo-5-propyl-1,3-thiazol- | 229 | 0.78 (t, J = 7.3 Hz, 3 H), 0.89 (t, 3 H, J = 7.3 Hz), |
| | 3(2H)-yl)butanamide | | 1.47-1.52 (m, 2 H), 1.76-1.94 (m, 2 H), |
| | | | 2.46 (t, J = 7.3 Hz, 2 H), 4.6 (dd, J = 10.1 Hz, |
| | | | J = 5.6 Hz, 1 H), 6.82 (s, 1 H), 7.23 (s, 1 |
| | | | H), 7.67 (s, 1 H) |
| 10 | 2-(2-oxo-5-propyl-1,3-thiazol- | 215 | 0.90 (t, J = 7.3 Hz, 3 H), 1.43-1.52 (m, 5H), |
| | 3(2H)-yl)propanamide | | 2.45 (m, 2 H), 4.74 (q, J = 7.3 Hz, 1 H), |
| | | | 6.81 (s, 1 H), 7.19 (s, 1 H), 7.59 (s, 1 H) |
| 11 | 2-(5-butyl-2-oxo-1,3-thiazolidin-3- | 245 | (CDCl₃): 0.85-1.1 (m, 6 H), 1.35 (m, 4 H), |
| | yl)butanamide | | 1.58-1.93 (m, 3 H), 1.97 (m, 1 H), |
| | | | 3.26-3.36 (m, 1 H), 3.64-3.79 (m, 2 H), 4.48 (m, 1 H), |
| | | | 5.41 (s, 1 H), 6.05 (s, 1 H) |
| 12 | 2-(5-butyl-2-oxo-1,3-thiazolidin-3- | 231 | (CDCl₃): 0.92 (m, 3 H), 1.29-1.43 (m, 6 H), |
| | yl)propanamide | | 1.64 (s, 1 H), 1.74 (m, 2 H), 3.31 (m, 1 H), |
| | | | 3.72 (m, 2 H), 4.75 (m, 1 H), 5.42 (s, 1 H), |
| | | | 6.05 (s, 1 H) |
| 13 | 2-(2-oxo-5-phenyl-1,3-thiazolidin-3- | 251 | 1.34 (dd, J = 14.1, 7.3 Hz, 3 H), 3.79 (m, 1 |
| | yl)propanamide | | H), 4.09 (m, 1 H), 4.57 (m, 1 H), 5.09 (dt, J = 18.2, |
| | | | 7.3 Hz, 1 H), 7.13 (d, J = 17.4 Hz, 1 |
| | | | H), 7.29-7.44 (m, 3 H), 7.45-7.58 (m, 3 H) |
| 14 | 2-(2-oxo-5-propyl-1,3-thiazolidin-3- | 231 | 0.84 (t, J = 7.3 Hz, 3 H), 0.90 (t, J = 7.1 Hz, |
| | yl)butanamide | | 3 H), 1.35 (m, 2 H), 1.63 (m, 2 H), 1.73 (m, |
| | | | 1 H), 1.83 (m, 1 H), 3.26 (dd, J = 10.4, 6.3 Hz, |
| | | | 1 H), 3.81 (m, 1 H), 3.94 (dd, J = 10.1 Hz, |
| | | | J = 7.1 Hz, 1 H), 4.33 (dd, J = 10.4, 5.3 Hz, |
| | | | 1 H), 7.11 (s, 1 H), 7.50 (s, 1 H) |
| 15 | 2-(2-oxo-5-phenyl-1,3-thiazolidin-3- | 265 | 0.79 (t, J = 7.1 Hz, 3 H), 0.89 (t, J = 7.1 Hz, |
| | yl)butanamide | | 3 H), 1.5-1.85 (m, 4H), 3.54 (m, 1 H), |
| | | | 3.97 (m, 2 H), 4.22 (m, 1 H), 4.41 (m, 2 H), |
| | | | 5.06 (m, 2 H), 7.14 (m, 2 H), 7.30-7.61 (m, 12 H) |
| 16 | 2-(2-oxo-5-propyl-1,3-thiazolidin-3- | 217 | 0.91 (t, J = 7.3 Hz, 3 H), 1.29 (d, J = 7.3 Hz, |
| | yl)propanamide | | 3 H), 1.36 (dd, J = 14.9, J = 7.3 Hz, 2 H), |
| | | | 1.52-1.88 (m, 2 H), 3.32-3.50 (m, 1 H), |
| | | | 3.66-3.96 (m, 2 H), 4.48 (m, 1 H), 7.09 (s, 1 |
| | | | H), 7.43 (s, 1 H) |
| 17 | (2S)-2-[2-oxo-5-(2,2,2- | 271 | 0.84 (t, J = 7.3 Hz, 3 H), 1.59-2.88 (m, 2 H), |
| | trifluoroethyl)-1,3-thiazolidin-3- | | 2.77-2.99 (m, 2 H), 3.37 (dd, J = 10.4, J = 7.3 Hz, |
| | yl]butanamide | | 2 H), 3.61-3.79 (m, 1 H), 4.34 (m, 1 |
| | | | H), 7.13 (s, 1 H), 7.55 (s, 1 H) |
| 18 | 1-{[6-chloro-2- | 333/ | (CDCl₃): 1.78 (s, 4 H), 2.42 (s, 2 H), 3.27 (s, |
| | (trifluoromethyl)imidazo[1,2- | 335 | 2 H), 5.12 (s, 2 H), 7.19 (d, J = 9.5 Hz, 1 |
| | b]pyridazin-3-yl]methyl}piperidin-2- | | H), 7.97 (d, J = 9.5 Hz, 1 H) |
| | one | | |
| 19 | 1-(1H-imidazol-4-ylmethyl)-5- | 222 | 0.86 (t, J = 6.8 Hz, 3 H), 1.18-1.44 (m, 5 H), |
| | propylpiperidin-2-one hydrochloride | | 1.79 (m, 2 H), 2.29 (m, 2 H), 2.94 (m, 1 H), |
| | | | 3.44 (m, 1 H), 4.52 (m, 2 H), 7.55 (s, 1 H), |
| | | | 9.01 (s, 1 H), 14.42 (s, 1 H) |
| 20 | 1-(1H-imidazol-1-ylmethyl)-5- | 222 | 0.85 (t, J = 7.0 Hz, 3 H), 1.16-1.38 (m, 5 H), |
| | propylpiperidin-2-one | | 1.74 (m, 2 H), 2.30 (m, 2 H), 2.89 (m, 1 H), |
| | | | 3.39 (ddd, J = 11.5, 4.8, 1.5 Hz, 1 H), |
| | | | 5.34 (d, J = 13.6 Hz, 1 H), 5.52 (d, J = 13.6 Hz, 1 |
| | | | H), 6.90 (s, 1 H), 7.17 (s, 1 H), 7.71 (s, 1 H) |
| 21 | 1-(imidazo[1,2-a]pyridin-3- | 272 | 0.83 (t, J = 7.0 Hz, 3 H), 1.13-1.42 (m, 5 H), |
| | ylmethyl)-5-propylpiperidin-2-one | | 1.77 (m, 2 H), 2.33 (m, 2 H), 2.90 (m, 1 H), |
| | trifluoroacetate | | 3.33 (m, 1 H), 4.96 (s, 2 H), 7.57 (td, J = 6.5, |
| | | | 1.5 Hz, 1 H), 7.98 (m, 2 H), 8.25 (s, 1 |
| | | | H), 8.89 (d, J = 7.0 Hz, 1 H) |
| 22 | 1-(1H-imidazol-1-ylmethyl)-5- | 256 | 1.90 (m, 1 H), 2.01 (m, 1 H), 2.42 (dd, J = 6.3, |
| | phenylpiperidin-2-one | | 3.8 Hz, 1 H), 3.09 (m, 1 H), 3.32 (m, 2 |
| | | | H), 3.55 (ddd, J = 11.5, 5.0, 1.5 Hz, 1 H), |
| | | | 5.39 (m, 1 H), 5.56 (m, 1 H), 6.90 (s, 1 H), |
| | | | 7.20 (s, 1 H), 7.29 (m, 5 H), 7.71 (s, 1 H) |
| 23 | 1-(imidazo[1,2-a]pyridin-3- | 306 | 1.88 (m, 1 H), 1.95-2.05 (m, 1 H), |
| | ylmethyl)-5-phenylpiperidin-2-one | | 2.40-2.48 (m, 2 H), 3.03 (m, 1 H), 3.22 (m, 1 H), |
| | | | 3.40 (m, 1 H), 4.91 (q, J = 15.3 Hz, 2 H), 6.96 (t, |
| | | | J = 6.8 Hz, 1 H), 7.23 (m, 6 H), 7.58 (m, 2 |
| | | | H), 8.56 (d, J = 6.8 Hz, 1 H) |
| 24 | 1-(imidazo[1,2-a]pyridin-3- | 306 | 1.95 (m, 2 H), 2.56 (d, J = 4.3 Hz, 2 H), |
| | ylmethyl)-4-phenylpiperidin-2-one | | 3.10 (m, 1 H), 3.37 (m, 2 H), 5.03 (dd, J = 52.5 Hz, |
| | hydrochloride | | J = 15.8 Hz, 2 H), 7.22 (m, 3 H), |
| | | | 7.29 (m, 2 H), 7.61 (td, J = 6.8, 1.5 Hz, 1 H), |
| | | | 8.01 (m, 2 H), 8.30 (s, 1 H), 8.94 (d, J = 7.0 Hz, 1 |
| | | | H) |
| 25 | 1-(1H-imidazol-1-ylmethyl)-4- | 256 | 2.62 (m, 2 H), 3.20 (m, 1 H), 3.71-3.81 (m, 1 |
| | phenylpiperidin-2-one | | H), 4.01 (m, 1 H), 4.16 (dd, J = 7.5, 4.3 Hz, |
| | hydrochloride | | 2 H), 6.35 (m, 2 H), 7.86 (m, 3 H), 7.95 (m, |
| | | | 2 H), 8.36 (d, J = 13.3 Hz, 2 H), 9.86 (s, 1 |
| | | | H) |
| 26 | 1-(imidazo[1,2-a]pyridin-3- | 272 | (CDCl₃): 0.89 (t, J = 6.5 Hz, 3 H), |
| | ylmethyl)-4-propylpiperidin-2-one | | 1.23-1.45 (m, 5 H), 1.81 (m, 1 H), 1.93 (m, 1 H), |
| | hydrochloride | | 2.03 (dd, J = 17.6, 10.8 Hz, 1 H), 2.58 (ddd, J = 17.6, |
| | | | 4.8, 1.5 Hz, 1 H), 3.24-3.33 (m, 1 H), |
| | | | 3.35-3.42 (m, 1 H), 4.90 (d, J = 15.8 Hz, 1 |
| | | | H), 5.19 (d, J = 15.8 Hz, 1 H), 7.35 (t, J = 7.0 Hz, |
| | | | 1 H), 7.82 (m, 1 H), 8.13 (s, 1 H), |
| | | | 8.29 (d, J = 9.0 Hz, 1 H), 9.09 (d, J = 7.0 Hz, |
| | | | 1 H) |
| 27 | 1-(1H-imidazol-5-ylmethyl)-4- | 222 | 0.87 (t, J = 7.0 Hz, 3 H), 1.19-1.26 (m, 2 H), |
| | propylpiperidin-2-one | | 1.27-1.33 (m, 2 H), 1.36-1.45 (m, 1 H), |
| | trifluoroacetate | | 1.71-1.97 (m, 3 H), 2.36 (ddd, J = 16.8, 4.5, 1.5 Hz, |
| | | | 1 H), 3.27 (m, 2 H), 4.53 (s, 2 H), |
| | | | 7.55 (s, 1 H), 8.99 (s, 1 H), 14.39 (m, 1 H) |
| 28 | 1-(1H-imidazol-1-ylmethyl)-4- | 222 | 0.84 (t, J = 7.0 Hz, 3 H), 1.14-1.41 (m, 5 H), |
| | propylpiperidin-2-one | | 1.71 (m, 1 H), 1.83 (m, 1 H), 1.94 (dd, J = 17.3, |
| | | | 10.8 Hz, 1 H), 2.37 (ddd, J = 17.3, 5.0, |
| | | | 2.0 Hz, 1 H), 3.23 (ddd, J = 11 Hz, J = 11 Hz, |
| | | | J = 4.8 Hz, 1 H), 3.36 (ddd, J = 12.0, 5.5, |
| | | | 3.5 Hz, 1 H), 5.39 (d, J = 13.6 Hz, 1 H), |
| | | | 5.49 (d, J = 13.6 Hz, 1 H), 6.91 (s, 1 H), 7.17 (s, |
| | | | 1 H), 7.71 (s, 1 H) |
| 29 | 1-{[6-chloro-2- | 347/ | (CDCl₃): 1.39 (m, 2 H), 1.67 (m, 4 H), |
| | (trifluoromethyl)imidazo[1,2- | 349 | 2.57 (m, 2 H), 3.38 (m, 2 H), 5.12 (s, 2 H), |
| | b]pyridazin-3-yl]methyl}azepan-2- | | 7.20 (d, J = 9.5 Hz, 1 H), 7.98 (d, J = 9.5 Hz, 1 |
| | one | | H) |
| 30 | 1-(1H-imidazol-5-ylmethyl)-5- | 236 | 0.83 (t, J = 7.0 Hz, 3 H), 0.92 (m, 1 H), |
| | propylazepan-2-one | | 1.04 (m, 1 H), 1.13 (m, 2 H), 1.25 (m, 2 H), |
| | | | 1.46 (m, 1 H), 1.70 (m, 2 H), 2.31 (dd, J = 13.6, |
| | | | 7.5 Hz, 1 H), 2.54 (m, 1 H), 3.37 (m, 2 H), |
| | | | 4.31 (m, 1 H), 4.42 (m, 1 H), 6.88 (s, 1 H), |
| | | | 7.53 (s, 1 H), 11.87 (m, 1 H) |
| 31 | 5-propyl-1-{[2- | 354 | 0.44 (m, 1 H), 0.77 (t, J = 7.0 Hz, 3 H), |
| | (trifluoromethyl)imidazo[1,2- | | 0.99 (m, 3 H), 1.14 (m, 2 H), 1.48 (m, 2 H), |
| | a]pyridin-3-yl]methyl}azepan-2-one | | 1.75 (m, 1 H), 2.42 (dd, J = 13.6, 7.5 Hz, 1 H), |
| | | | 2.64 (t, J = 13.1 Hz, 1 H), 3.09 (dd, J = 15.3, |
| | | | 6.3 Hz, 1 H), 3.42 (dd, J = 15.3, 10.5 Hz, 1 |
| | | | H), 5.01 (dd, J = 57 Hz, J = 15.8 Hz, 2 H), |
| | | | 7.17 (td, J = 6.8, 0.8 Hz, 1 H), 7.49 (ddd, J = 8.8 Hz, |
| | | | J = 6.5 Hz, J = 0.8 Hz, 1 H), |
| | | | 7.73 (d, J = 9.3 Hz, 1 H), 8.44 (d, J = 7.0 Hz, 1 H) |
| | 1-(1H-imidazol-5-ylmethyl)-5- | 270 | 1.44 (m, 1 H), 1.58 (q, J = 12.8 Hz, 1 H), |
| | phenylazepan-2-one | | 1.80 (m, 2 H), 2.40 (dd, J = 13.6, 7.0 Hz, 1 |
| | | | H), 2.75 (t, J = 12.5 Hz, 2 H), 3.40 (dd, J = 15.3, |
| | | | 5.5 Hz, 1 H), 3.55 (dd, J = 15.3, 10.5 Hz, |
| | | | 1 H), 4.37 (d, J = 14.8 Hz, 1 H), 4.51 (d, |
| | | | J = 14.8 Hz, 1 H), 6.94 (s, 1 H), 7.17 (m, 3 |
| | | | H), 7.27 (m, 2 H), 7.56 (s, 1 H) |
| 32 | 5-phenyl-1-{[2- | 388 | 0.90 (q, J = 12.0 Hz, 1 H), 1.49 (q, J = 12.5 Hz, |
| | (trifluoromethyl)imidazo[1,2- | | 1 H), 1.59-1.68 (m, 1 H), 1.81-1.91 (m, 1 |
| | a]pyridin-3-yl]methyl}azepan-2-one | | H), 2.49 (m, 1 H), 2.73 (m, 1 H), 2.85 (t, J = 12.8 Hz, |
| | | | 1 H), 3.13 (dd, J = 15.3, 5.8 Hz, 1 |
| | | | H), 3.61 (dd, J = 15.3, 10.8 Hz, 1 H), |
| | | | 5.02 (d, J = 15.8 Hz, 1 H), 5.17 (d, J = 15.8 Hz, 1 |
| | | | H), 6.96 (d, J = 7.3 Hz, 2 H), 7.14 (m, 1 H), |
| | | | 7.22 (m, 3 H), 7.53 (m, 1 H), 7.75 (d, J = 9.3 Hz, |
| | | | 1 H), 8.49 (d, J = 7.0 Hz, 1 H) |
| 33 | 1-(1H-imidazol-5-ylmethyl)-6- | 236 | 0.85 (t, J = 7.0 Hz, 3 H), 1.19 (m, 3 H), |
| | propylazepan-2-one | | 1.29 (m, 3 H), 1.44-1.75 (m, 3 H), 2.32 (m, 1 H), |
| | | | 2.43 (dd, J = 13.6, 9.8 Hz, 1 H), 4.37 (dd, J = 33.1 Hz, |
| | | | J = 14.6 Hz, 2 H), 6.91 (s, 1 H), |
| | | | 7.58 (s, 1 H) |
| 34 | 1-(1H-imidazol-4-ylmethyl)-4- | 236 | 0.78 (t, J = 7.3 Hz, 3 H), 0.96-1.10 (m, 2 H), |
| | propylazepan-2-one | | 1.10-1.28 (m, 4 H), 1.39 (m, 1 H), |
| | | | 1.65-1.80 (m, 2 H), 2.24-2.37 (m, 1 H), 2.46-2.62 (m, 1 |
| | | | H), 3.10-3.26 (m, 2 H), 4.26 (d, J = 14.8 Hz, |
| | | | 1 H), 4.49 (d, J = 14.6 Hz, 1 H), 6.92 (s, 1 |
| | | | H), 7.57 (s, 1 H) |
| 35 | 4-(1H-imidazol-4-ylmethyl)-4,6- | 220 | 3.61 (s, 2 H), 4.69 (s, 2 H), 6.88 (d, J = 5.0 Hz, |
| | dihydro-5H-thieno[3,2-b]pyrrol-5- | | 1 H), 7.03 (s, 1 H), 7.40 (d, J = 5.0 Hz, 1 |
| | one | | H), 7.54 (s, 1 H), 11.95 (m, 1 H) |
| 36 | 2-(5-oxo-5,6-dihydro-4H- | 197 | (CDCl3): 3.68 (s, 2 H), 4.35 (s, 2 H), 5.38 (s, |
| | thieno[3,2-b]pyrrol-4-yl)acetamide | | 1 H), 5.93 (s, 1 H), 6.81 (d, J = 5.3 Hz, 1 H) |
| | | | and 7.2 (d, J = 5.0 Hz, 1 H) |
| 37 | 4-{[2-(trifluoromethyl)imidazo[1,2- | 338 | 3.73 (s, 2 H), 5.36 (s, 2 H), 6.58 (d, J = 5.0 Hz, |
| | a]pyridin-3-yl]methyl}-4,6-dihydro- | | 1 H), 7.17 (t, J = 6.8 Hz, 1 H), 7.49 (m, 2 |
| | 5H-thieno[3,2-b]pyrrol-5-one | | H), 7.73 (d, J = 9.0 Hz, 1 H), 8.50 (d, J = 7.0 Hz, |
| | | | 1 H) |
| 38 | 4-{[2-(trifluoromethyl)imidazo[1,2- | 342 | 1.74-1.88 (m, 1 H), 2.23 (m, 1 H), 2.39 (dd, |
| | a]pyridin-3-yl]methyl}hexahydro- | | J = 17.6, 2.0 Hz, 1 H), 2.55 (m, 1 H), |
| | 5H-thieno[3,2-b]pyrrol-5-one | | 2.69-2.77 (m, 1 H), 2.86 (m, 1 H), 3.88 (m, 1 H), |
| | | | 4.09 (m, 1 H), 4.76 (d, J = 16.3 Hz, 1 H), |
| | | | 5.14 (d, J = 16.3 Hz, 1 H), 7.16 (td, J = 6.8, |
| | | | 0.8 Hz, 1 H), 7.49 (m, 1 H), 7.72 (d, J = 9.3 Hz, |
| | | | 1 H), 8.51 (d, J = 7.0 Hz, 1 H) |
| 39 | 1-(1H-imidazol-4-ylmethyl)-1H- | 220 | 3.43 (s, 2 H), 4.66 (s, 2 H), 6.50 (d, J = 2.3 Hz, |
| | thieno[3,4-b]pyrrol-2(3H)-one | | 1 H), 7.14 (d, J = 44.7 Hz, 2 H), 7.57 (m, |
| | | | 1 H) |
| 40 | 2-(6-bromo-2-oxo-1,3- | 287/ | — |
| | benzothiazol-3(2H)-yl)acetamide | 289 | |
| 41 | 2-(2-oxo-1,3-benzothiazol-3(2H)- | (208) | — |
| | yl)acetamide | | |
| 42 | 2-(6-chloro-2-oxo-1,3-benzothiazol- | 243/ | 4.52 (s, 2 H), 7.15 (d, J = 8.6 Hz, 1 H), |
| | 3(2H)-yl)acetamide | 245 | 7.29 (s, 1 H), 7.36 (m, 1 H), 7.68 (s, 1 H), 7.79 (s, |
| | | | 1 H) |
| 43 | 6-bromo-3-(1H-imidazol-1- | 310/ | 6.15 (s, 2 H), 6.87 (s, 1 H), 7.22 (s, 1 H), |
| | ylmethyl)-1,3-benzothiazol-2(3H)- | 312 | 7.53-7.96 (m, 4H) |
| | one | | |
| 44 | 6-bromo-3-(2-oxopropyl)-1,3- | (285/ | — |
| | benzothiazol-2(3H)-one | 287) | |
| 45 | 2-(6-nitro-2-oxo-1,3-benzothiazol- | 253 | — |
| | 3(2H)-yl)acetamide | | |
| 46 | 2-(6-bromo-2-oxo-1,3- | (300/ | 1.5 (d, J = 7.2 Hz, 3 H), 5.17 (q, J = 7.2 Hz, |
| | benzothiazol-3(2H)-yl)propanamide | 302) | 1 H), 7.13 (d, J = 8.9 Hz, 1 H), 7.32 (s, 1 H), |
| | | | 7.51 (dd, J = 8.7 Hz, J = 2.1 Hz, 1 H), |
| | | | 7.59 (s, 1 H), 7.96 (d, J = 2.1 Hz, 1 H) |
| 47 | 2-(6-bromo-2-oxo-1,3- | (300/ | 1.5 (d, J = 7.3 Hz, 3 H), 5.17 (q, J = 7.3 Hz, |
| | benzothiazol-3(2H)-yl)propanamide | 302) | 1 H), 7.13 (d, J = 8.7 Hz, 1 H), 7.32 (s, 1 H), |
| | | | 7.51 (dd, J = 8.7 Hz, J = 2.1 Hz, 1 H), |
| | | | 7.58 (s, 1 H), 7.96 (d, J = 2.1 Hz, 1 H) |
| 48 | 2-(6-fluoro-2-oxo-1,3-benzothiazol- | 284 | 4.55 (s, 2 H), 7.12-7.25 (m, 1 H), 7.32 (s, 1 |
| | 3(2H)-yl)acetamide | | H), 7.61-7.69 (m, 2 H), 7.73 (s, 1 H) |
| 49 | 2-(6-methyl-2-oxo-1,3- | (222) | 2.28 (s, 3 H), 4.48 (s, 2 H), 7.00 (d, J = 8.2 Hz, |
| | benzothiazol-3(2H)-yl)acetamide | | 1 H), 7.11 (d, J = 8.1 Hz, 1 H), 7.26 (s, 1 |
| | | | H), 7.41 (s, 1 H), 7.67 (s, 1 H) |
| 50 | 6-fluoro-3-(1H-imidazol-1- | 234 | 6.15 (s, 2 H), 6.98 (s, 1 H), 7.19 (m, 1 H), |
| | ylmethyl)-1,3-benzoxazol-2(3H)- | | 7.35 (s, 1 H), 7.49 (dd, J = 8.5, 2.4 Hz, 1 H), |
| | one | | 7.63 (dd, J = 8.7, 4.6 Hz, 1 H), 7.98 (s, 1 H) |
| 51 | 1-(1H-imidazol-4- | 215 | 5.11 (s, 1 H), 5.21 (s, 2 H), 6.52 (m, 1 H), |
| | ylmethyl)pyrazolo[1,5-a]pyridin- | | 7.02 (s, 1 H), 7.15 (m, 2 H), 7.56 (s, 1 H), |
| | 2(1H)-one | | 8.48 (d, J = 6.5 Hz, 1 H), 12.02 (m, 1 H). |
| | 6-chloro-2-(1H-imidazol-4- | 262/ | 3.59 (m, 2 H), 4.51 (m, 4 H), 6.95 (s, 1 H), |
| | ylmethyl)-1,4-dihydroisoquinolin- | 264 | 7.26 (s, 2 H), 7.32 (s, 1 H), 7.56 (s, 1 H), |
| | 3(2H)-one | | 11.92 (s, 1 H) |
| 52 | 2-(6-chloro-3-oxo-3,4- | 251/ | 1.29 (d, J = 7.28 Hz, 3 H), 3.59 (m, 2 H), |
| | dihydroisoquinolin-2(1H)- | 253 | 4.44 (s, 2 H), 5.03 (q, J = 7.28 Hz, 1 H), |
| | yl)propanamide | | 7.05 (s, 1 H), 7.31 (m, 4 H) |
| 53 | 5-chloro-2-(1H-imidazol-4- | 262/ | 3.60 (s, 2 H), 4.55 (s, 2 H), 4.57 (s, 2 H), |
| | ylmethyl)-1,4-dihydroisoquinolin- | 264 | 6.96 (s, 1 H), 7.24 (m, 2 H), 7.38 (d, J = 7.3 Hz, |
| | 3(2H)-one | | 1 H), 7.56 (s, 1 H), 11.9 (m, 1 H) |
| 54 | 2-(6-chloro-2-oxo-3,4- | (238/ | 2.58 (m, 2 H), 2.90 (m, 2 H), 4.40 (s, 2 H), |
| | dihydroquinolin-1(2H)-yl)acetamide | 240) | 6.82 (d, J = 8.5 Hz, 1 H), 7.14 (s (broad), 1 |
| | | | H), 7.24-7.32 (m, 2 H), 7.48 (s (broad), 1 H) |
| 55 | 2-(6-bromo-2-oxo-3,4- | (282/ | 2.54 (m, 2 H), 2.86 (m, 2 H), 4.36 (s, 2 H), |
| | dihydroquinolin-1(2H)-yl)acetamide | 284) | 6.74 (d, J = 8.5 Hz, 1 H), 7.11 (s (broad), 1 |
| | | | H), 7.39 (m, 2 H), 7.45 (s (broad), 1 H) |
| 56 | 1-(1H-imidazol-4-ylmethyl)-3,4- | 228 | 2.66 (m, 2 H), 2.95 (m, 2 H), 5.20 (s, 2 H), |
| | dihydroquinolin-2(1H)-one | | 7.05 (m, 2 H), 7.20 (m, 2 H), 7.52 (s, 1 H), |
| | hydrochloride | | 9.01 (s, 1 H) |
| 57 | 2-(6-iodo-2-oxo-3,4- | (330) | 2.53 (m, 2 H), 2.83 (m, 2 H), 4.34 (s, 2 H), |
| | dihydroquinolin-1(2H)-yl)acetamide | | 6.60 (d, J = 8.3 Hz, 1 H), 7.11 (s (broad), 1 |
| | | | H), 7.49 (s (broad), 1 H), 7.50 (m, 1 H), |
| | | | 7.52 (m, 1 H) |
| 58 | 2-(6-cyano-2-oxo-3,4- | (229) | 2.76 (m, 2 H), 3.08 (m, 2 H), 4.39 (s, 2 H), |
| | dihydroquinolin-1(2H)-yl)acetamide | | 7.10 (d, J = 9.2 Hz, 1 H), 7.32 (s (broad), 1 |
| | | | H), 7.68 (s (broad), 1 H), 7.84 (m, 2 H) |
| 59 | 7-chloro-2-{[2- | 394/ | (CDCl₃): 3.01 (m, 2 H), 3.15 (m, 2 H), |
| | (trifluoromethyl)imidazo[1,2- | 396 | 4.36 (s, 2 H), 5.15 (s, 2 H), 6.53 (d, J = 8.32 Hz, |
| | a]pyridin-3-yl]methyl}-1,2,4,5- | | 1 H), 6.83 (m, 2 H), 6.97 (m, 1 H), 7.27 (m, |
| | tetrahydro-3H-2-benzazepin-3-one | | 1 H), 7.58 (d, J = 9.29 Hz, 1 H), 8.19 (d, J = 6.85 Hz, |
| | | | 1 H) |
| 60 | 7-chloro-2-(1H-imidazol-4- | 276/ | 2.87 (m, 2 H), 3.08 (m, 2 H), 4.64 (d, 16.31 Hz, |
| | ylmethyl)-1,2,4,5-tetrahydro-3H-2- | 278 | 4 H), 7.15 (s, 2 H), 7.24 (s, 1 H), 7.46 (s, |
| | benzazepin-3-one hydrochloride | | 1 H), 8.97 (d, 1.00 Hz, 1 H), 14.48 (s, 1 H) |
| 61 | 7-chloro-3-(1H-imidazol-4- | 276/ | 3.02 (m, 2 H), 3.83 (m, 2 H), 3.92 (s, 2 H), |
| | ylmethyl)-1,3,4,5-tetrahydro-2H-3- | 278 | 4.60 (s, 2 H), 7.20 (m, 3 H), 7.55 (s, 1 H), |
| | benzazepin-2-one hydrochloride | | 9.02 (d, 1.25 Hz, 1 H), 14.57 (s, 1 H) |
| 62 | 7-chloro-3-{[2- | 394/ | 2.85 (m, 2 H), 3.61 (m, 2 H), 3.95 (s, 2 H), |
| | (trifluoromethyl)imidazo[1,2- | 396 | 5.08 (s, 2 H), 7.06 (m, 2 H), 7.18 (m, 2 H), |
| | a]pyridin-3-yl]methyl}-1,3,4,5- | | 7.46 (m, 1 H), 7.72 (d, J = 9.03 Hz, 1 H), |
| | tetrahydro-2H-3-benzazepin-2-one | | 8.39 (d, J = 6.78 Hz, 1 H) |
| 63 | 1-[(5-fluoro-2-phenyl-1H-indol-1- | 323 | 1.53 (m, 4 H), 2.32 (t, J = 6.5 Hz, 2 H), |
| | yl)methyl]piperidin-2-one | | 2.49 (t, J = 6.0 Hz, 2 H), 5.90 (s, 2 H), 6.50 (s, 1 |
| | | | H), 6.97 (td, J = 9.3, 2.5 Hz, 1 H), 7.25 (m, 1 |
| | | | H), 7.49 (m, 6 H) |
| 64 | 1-[(2-phenyl-1H-indol-1- | 305 | 1.53 (m, 4 H), 2.32 (t, J = 6.5 Hz, 2 H), |
| | yl)methyl]piperidin-2-one | | 2.51 (t, J = 6.0 Hz, 2 H), 5.94 (s, 2 H), 6.55 (s, 1 |
| | | | H), 7.20 (m, 2 H), 7.46 (m, 5 H), 7.61 (dd, J = 16.3, |
| | | | 7.8 Hz, 2 H) |
| 65 | [(2E)-1-(1H-imidazol-4-ylmethyl)-4- | 232 | 0.85 (t, J = 7.3 Hz, 3 H), 1.22 (m, 2 H), |
| | propylpyrrolidin-2- | | 1.34 (m, 2 H), 2.36 (m, 1 H), 2.48 (m, 1 H), |
| | ylidene]cyanamide | | 2.99 (dd, J = 16.8, 8.5 Hz, 1 H), 3.19 (dd, J = 9.8, |
| | | | 7.0 Hz, 1 H), 3.64 (t, J = 9.3 Hz, 1 H), |
| | | | 4.37 (s, 2 H), 7.06 (s, 1 H), 7.60 (s, 1 H), |
| | | | 12.02 (m, 1 H) |
| 66 | 2-[(2E)-2-(cyanoimino)-4- | 209 | (CDCl₃): 0.94 (t, J = 7.3 Hz, 3 H), 1.34 (m, 2 |
| | propylpyrrolidin-1-yl]acetamide | | H), 1.49 (m, 2 H), 2.57 (m, 2 H), 3.13 (m, 1 |
| | | | H), 3.39 (dd, J = 10.3, 7.0 Hz, 1 H), |
| | | | 3.79 (dd, J = 10.0, 8.3 Hz, 1 H), 4.06 (s, 2 H), |
| | | | 5.72 (s, 1 H), 6.41 (s, 1 H) |
| 67 | 2-[(2E)-2-(cyanoimino)-4- | 223 | (CDCl₃): 0.93 (td, J = 7.3, 3.5 Hz, 3 H), |
| | propylpyrrolidin-1-yl]propanamide | | 1.34 (m, 2 H), 1.44 (m, 5 H), 2.52 (m, 2 H), |
| | | | 3.13 (m, 1 H), 3.28 (ddd, J = 21.6, 10.3, 6.8 Hz, 1 |
| | | | H), 3.76 (ddd, J = 14.1, 10.3, 8.0 Hz, 1 H), |
| | | | 5.56 (s, 1 H), 4.80 (m, 1 H), 6.41 (m, 1 H) |
| 68 | 2-[(2E)-2-(cyanoimino)-4- | 237 | 0.94 (m, 6 H), 1.34 (m, 2 H), 1.45 (m, 2 H), |
| | propylpyrrolidin-1-yl]butanamide | | 1.72 (m, 1 H), 1.99 (m, 1 H), 2.51 (m, 2 H), |
| | | | 3.22 (m, 2 H), 3.75 (ddd, J = 54.2, 10.5, 8.0 Hz, |
| | | | 1 H), 4.56 (m, 1 H), 5.55 (s, 1 H), |
| | | | 6.34 (m, 1 H) |
| 69 | N-[(2E)-1-(1H-imidazol-4-ylmethyl)- | 285 | (CD₃OD): 0.91 (t, J = 7.3 Hz, 3 H), 1.32 (m, |
| | 4-propylpyrrolidin-2- | | 2 H), 1.40 (m, 2 H), 2.40 (m, 1 H), 2.65 (dd, |
| | ylidene]methanesulfonamide | | J = 17.6, 7.5 Hz, 1 H), 2.99 (s, 3 H), 3.11 |
| | | | (dd, J = 10.5, 7.0 Hz, 1 H), 3.24 (dd, J = 17.8, |
| | | | 8.5 Hz, 1 H), 3.58 (dd, J = 10.5, 8.0 Hz, |
| | | | 1 H), 4.54 (s, 2 H), 7.11 (s, 1 H), |
| | | | 7.68 (d, J = 0.5 Hz, 1 H) |

### Example 36

### LBS Binding Assay

[LBS stands for Levetiracetam Binding Site cf. M. Noyer et al., Eur. J. Pharmacol. (1995), 286, 137-146.]

The inhibition constant (Kᵢ) of a compound is determined in competitive binding experiments by measuring the binding of a single concentration of a radioactive ligand at equilibrium with various concentrations of the unlabeled test substance. The concentration of the test substance inhibiting 50% of the specific binding of the radioligand is called the IC₅₀. The equilibrium dissociation constant Kᵢ is proportional to the IC₅₀ and is calculated using the equation of Cheng and Prusoff (Cheng Y. et al., Biochem. Pharmacol. (1972), 22, 3099-3108).

The concentration range usually encompasses 6 log units with variable steps (0.3 to 0.5 log). Assays are performed in mono- or duplicate, each Kᵢ determination is performed on two different samples of test substance.

Cerebral cortex from 200-250 g male Sprague-Dawley rats are homogenised using a Potter S homogeniser (10 strokes at 1,000 rpm; Braun, Germany) in 20 mmol/l Tris-HCl (pH 7.4), 250 mmol/l sucrose (buffer A); all operations are performed at 4° C. The homogenate is centrifuged at 30,000 g for 15 min. The crude membrane pellet obtained is resuspended in 50 mmol/l Tris-HCl (pH 7.4), (buffer B) and incubated 15 min at 37° C., centrifuged at 30,000 g for 15 min and washed twice with the same buffer. The final pellet is resuspended in buffer A at a protein concentration ranging from 15 to 25 mg/ml and stored in liquid nitrogen.

Membranes (150-200 μg of protein/assay) are incubated at 4° C. for 120 min in 0.5 ml of a 50 mmol/l Tris-HCl buffer (pH 7.4) containing 2 mmol/l MgCl₂, 1 to 2 10⁻⁹ mol/l of [³H]-2-[4-(3-azidophenyl)-2-oxo-1-pyrrolidinyl]butanamide and increasing concentrations of the test compound of formula (I). The non specific binding (NSB) is defined as the residual binding observed in the presence of a concentration of reference substance (e.g. 10⁻³ mol/l levetiracetam) that binds essentially all the receptors. Membrane-bound and free radioligands are separated by rapid filtration through glass fiber filters (equivalent to Whatman GF/C or GF/B; VEL, Belgium) pre-soaked in 0.1% polyethyleneimine and 10⁻³ mol/l levetiracetam to reduce non specific binding. Samples and filters are rinsed by at least 6 ml of 50 mmol/l Tris-HCl (pH 7.4) buffer. The entire filtration procedure does not exceed 10 seconds per sample. The radioactivity trapped onto the filters is counted by liquid scintillation in a 13-counter (Tri-Carb 1900 or TopCount 9206, Camberra Packard, Belgium, or any other equivalent counter). Data analysis is performed by a computerized non linear curve fitting method using a set of equations describing several binding models assuming populations of independent non-interacting receptors, which obey the law of mass.

### Example 37

### Animal Model of Sound-Susceptible Mice

The objective of this test is to evaluate the anticonvulsant potency of a compound in sound-susceptible mice, a genetic animal model with reflex seizures. In this model of primary generalised epilepsy, seizures are evoked without electrical or chemical stimulation and the seizure types are, at least in part, similar in their clinical phenomenology to seizures occurring in man (Löscher W. & Schmidt D., Epilepsy Res. (1998), 2, 145-181; Buchhalter J. R., Epilepsia (1993), 34, S31-S41).

Male or female genetically sound-sensitive mice (14-28 g; N=10), derived from a DBA strain originally selected by Dr. Lehmann of the Laboratory of Acoustic Physiology (Paris) and bred in the UCB Pharma Sector husbandry unit since 1978, are used. The experimental design consisted of several groups, one group receiving the vehicle control and the other groups different doses of the test-compound. The compounds are administered intraperitoneally 60 minutes before the induction of audiogenic seizures. The range of the doses administered had a logarithmic progression, generally between 1.0×10⁻⁵ mol/kg and 1.0×10⁻³ mol/kg, but lower or higher doses are tested if necessary.

For testing, the animals are placed in small cages, one mouse per cage, in a sound-attenuated chamber. After a period of orientation of 30 seconds, the acoustic stimulus (90 dB, 10-20 kHz) is delivered for 30 seconds via loudspeakers positioned above each cage. During this interval, the mice are observed and the presence of the 3 phases of the seizure activity namely wild running, clonic and tonic convulsions, is recorded. The proportion of mice protected against wild running, clonic and tonic convulsions, respectively, is calculated.

For active compounds, an ED₅₀ value, i.e. the dose producing 50% protection relative to the control group, together with 95% confidence limits, is calculated using a Probit Analysis (SAS/STAT® Software, version 6.09, PROBIT procedure) of the proportions of protected mice for each of the 3 phases of the seizure activity.

Compounds synthesized according to the procedure described in examples 1 to 33 and described in table 1 are tested in the SV2 binding assay and/or audiogenic seizure in mice, according to the procedure described above, and are found active.

1. A method of treating or preventing a disease in a human, the method comprising administering to the human an effective amount of a compound having the formula (I), 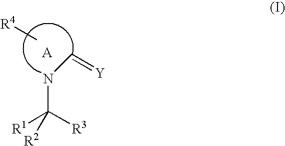
or a geometrical isomer, an enantiomer, a diastereomer or a mixture of diastereomers, or a pharmaceutically acceptable salt of one of the foregoing, wherein
Y is O, S or NR⁸;
R¹ is hydrogen or C₁₋₆ alkyl;
R² is hydrogen;
R³ is —CONR⁵R⁶, —COR⁷, an imidazolyl, an imidazopyridinyl, an imidazopyridazinyl or a 1H-indol-1-yl;
R⁵, R⁶ are the same or different and are independently selected from hydrogen and C₁₋₆ alkyl;
R⁷ is a C₁₋₆ alkyl;
R⁸ is CN or C₁₋₆ alkylsulfonyl;
A is imidazolidin-1-yl, 1,3-oxazolidin-3-yl, 2,5-dihydro-1H-pyrrol-1-yl, 1,3-thiazol-3(2H)-yl, 1,3-thiazolidin-3-yl, pyrrolidin-1-yl, piperidin-1-yl, azepan-1-yl, 5,6-dihydro-4H-thieno[3,2-b]pyrrol-4-yl, hexahydro-4H-thieno[3,2-b]pyrrol-4-yl, 2,3-dihydro-1H-thieno[3,4-b]pyrrol-1-yl, 1,3-benzothiazol-3(2H)-yl, 1,3-benzoxazol-3(2H)-yl, pyrazolo[1,5-a]pyridin-1(2H)-yl, 3,4-dihydroisoquinolin-2(1H)-yl, 3,4-dihydroquinolin-1(2H)-yl, 1,3,4,5-tetrahydro-2H-2-benzazepin-2-yl, or 1,2,4,5-tetrahydro-3H-3-benzazepin-3-yl;
R⁴ is either R^{4a} or R^{4b} depending on whether A being is a monocyclic or a bicyclic heterocycle:
when A is a monocyclic heterocyclic moiety, R⁴ is R⁴ which is hydrogen; C₁₋₆ alkyl optionally substituted by halogen, C₁₋₄ alkoxy, C₁₋₄ alkylthio, azido, nitrooxy or aryl; C₂₋₆ alkenyl optionally substituted by halogen; C₂₋₆ alkynyl optionally substituted by halogen; azido; alkoxycarbonylamino; arylsulfonyloxy; a substituted or unsubstituted aryl; or a 3-8 membered substituted or unsubstituted heterocycle;
when A is a bicyclic heterocyclic moiety, R⁴ is R^{4b}, which is hydrogen; nitro; cyano; halogen; heterocycle; amino; aryl; C₁-₆ alkyl optionally substituted by at least one halogen; or C₁₋₆ alkoxy optionally substituted by at least one halogen;
with the proviso that
when A=Y is 2-oxo-piperidin-1-yl, a 2-oxo-azepan-1-yl, a 2-oxo-1,3-benzothiazol-3(2H)-yl or a 2-oxo-1,3-benzoxazol-3(2H)-yl, R³ imidazolyl, imidazopyridinyl, imidazopyridazinyl or 1H-indol-1-yl;
when A=Y is 5-oxoimidazolidin-1-yl, R¹ and R² are hydrogen, and R³ is —CONR⁵R⁶, R⁵ and R⁶ are as above defined then R^{4a} is not alkyl, aralkyl or substituted aralkyl;
when A=Y is 2 oxo-piperidin-1-yl or 2-oxo-azepan-1-yl, and R¹, R² and R^{4a} are hydrogen, then R³ is not 2-phenylimidazo[1,2-a]pyridin-3-yl;
when A is pyrrolidin-1-yl, Y is NR⁸.
when A is pyrrolidin-1-yl, piperidin-1-yl or azepan-1-yl, R³ is —CONR⁵R⁶ or —COR⁷, Y is NR⁸ and R⁸ is CN, then R^{4a} is not hydrogen.
wherein the disease is epilepsy, epileptogenesis, seizure disorders, convulsions, Parkinson's disease, dyskinesia induced by dopamine replacement therapy, tardive dyskinesia induced by administration of neuroleptic drugs, Huntington Chorea, and other neurological disorders including bipolar disorders, mania, depression, anxiety, panic disorders, attention deficit hyperactivity disorder (ADHD), migraine, trigeminal and other neuralgia, chronic pain, neuropathic pain, cerebral ischemia, cardiac arrhythmia, myotonia, cocaine abuse, stroke, myoclonus, tremor, essential tremor, simple or complex tics, Tourette syndrome, restless leg syndrome and other movement disorders, neonatal cerebral haemorrhage, amyotrophic lateral sclerosis, spasticity and degenerative diseases, subjective tinnitus, apathy syndrome, bronchial asthma, asthmatic status and allergic bronchitis, asthmatic syndrome, bronchial hyperreactivity and bronchospastic syndromes, lower urinary tract disorders, as well as allergic and vasomotor rhinitis, or rhinoconjunctivitis.

2. The method according to claim 1 wherein
Y is O or S;
R¹ is hydrogen or C₁₋₆ alkyl;
R² is hydrogen;
R³ is —CONR⁵R⁶, —COR⁷, an imidazolyl, an imidazopyridinyl, an imidazopyridazinyl;
R⁵, R⁶ are independently hydrogen or C₁₋₆ alkyl;
R⁷ is a C₁₋₆ alkyl;
A is imidazolidin-1-yl, 1,3-oxazolidin-3-yl, 2,5-dihydro-1H-pyrrol-1-yl, 1,3-thiazol-3(2H)-yl, 2-oxo-1,3-thiazolidin-3-yl, piperidin-1-yl, azepan-1-yl, 5,6-dihydro-4H-thieno[3,2-b]pyrrol-4-yl, hexahydro-4H-thieno[3,2-b]pyrrol-4-yl, 2,3-dihydro-1H-thieno[3,4-b]pyrrol-1-yl, 1,3-benzothiazol-3(2H)-yl, 1,3-benzoxazol-3(2H)-yl, pyrazolo[1,5-a]pyridin-1(2H)-yl, 3,4-dihydroisoquinolin-2(1H)-yl, 3,4-dihydroquinolin-1(2H)-yl, 1,3,4,5-tetrahydro-2H-2-benzazepin-2-yl, 1,2,4,5-tetrahydro-3H-3-benzazepin-3-yl;
R⁴ is either R^{4a} or R^{4b} depending on whether A being is a monocyclic or a bicyclic heterocycle:
when A is a monocyclic heterocyclic moiety, R⁴ is R⁴ which is hydrogen; C₁₋₆ alkyl optionally substituted by halogen, C₁₋₄ alkoxy, C₁₋₄ alkylthio, azido, nitrooxy or aryl; C₂₋₆ alkenyl optionally substituted by halogen; C₂₋₆ alkynyl optionally substituted by halogen; azido; alkoxycarbonylamino; arylsulfonyloxy; a-substituted or unsubstituted aryl; or a 3-8 membered substituted or unsubstituted heterocycle;
when A is a bicyclic heterocyclic moiety, R⁴ is R^{4b}, which is hydrogen; nitro; cyano; halogen; heterocycle; amino; aryl; C₁-₆ alkyl optionally substituted by at least one halogen; or C₁₋₆ alkoxy optionally substituted by at least one halogen;
with the proviso that
when A=Y is 2-oxo-piperidin-1-yl, 2-oxo-azepan-1-yl, 2-oxo-1,3-benzothiazol-3(2H)-yl or 2-oxo-1,3-benzoxazol-3(2H)-yl, R³ is imidazolyl, imidazopyridinyl or imidazopyridazinyl;
when A=Y is 5-oxoimidazolidin-1-yl, R¹ and R² are hydrogen, and R³ is —CONR⁵R⁶, R⁵ and R⁶ are as above defined then R^{4a} is not alkyl, aralkyl or substituted aralkyl;
when A=Y is 2 oxo-piperidin-1-yl or 2-oxo-azepan-1-yl, and R¹, R² and R^{4a} are hydrogen, then R³ is not 2-phenylimidazo[1,2-a]pyridin-3-yl.

3. The method according to claim 1, wherein A=Y is 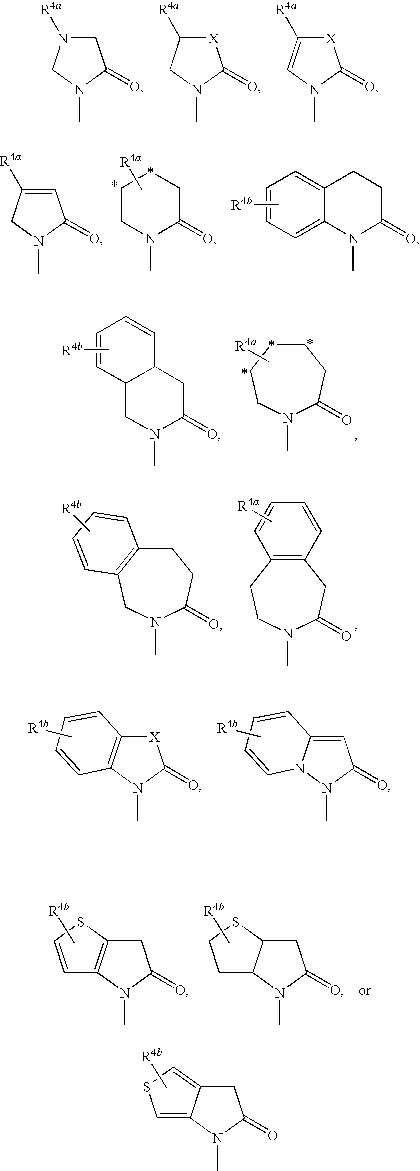
and wherein X is O or S, R^{4a}/R^{4b} are as above defined and the asterisks indicate the attachment sites of R^{4a}.

4. The method according to claim 1, wherein A=Y is 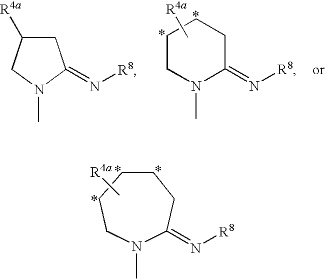
wherein R⁸ is cyano or alkylsulfonyl and R^{4a}/R^{4b} are as above defined and the asterisks indicate the attachment sites of R^{4a}.

5. The method according to claim 1 wherein the disease is epilepsy.

6. The method according to claim 1, wherein Y is O.

7. The method according to claim 1, wherein R¹ is hydrogen, methyl or ethyl and R² is hydrogen.

8. The method according to claim 1, wherein R³ is —CONH₂.

9. The method according to claim 1 wherein R³ is 1H-imidazol-1-yl, 1H-imidazol-4-yl, 1H-imidazol-5-yl, imidazo[1,2-a]pyridin-3-yl, an 1H-indol-1-yl or imidazo[1,2-b]pyridazin-3-yl.

10. The method according claim 1, wherein R^{4a} is hydrogen; C₁₋₆ alkyl optionally substituted by halogen; or a phenyl.

11. The method according to claim 1 wherein R^{4b} is hydrogen, halogen, nitro, cyano, or C₁₋₆ alkyl optionally substituted by halogen.

12. A compound having the formula (I-A), 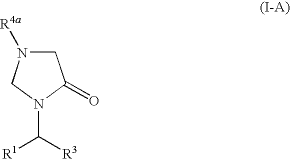
or a geometrical isomer, an enantiomer, a diastereomer or a mixture of diastereomers, or a pharmaceutically acceptable salt of one of the foregoing, wherein
R¹ is hydrogen or C₁₋₆ alkyl;
R³ is —CONH₂, imidazolyl, imidazopyridinyl, imidazopyridazinyl;
R^{4a} is either hydrogen or aryl;
with the proviso that 2-(5-oxoimidazolidin-1-yl)acetamide is excluded.

13. A compound having the formula (I-B 1 or I-B2), 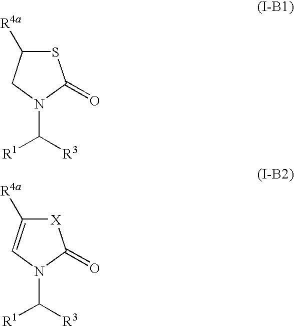
or a geometrical isomer, an enantiomer, a diastereomer or a mixture of diastereomers, or a pharmaceutically acceptable salt of one of the foregoing, wherein
X is either S or O;
R¹ is hydrogen or C₁₋₆ alkyl;
R³ is —CONH₂, imidazolyl, imidazopyridinyl, imidazopyridazinyl;
R^{4a} is hydrogen; C₁₋₆ alkyl optionally substituted by halogen or C₁₋₄ alkoxy; aryl; or C₂₋₆ alkenyl optionally substituted by halogen.

14. A compound having the formula (I-B3), 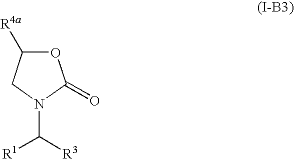
or a geometrical isomer, an enantiomer, a diastereomer or a mixture of diastereomers, or a pharmaceutically acceptable salt of one of the foregoing, wherein
R¹ is hydrogen or C₁₋₆ alkyl;
R³ is —CONH₂, imidazolyl, imidazopyridinyl, imidazopyridazinyl;
R^{4a} is C₁₋₆ alkyl optionally substituted by halogen or C₁₋₄ alkoxy; aryl; or C₂₋₆ alkenyl optionally substituted by halogen.

15. A compound having the formula (I-C), 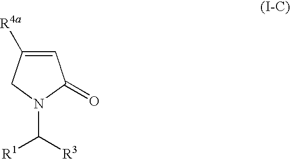
or a geometrical isomer, an enantiomer, a diastereomer or a mixture of diastereomers, or a pharmaceutically acceptable salt of one of the foregoing, wherein
R¹ is hydrogen or C₁₋₆ alkyl;
R³ is —CONH₂, imidazolyl, imidazopyridinyl, imidazopyridazinyl;
R^{4a} is methyl, ethyl, butyl optionally substituted by halogen or C₁₋₄ alkoxy, an unsubstituted phenyl or a phenyl substituted by halogen, a C₁₋₆ alkyl optionally substituted by halogen or a C₁₋₄ alkoxy; or R^{4a} is a C₂₋₆ alkenyl optionally substituted by halogen.

16. A compound having the formula (I-D1 or I-D2), 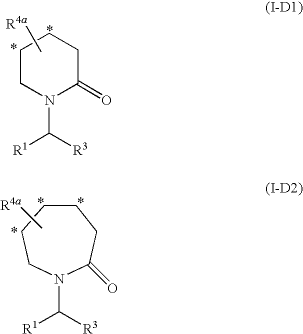
or a geometrical isomer, an enantiomer, a diastereomer or a mixture of diastereomers, or a pharmaceutically acceptable salt of one of the foregoing, wherein
R¹ is hydrogen or C₁₋₆ alkyl;
R³ is imidazolyl, imidazopyridinyl, imidazopyridazinyl or 1H-indol-1-yl;
R^{4a} is hydrogen, C₁₋₆ alkyl optionally substituted by halogen or C₁₋₄ alkoxy; aryl; or C₂₋₆ alkenyl optionally substituted by halogen,
with the proviso that when R¹ and R^{4a} are hydrogen, R³ is not 2-phenylimidazo[1,2-a]pyridin-3-yl.

17. A compound having the formula (I-F1, I-F2 or I-F3), 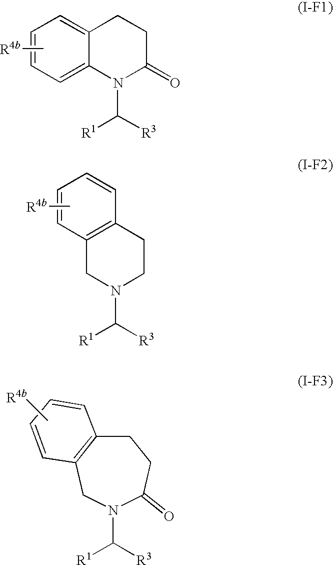
or a geometrical isomer, an enantiomer, a diastereomer or a mixture of diastereomers, or a pharmaceutically acceptable salt of one of the foregoing, wherein
R¹ is hydrogen or C₁₋₆ alkyl;
R³ is —CONH₂, imidazolyl, imidazopyridinyl or imidazopyridazinyl;
R^{4b} is hydrogen; halogen; nitro; cyano; C₁₋₄ alkyl optionally substituted by halogen; C₁₋₄ alkoxy optionally substituted by halogen.

18. A compound having the formula (I-F4), 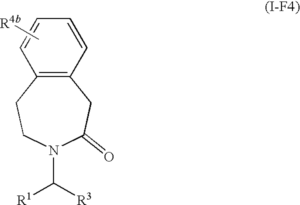
or a geometrical isomer, an enantiomer, a diastereomer or a mixture of diastereomers, or a pharmaceutically acceptable salt of one of the foregoing, wherein
R¹ is hydrogen or C₁₋₆ alkyl;
R³ is imidazolyl, imidazopyridinyl or imidazopyridazinyl;
R^{4b} is hydrogen; halogen; nitro; cyano; C₁₋₄ alkyl optionally substituted by halogen; C₁₋₄ alkoxy optionally substituted by halogen;

19. A compound having either of the formula (I-G1, I-G2 or I-G3), 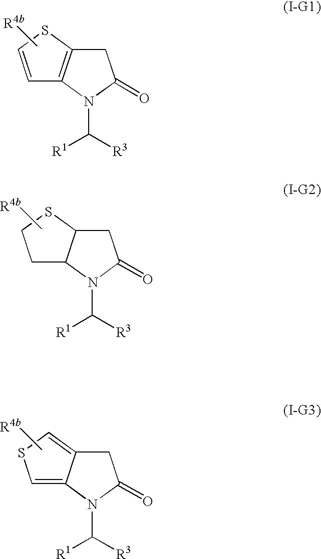
or a geometrical isomer, an enantiomer, a diastereomer or a mixture of diastereomers, or a pharmaceutically acceptable salt of one of the foregoing, wherein
R¹ is hydrogen or C₁₋₆ alkyl;
R³ is —CONH₂, imidazolyl, imidazopyridinyl, imidazopyridazinyl;
R^{4b} is hydrogen; halogen; C₁₋₄ alkyl optionally substituted by halogen; C₁₋₄ alkoxy optionally substituted by halogen.

20. A compound having either of the formulae (I-H1, I-H2 or I-H3), 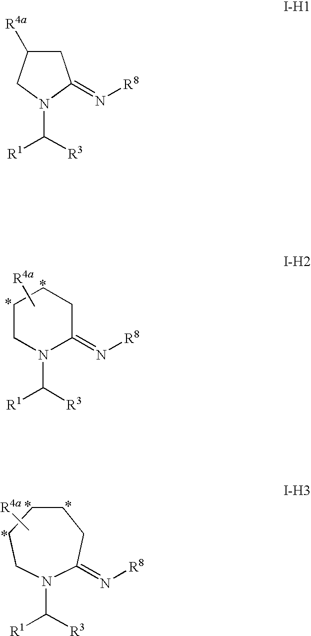
or a geometrical isomer, an enantiomer, a diastereomer or a mixture of diastereomers, or a pharmaceutically acceptable salt of one of the foregoing, wherein
R¹ is hydrogen or C₁₋₆ alkyl;
R³ is —CONH₂ or imidazolyl;
R⁸ is cyano or C₁₋₆ alkylsulfonyl;
R^{4a} is hydrogen, C₁₋₆ alkyl optionally substituted by halogen or C₁₋₄ alkoxy; aryl; or C₂₋₆ alkenyl optionally substituted by halogen;
with the proviso that when R⁸ is CN and R³ is —CONH₂, then R^{4a} is not hydrogen.

21. The compound according to claim 12 which is (2S)-2-{[(benzylamino)acetyl]amino}butanamide; (2S)-2-(3-benzyl-5-oxoimidazolidin-1-yl)butanamide; (2S)-2-(5-oxoimidazolidin-1-yl)butanamide; (2S)-2-[3-(4-aminophenyl)-5-oxoimidazolidin-1-yl]butanamide; (2S)-2-(allylamino)butanamide; methyl allyl[(1S)-1-(aminocarbonyl)propyl]carbamate; (2S)-2-(1H-pyrrol-1-yl)butanamide; methyl 4-{[(1S)-1-(aminocarbonyl)propyl]amino}-3-phenylbutanoate; S-(1-formylbutyl)O-methyl thiocarbonate; 2-(2,4-dioxo-5-propyl-1,3-thiazolidin-3-yl)propanamide; 2-(4-hydroxy-2-oxo-5-propyl-1,3-thiazolidin-3-yl)propanamide; 1-cyanopentyl 4-methylbenzenesulfonate; S-[cyano(phenyl)methyl]ethanethioate; S-(1-cyanopentyl)ethanethioate; 5-butyl-1,3-thiazolidin-2-one; 5-propyl-1,3-thiazolidin-2-one; (2S)-2-[2-thioxo-5-(2,2,2-trifluoroethyl)-1,3-thiazolidin-3-yl]butanamide; 1-(hydroxymethyl)-5-phenylpiperidin-2-one; 6-fluoro-3-(hydroxymethyl)-1,3-benzoxazol-2(3H)-one; 1-(hydroxymethyl)-5-propylpiperidin-2-one; 1-(hydroxymethyl)-4-phenylpiperidin-2-one; 1-(hydroxymethyl)-4-propylpiperidin-2-one; 1-(chloromethyl)-5-phenylpiperidin-2-one; 3-(chloromethyl)-6-fluoro-1,3-benzoxazol-2(3H)-one; 1-(chloromethyl)-5-propylpiperidin-2-one; 1-(chloromethyl)-4-phenylpiperidin-2-one; ethyl 4-formylheptanoate; ethyl 4-{[(tert-butoxycarbonyl)amino]methyl}heptanoate; ethyl 4-(aminomethyl)heptanoate hydrochloride; ethyl 3-(2-bromoethyl)hexanoate; tert-butyl 2-oxo-5-propylazepane-1-carboxylate; tert-butyl 2-oxo-5-phenylazepane-1-carboxylate; 4-propylazepan-2-one; 6-propylazepan-2-one; tert-butyl 2-oxo-4-propylazepane-1-carboxylate; tert-butyl 2-oxo-6-propylazepane-1-carboxylate; methyl 4-{2-[(tert-butoxycarbonyl)amino]ethyl}heptanoate; methyl 6-[(tert-butoxycarbonyl)amino]-4-phenylhexanoate; methyl 5-{[(tert-butoxycarbonyl)amino]methyl}octanoate; methyl 6-[(tert-butoxycarbonyl)amino]-3-propylhexanoate; methyl 4-(2-aminoethyl)heptanoate hydrochloride; methyl 6-amino-4-phenylhexanoate; methyl 5-(aminomethyl)octanoate hydrochloride; methyl 6-amino-3-propylhexanoate hydrochloride; methyl 4-{2-[(1H-imidazol-4-ylmethyl)amino]ethyl}heptanoate; isopropyl 4-[2-({[2-(trifluoromethyl)imidazo[1,2-a]pyridin-3-yl]methyl}amino)ethyl]heptanoate; isopropyl 4-phenyl-6-({[2-(trifluoromethyl)imidazo[1,2-a]pyridin-3-yl]methyl}amino)hexanoate; methyl 5-{[(1H-imidazol-4-ylmethyl)amino]methyl}octanoate; methyl 6-[(1H-imidazol-4-ylmethyl)amino]-3-propylhexanoate; 4-{2-[(1H-imidazol-4-ylmethyl)amino]ethyl}heptanoic acid dihydrochloride; 4-[2-({[2-(trifluoromethyl)imidazo[1,2-a]pyridin-3-yl]methyl}amino)ethyl]heptanoic acid dihydrochloride; 4-phenyl-6-({[2-(trifluoromethyl)imidazo[1,2-a]pyridin-3-yl]methyl}amino)hexanoic acid dihydrochloride; 5-{[(1H-imidazol-4-ylmethyl)amino]methyl}octanoic acid dihydrochloride; 6-[(1H-imidazol-4-ylmethyl)amino]-3-propylhexanoic acid dihydrochloride; di-tert-butyl(3-nitro-2-thienyl)malonate; di-tert-butyl(3-amino-2-thienyl)malonate; di-tert-butyl{3-[(1H-imidazol-4-ylmethyl)amino]-2-thienyl}malonate; {3-[(1H-imidazol-4-ylmethyl)amino]-2-thienyl}acetic acid dihydrochloride; [3-({[2-(trifluoromethyl)imidazo[1,2-a]pyridin-3-yl]methylene}amino)-2-thienyl]acetic acid; [3-({[2-(trifluoromethyl)imidazo[1,2-a]pyridin-3-yl]methyl}amino)-2-thienyl]acetic acid; ethyl{4-[(tert-butoxycarbonyl)amino]-3-thienyl}acetate; ethyl(4-amino-3-thienyl)acetate hydrochloride; ethyl{4-[(1H-imidazol-4-ylmethyl)amino]-3-thienyl}acetate; sodium {4-[(1H-imidazol-4-ylmethyl)amino]-3-thienyl}acetate; 1-{(3-ethoxy-3-oxopropanoyl)[(1-trityl-1H-imidazol-4-yl)methyl]amino}pyridinium chloride; ethyl 2-oxo-1-[(1-trityl-1H-imidazol-4-yl)methyl]-1,2-dihydropyrazolo[1,5-a]pyridine-3-carboxylate-methanol (1:2); tert-butyl(5-chloro-2-methylphenyl)acetate; methyl(5-chloro-2-methylphenyl)acetate; methyl[2-(bromomethyl)-5-chlorophenyl]acetate; methyl 2-(6-chloro-3-oxo-3,4-dihydroisoquinolin-2(1H)-yl)propanoate; tert-butyl(2-chloro-6-methylphenyl)acetate; methyl(2-chloro-6-methylphenyl)acetate; methyl[2-(bromomethyl)-6-chlorophenyl]acetate; 1-[(1-trityl-1H-imidazol-4-yl)methyl]-3,4-dihydroquinolin-2(1H)-one; tert-butyl 7-chloro-3-oxo-1,3,4,5-tetrahydro-2H-2-benzazepine-2-carboxylate; tert-butyl 7-chloro-2-oxo-1,2,4,5-tetrahydro-3H-3-benzazepine-3-carboxylate; 3-(2-{[(tert-butoxycarbonyl)amino]methyl}-5-chlorophenyl)propanoic acid; (2-{2-[(tert-butoxycarbonyl)amino]ethyl}-4-chlorophenyl)acetic acid; 3-[2-(aminomethyl)-5-chlorophenyl]propanoic acid hydrochloride; [2-(2-aminoethyl)-4-chlorophenyl]acetic acid hydrochloride; 3-[5-chloro-2-({[(1-trityl-1H-imidazol-4-yl)methyl]amino}methyl)phenyl]propanoic acid, 1-[(5-fluoro-2-phenyl-1H-indol-1-yl)methyl]piperidin-2-one; 1-[(2-phenyl-1H-indol-1-yl)methyl]piperidin-2-one; [(2E)-1-(1H-imidazol-4-ylmethyl)-4-propylpyrrolidin-2-ylidene]cyanamide; 2-[(2E)-2-(cyanoimino)-4-propylpyrrolidin-1-yl]acetamide; 2-[(2E)-2-(cyanoimino)-4-propylpyrrolidin-1-yl]propanamide; 2-[(2E)-2-(cyanoimino)-4-propylpyrrolidin-1-yl]butanamide; or N-[(2E)-1-(1H-imidazol-4-ylmethyl)-4-propylpyrrolidin-2-ylidene]methanesulfonamide.

22. A pharmaceutical composition comprising a compound according to claim 12 or a pharmaceutically acceptable salt thereof in combination with a pharmaceutically acceptable diluent or carrier.
